(19) United States
(12) Patent Application Publication
(10) Pub. No.: US 2026/0327563 A1
(43) Pub. Date: Oct. 22, 2026

(54) SELECTION OF OPTIMAL CELL DONORS AND METHODS AND COMPOSITIONS FOR ENHANCED EXPANSION AND CYTOTOXICITY OF DONOR CELLS

(71) Applicant: NKARTA, INC., South San Francisco, CA (US)

(72) Inventors: Michael Whang, South San Francisco, CA (US); Ming-Hong Xie, South San Francisco, CA (US)

(21) Appl. No.: 19/106,877

(22) PCT Filed: Oct. 19, 2023
(86) PCT No.: PCT/US2023/077336

§ 371 Date: Feb. 26, 2025

Related U.S. Application Data
(60) Provisional application No. 63/380,532, filed on Oct. 21, 2022.

(51) Int. Cl. C12N 5/0783; C07K 16/28; G01N 33/569

(52) U.S. Cl. CPC C12N 5/0646; C07K 16/2803; G01N 33/56972; C12N 2501/2302; C12N 2501/2312; C12N 2501/2315; C12N 2501/2318; C12N 2510/00

(57) ABSTRACT

Several embodiments disclosed herein relate to methods and compositions for enhanced expansion of NK cells in culture, through the repeated co-culturing of NK cells with feeder cells and the selective use of stimulatory interleukins. In several embodiments, the methods utilize one or more soluble interleukins as culture media supplements at one or more time points during expansion of the NK cell, or other immune cell, which results in a highly expanded and highly cytotoxic population of cells, for use in, for example allogeneic cellular immunotherapy. Some embodiments also relate to methods of enriching for, or selecting donors having, NK cells that exhibit enhanced expansion in culture and/or enhanced cytotoxicity against target cells.

### CROSS-REFERENCE TO RELATED APPLICATIONS

This application claims priority to U.S. Provisional Patent Application No. 63/380,532, filed Oct. 21, 2022, the entire contents of which is incorporated by reference herein.

### FIELD

Some embodiments of the methods and compositions disclosed herein relate to identification of donors of Natural Killer (NK) cells that exhibit enhanced capacity for expansion in culture and/or enhanced cytotoxicity against target tumor cells, including after being engineered to express, for example anti-tumor marker directed chimeric antigen receptors. Some embodiments also relate to methods of enriching for, or selecting donors having, NK cells that exhibit enhanced expansion in culture and/or enhanced cytotoxicity against target cells.

### BACKGROUND

The use of engineered cells for cellular immunotherapy allows for treatment of cancers or other diseases by leveraging various aspects of the immune system to target and destroy diseased or damaged cells. Such therapies require engineered cells in numbers sufficient for therapeutically relevant doses.

### INCORPORATION BY REFERENCE OF MATERIAL IN SEQUENCE LISTING FILE

This application incorporates by reference the Sequence Listing contained in the following XML file being submitted concurrently herewith: File name: NKT094WO_ST26.xml; created Oct. 17, 2023 and is 177,873 bytes in size.

### SUMMARY

In several embodiments, there are provided various methods for enhancing the expansion of immune cells (e.g., NK cells) for use in cellular immunotherapy. For example, in several embodiments, there is provided a method in which immune cells expressing an inhibitory Killer Cell Ig Like Receptor KIR (iKIR) are enriched, e.g. prior to expansion. In several embodiments, biological samples containing threshold levels of immune cells expressing an iKIR are selected for subsequent expansion. In several embodiments, immune cells are co-cultured with a feeder cell line in a media supplemented with one or more soluble cytokines, the cytokines being added to the media at least once during the co-culture. In several embodiments, the immune cells are NK cells. In several embodiments, the expanded NK cells exhibit unexpectedly enhanced expansion. Further, in several embodiments, the expanded NK cells exhibit an unexpectedly enhanced natural cytotoxicity, independent of whether the NK cells have been engineered to express a chimeric receptor. In some embodiments, the expanding is ex vivo or in vitro. In some embodiments, the expanding is ex vivo. In some embodiments, the expanding is in vitro.

Provided herein is a method of enriching for educated natural killer (NK) cells from a biological sample, comprising, from a biological sample comprising NK cells, enriching for cells that express an inhibitory Killer Cell Ig-like Receptor (iKIR).

Also provided herein is a method of increasing the expansion of natural killer cells, the method comprising: from a biological sample comprising NK cells, enriching for cells that express an inhibitor Killer Cell Ig-like Receptor (iKIR).

In several embodiments, the iKIR is selected from among the group consisting of: KIR2DL1, KIR2DL2, KIR2DL3, KIR3DL1, KIR3DL2, KIR3DL3, KIR2DL5, and any combination thereof. In several embodiments, the method comprises enriching for cells that do not express NKG2A (NKG2A−). In several embodiments, the enriching thereby generates an enriched population comprising educated NK cells. In several embodiments, the method comprises enriching for cells that both (i) express an iKIR selected from among the group consisting of: KIR2DL1, KIR2DL2, KIR2DL3, KIR3DL1, KIR3DL2, KIR3DL3, KIR2DL5, and any combination thereof (iKIR+); and (ii) do not express NKG2A (NKG2A−).

Also provided herein is a method of enriching for educated natural killer (NK) cells from a biological sample, comprising, from a biological sample comprising NK cells, enriching for cells that both (i) express an inhibitory Killer Cell Ig-like Receptor (iKIR); and (ii) do not express NKG2A (NKG2A−). In several embodiments, the iKIR is selected from among the group consisting of: KIR2DL1, KIR2DL2, KIR2DL3, KIR3DL1, KIR3DL2, KIR3DL3, KIR2DL5, and any combination thereof. In several embodiments, the enriching thereby generates an enriched population comprising educated NK cells.

In several embodiments, the iKIR comprises KIR2DL1. In several embodiments, the iKIR comprises KIR2DL2. In several embodiments, the iKIR comprises KIR2DL3. In several embodiments, the iKIR comprises KIR3DL1. In several embodiments, the iKIR comprises KIR3DL2. In several embodiments, the iKIR comprises KIR3DL3. In several embodiments, the iKIR comprises KIR2DL5.

In several embodiments, the enriching for cells that express an iKIR comprises enriching for cells that express one iKIR, two iKIRs, or three iKIRs. In several embodiments, the enriching for cells that express an iKIR comprises enriching for cells that express one iKIR. In several embodiments, the enriching for cells that express an iKIR comprises enriching for cells that express two iKIRs. In several embodiments, the enriching for cells that express an iKIR comprises enriching for cells that express three iKIRs. In several embodiments, the enriching for cells that express an iKIR comprises enriching for cells that express four iKIRs. In several embodiments, the enriching for cells that express an iKIR comprises enriching for cells that express five iKIRs. In several embodiments, the enriching for cells that express an iKIR comprises enriching for cells that express six iKIRs. In several embodiments, the enriching for cells that express an iKIR comprises enriching for cells that express seven iKIRs.

In several embodiments, at least about 25%, at least about 30%, at least about 35%, at least about 40%, at least about 45%, or at least about 50% of cells in the enriched population comprise NK cells that express an iKIR (iKIR+ cells). In several embodiments, at least about 25% of cells in the enriched population comprise NK cells that express an iKIR (iKIR+ cells). In several embodiments, at least about 30% of cells in the enriched population comprise NK cells that express an iKIR (iKIR+ cells). In several embodiments, at least about 35% of cells in the enriched population comprise NK cells that express an iKIR (iKIR+ cells). In several embodiments, at least about 40% of cells in the enriched population comprise NK cells that express an iKIR (iKIR+ cells). In several embodiments, at least about 45% of cells in the enriched population comprise NK cells that express an iKIR (iKIR+ cells). In several embodiments, at least about 50% of cells in the enriched population comprise NK cells that express an iKIR (iKIR+ cells).

In several embodiments at least about 10%, at least about 15%, at least about 20%, at least about 25%, or at least about 30% of cells in the enriched population comprise NK cells that both express an iKIR and do not express NKG2A (iKIR+NKG2A− cells). In several embodiments, at least about 10% of cells in the enriched population comprise NK cells that both express an iKIR and do not express NKG2A (iKIR+NKG2A− cells). In several embodiments, at least about 15% of cells in the enriched population comprise NK cells that both express an iKIR and do not express NKG2A (iKIR+NKG2A− cells). In several embodiments, at least about 20% of cells in the enriched population comprise NK cells that both express an iKIR and do not express NKG2A (iKIR+NKG2A− cells). In several embodiments, at least about 25% of cells in the enriched population comprise NK cells that both express an iKIR and do not express NKG2A (iKIR+NKG2A− cells). In several embodiments, at least about 30% of cells in the enriched population comprise NK cells that both express an iKIR and do not express NKG2A (iKIR+NKG2A− cells).

In several embodiments, the iKIR+ cells express CD57. In several embodiments, the iKIR+ cells do not express CD57. In several embodiments, the iKIR+NKG2A− cells express CD57. In several embodiments, the iKIR+NKG2A− cells do not express CD57. In several embodiments, the iKIR+ cells express NKG2C. In several embodiments, the iKIR+ cells do not express NKG2C. In several embodiments, the iKIR+NKG2A− cells express NKG2C. In several embodiments, the iKIR+NKG2A− cells do not express NKG2C.

In several embodiments, the method comprises obtaining the biological sample from a donor. In several embodiments, the biological sample is a blood sample. In several embodiments, the blood sample is a peripheral blood (PB) sample. In several embodiments, the blood sample is a cord blood (CB) sample.

In several embodiments, enriching for cells that express an iKIR comprises positive selection. In several embodiments, enriching for cells that express an iKIR comprises contacting the NK cells with an antibody that binds to iKIR. In several embodiments, enriching for cells that do not express NKG2A comprises negative selection. In several embodiments, enriching for cells that do not express NKG2A comprises contacting the NK cells with an antibody that binds to NKG2A.

In several embodiments, the method comprises expanding the enriched population comprising educated NK cells, thereby generating an expanded NK cell population. In several embodiments, expanding the enriched population comprising educated NK cells comprises co-culturing NK cells of the enriched population with a feeder cell population. In some embodiments, the co-culturing is carried out in a culture media comprising (e.g., supplemented with) soluble interleukin 12 (IL12) and/or soluble interleukin 18 (IL18), In some embodiments, the culture media comprises (e.g., is supplemented with) IL12. In some embodiments, the culture media comprises (e.g., is supplemented with) IL18. In some embodiments, the culture media comprises (e.g., is supplemented with) IL12 and IL18. In several embodiments, expanding the enriched population comprising educated NK cells comprises co-culturing, in a culture media supplemented with soluble interleukin 12 (IL12) and soluble interleukin 18 (IL18), NK cells of the enriched population with a feeder cell population. In several embodiments, the feeder cell population comprises cells engineered to express 4-1BBL. In several embodiments, the feeder cell population comprises cells engineered to express membrane-bound interleukin-15 (mbIL15). In several embodiments, the feeder cell population comprises cells engineered to express 4-1BBL and mbIL15. In several embodiments, the ratio of NK cells to feeder cells ranges from about 1:2 to about 1:10. In several embodiments, the ratio of NK cells to feeder cells is from about 1:3 to about 1:5. In several embodiments, the ratio of NK cells to feeder cells is about 1:3. In several embodiments, the ratio of NK cells to feeder cells is about 1:4. In several embodiments, the ratio of NK cells to feeder cells is about 1:5. In some embodiments, the feeder cells comprise K562 cells.

In several embodiments, expanding the enriched population comprising educated NK cells comprises co-culturing, for a first time in a culture media, NK cells of the enriched population with a first batch of a feeder cell population. In some embodiments, the culture media comprises interleukin 12 (IL12). In some embodiments, the culture media comprises interleukin 18 (IL18). In some embodiments, the culture media comprises interleukin 2 (IL2). In some embodiments, the culture media comprises IL12 and IL18. In some embodiments, the culture media comprises IL2, IL12, and IL18. In some embodiments, the culture media comprises soluble interleukin 12 (IL12) and soluble interleukin 18 (IL18). In some embodiments, the culture media comprises soluble IL12, soluble IL12, and soluble IL18. In several embodiments, expanding the enriched population comprising educated NK cells comprises co-culturing, in a culture media, NK cells from the first co-culturing with a second batch of the feeder cell population, thereby generating a second co-culturing. In several embodiments, expanding the enriched population comprising educated NK cells comprises co-culturing, in a culture media, NK cells from the second co-culturing with a third batch of the feeder cell population, thereby generating a third co-culturing. In several embodiments, expanding the enriched population comprising educated NK cells comprises co-culturing, in a culture media, NK cells from the third co-culturing with a fourth batch of the feeder cell population, thereby generating a fourth co-culturing. In several embodiments, expanding the enriched population comprising educated NK cells comprises co-culturing, in a culture media, NK cells from the fourth co-culturing with a fifth batch of the feeder cell population, thereby generating a fifth co-culturing.

In several embodiments, expanding the enriched population comprising educated NK cells comprises co-culturing, for a first time, in a culture media supplemented with at least soluble interleukin 12 (IL12) and soluble interleukin 18 (IL18), NK cells of the enriched population with a first batch of a feeder cell population, co-culturing, in a culture media, NK cells from the first co-culturing with a second batch of the feeder cell population, thereby generating a second co-culturing, co-culturing, in a culture media, NK cells from the second co-culturing with a third batch of the feeder cell population, thereby generating a third co-culturing, co-culturing, in the culture media, NK cells from the third co-culturing with a fourth batch of the feeder cell population, thereby generating a fourth co-culturing, and co-culturing, for a fifth time, in a culture media again supplemented with at least soluble IL12 and soluble IL18, NK cells from the fourth co-culturing with a fifth batch of the feeder cell population, thereby generating a fifth co-culturing, and resulting in a population of expanded NK cells. In several embodiments, the feeder cell population comprises cells engineered to express 4-1BBL and membrane-bound interleukin-15 (mbIL15).

In several embodiments, a ratio of NK cells to feeder cells at each co-culturing ranges from about 1:2 to about 1:10. In several embodiments, the ratio of NK cells to feeder cells at each co-culturing ranges from about 1:3 to about 1:5. In several embodiments, the ratio of NK cells to feeder cells at each co-culturing is about 1:3. In several embodiments, the ratio of NK cells to feeder cells at each co-culturing is about 1:4. In several embodiments, the ratio of NK cells to feeder cells at each co-culturing is about 1:5. Other ratios are used in other embodiments, such as about 1:1, 1:4, 1:20, 1:50, 50:1, 25:1, 15:1, 10:1, 2:1 etc.

In several embodiments, the IL12 is present in the supplemented media at a concentration ranging from about 0.01 ng/ml to about 10 ng/ml (or at an equivalent concentration using other units of concentration, e.g., IU/mL). In several embodiments, the IL12 is present in the supplemented media at a concentration of about 0.25 ng/ml (or at an equivalent concentration using other units of concentration, e.g., IU/mL). In several embodiments, the IL12 is present in the supplemented media at a concentration of about 0.5 ng/ml (or at an equivalent concentration using other units of concentration, e.g., IU/mL). In several embodiments, the IL12 is present in the supplemented media at a concentration of about 1 ng/ml (or at an equivalent concentration using other units of concentration, e.g., IU/mL). In several embodiments, the IL12 is present in the supplemented media at a concentration of about 5 ng/ml (or at an equivalent concentration using other units of concentration, e.g., IU/mL). In several embodiments, the IL18 is present in the supplemented media at a concentration ranging from about 10 ng/ml to about 30 ng/ml (or at an equivalent concentration using other units of concentration, e.g., IU/mL). In several embodiments, the IL18 is present in the supplemented media at a concentration of about 10 ng/ml (or at an equivalent concentration using other units of concentration, e.g., IU/mL). In several embodiments, the IL18 is present in the supplemented media at a concentration of about 20 ng/ml (or at an equivalent concentration using other units of concentration, e.g., IU/mL). In several embodiments, the IL18 is present in the supplemented media at a concentration of about 30 ng/ml (or at an equivalent concentration using other units of concentration, e.g., IU/mL). In several embodiments, one or more of the co-culturings employs media supplemented with soluble IL2. In several embodiments, the IL2 is present in the supplemented media at a concentration ranging from about 25 to about 50 units/mL (or at an equivalent concentration using other units of concentration, e.g., ng/ml). In several embodiments, the IL2 is present in the supplemented media at a concentration ranging of about 30 units/mL (or at an equivalent concentration using other units of concentration, e.g., ng/ml). In several embodiments, the IL2 is present in the supplemented media at a concentration ranging of about 40 units/mL (or at an equivalent concentration using other units of concentration, e.g., ng/ml). In several embodiments, the IL2 is present in the supplemented media at a concentration ranging of about 50 units/mL (or at an equivalent concentration using other units of concentration, e.g., ng/ml). In several embodiments, the IL2 is present in the supplemented media for at least the first and the fifth co-culturing.

In several embodiments, expanding the enriched population comprising educated NK cells does not comprise contacting NK cells with a stimulator of NKG2A. In some embodiments, expanding the enriched population comprising educated NK cells does not comprise contacting NK cells with a stimulator of NKG2C. In several embodiments, expanding the enriched population comprising educated NK cells does not comprise contacting NK cells with a stimulator of NKG2A or NKG2C. In several embodiments, expanding the enriched population comprising educated NK cells does not comprise contacting NK cells with HLA-E. In several embodiments, the feeder cells of the feeder cell population do not express HLA-E.

In several embodiments, the NK cells are frozen (e.g., cryopreserved) after co-culturing. In several embodiments, the NK cells are frozen (e.g., cryopreserved) after a given co-culturing and thawed prior to the subsequent co-culturing. In several embodiments, the NK cells are frozen at least two times between the first and the fifth co-culturing.

In several embodiments, the methods comprise genetically editing NK cells of the biological sample to reduce or eliminate expression of at least one endogenous gene or protein expressed as compared to a non-modified NK cell. In several embodiments, the methods comprise genetically editing NK cells of the expanded NK cell population to reduce or eliminate expression of at least one endogenous gene or protein expressed as compared to a non-modified NK cell. In some embodiments, NK cells of the biological sample are genetically edited to reduce or eliminate expression of at least one endogenous gene or protein expressed as compared to a non-modified NK cell. In some embodiments, NK cells of the expanded NK cell population are genetically edited to reduce or eliminate expression of at least one endogenous gene or protein expressed as compared to a non-modified NK cell. In some embodiments, the genetic editing thereby generates a genetically edited NK cell population.

In several embodiments, the methods further comprise genetically modifying (e.g., genetically editing) the NK cells to reduce or eliminate expression of at least one endogenous gene or protein expressed as compared to a non-modified NK cell, wherein the genetic modification is performed prior to the first or second co-culturing. In some embodiments, the methods further comprise genetically editing the NK cells to reduce or eliminate expression of at least one endogenous gene or protein expressed as compared to a non-modified NK cell. In some embodiments, the genetic editing is performed prior to the first or second co-culturing. In some embodiments, the genetic editing is performed prior to the first co-culturing. In some embodiments, the genetic editing is performed prior to the second co-culturing.

In some embodiments, the endogenous gene comprises the cytokine-inducible SH2-containing protein (CISH) gene. In several embodiments, the genetic modification comprises a disruption of the cytokine-inducible SH2-containing protein (CISH) gene. In several embodiments, disruption of the CISH gene thereby results in reduced or eliminated CIS expression by the NK cell. In several embodiments, disruption of the CISH gene reduces or eliminates CISH gene expression. In several embodiments, disruption of the CISH gene reduces CISH gene expression. In several embodiments, disruption of the CISH gene eliminates CISH gene expression. In several embodiments, disruption of the CISH gene reduces or eliminates CISH protein expression. In several embodiments, disruption of the CISH gene reduces CISH protein expression. In several embodiments, disruption of the CISH gene eliminates CISH protein expression. Other genes disclosed herein may also be edited, alone, or in combination with CISH.

In some embodiments, the endogenous gene comprises the Casitas B-Lineage Lymphoma Proto-Oncogene B (CBLB) gene. In several embodiments, the genetic modification comprises a disruption of the CBLB gene. In several embodiments, disruption of the CBLB gene reduces or eliminates CBLB gene expression. In several embodiments, disruption of the CBLB gene reduces CBLB gene expression. In several embodiments, disruption of the CBLB gene eliminates CBLB gene expression. In several embodiments, disruption of the CBLB gene reduces or eliminates CBLB protein expression. In several embodiments, disruption of the CBLB gene reduces CBLB protein expression. In several embodiments, disruption of the CBLB gene eliminates CBLB protein expression.

In some embodiments, the endogenous gene comprises the Mediator Complex Subunit 12 (MED12) gene. In several embodiments, the genetic modification comprises a disruption of the MED12 gene. In several embodiments, disruption of the MED12 gene reduces or eliminates MED12 gene expression. In several embodiments, disruption of the MED12 gene reduces MED12 gene expression. In several embodiments, disruption of the MED12 gene eliminates MED12 gene expression. In several embodiments, disruption of the MED12 gene reduces or eliminates MED12 protein expression. In several embodiments, disruption of the MED12 gene reduces MED12 protein expression. In several embodiments, disruption of the MED12 gene eliminates MED12 protein expression.

In some embodiments, the endogenous gene comprises CISH and CBLB. In some embodiments, the endogenous gene comprises CISH and MED12. In some embodiments, the endogenous gene comprises CBLB and MED12. In some embodiments, the endogenous gene comprises CISH, CBLB, and MED12.

In several embodiments, the method comprises engineering the NK cells to express a chimeric antigen receptor (CAR) that binds to a tumor antigen. In several embodiments, the method comprises engineering NK cells of the expanded NK cell population to express a chimeric antigen receptor (CAR) that binds a tumor antigen In several embodiments, the method comprises engineering NK cells of the genetically edited NK cell population to express a chimeric antigen receptor (CAR) that binds a tumor antigen. In several embodiments, NK cells of the expanded NK cell population are engineered to express a chimeric antigen receptor (CAR) that binds to a tumor antigen. In several embodiments, NK cells of the genetically edited NK cell population are engineered to express a chimeric antigen receptor (CAR) that binds to a tumor antigen. In several embodiments, the engineering thereby generates an engineered NK cell population.

In several embodiments, the methods further comprise engineering the NK cells to express a chimeric antigen receptor that is directed against a tumor target and promotes cytotoxic activity against a tumor cell expressing the tumor target. In several embodiments, the methods further comprise engineering the NK cells to express a chimeric antigen receptor (CAR). In some embodiments, the CAR binds to an antigen expressed by target cells. In some embodiments, the target cells comprise cancer cells. In some embodiments, the target cells comprise B cells. In some embodiments, the target cells comprise T cells. In some embodiments, the target cells comprise plasma cells.

Also provided herein is a method of producing a population of engineered natural killer (NK) cells, the method comprising: (a) from a biological sample comprising NK cells, enriching for cells that express an inhibitor Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), the enriching thereby generating an enriched population comprising educated NK cells; (b) expanding the enriched population comprising educated NK cells, thereby generating an expanded NK cell population; and (c) engineering NK cells of the expanded NK cell population to express a chimeric antigen receptor (CAR) that binds an antigen, thereby generating an engineered NK cell population.

In several embodiments, the tumor target is selected from a ligand for the NKG2D receptor, CD19, CD70, BCMA, or CD38. In several embodiments, the tumor target is a ligand for the NKG2D receptor. In several embodiments, the tumor target is CD19. In several embodiments, the tumor target is CD70. In several embodiments, the tumor target is BCMA. In several embodiments, the tumor target is CD38. In several embodiments, the antigen is selected from a ligand for the NKG2D receptor, CD19, CD70, BCMA, or CD38. In several embodiments, the antigen is a ligand for the NKG2D receptor. In several embodiments, the antigen is CD19. In several embodiments, the antigen is CD70. In several embodiments, the antigen is BCMA. In several embodiments, the antigen is CD38.

In several embodiments, the CAR comprises a transmembrane domain. In several embodiments, the CAR comprises an intracellular signaling region. In several embodiments, the CAR comprises a transmembrane domain and an intracellular signaling region. In several embodiments, the transmembrane domain comprises CD8, CD28, or a portion thereof. In several embodiments, the transmembrane domain comprises CD8 or a portion thereof. In several embodiments, the transmembrane domain comprises CD28 or a portion thereof. In several embodiments, the transmembrane domain comprises CD8 alpha (CD8a). In several embodiments, the intracellular signaling region comprises a CD3zeta domain. In several embodiments, the intracellular signaling region comprises an intracellular signaling domain of an OX40, 4-1BB, CD28, or a signaling portion thereof. In several embodiments, the intracellular signaling region comprises an intracellular signaling domain of OX40. In several embodiments, the intracellular signaling region comprises an intracellular signaling domain of 4-1BB. In several embodiments, the intracellular signaling region comprises an intracellular signaling domain of CD28.

In several embodiments, the method comprises engineering NK cells of the expanded NK cell population, the genetically edited NK cell population, or the engineered NK cell population to express membrane-bound interleukin 15 (mbIL15). In several embodiments, the method comprises engineering NK cells of the expanded NK cell population to express mbIL15. In several embodiments, the method comprises engineering NK cells of the genetically edited NK cell population to express mbIL15. In several embodiments, the method comprises engineering NK cells of the engineered NK cell population to express mbIL15. In several embodiments, NK cells of the expanded NK cell population, the genetically edited NK cell population, or the engineered NK cell population are engineered to express membrane-bound interleukin 15 (mbIL15). In several embodiments, NK cells of the expanded NK cell population are engineered to express mbIL15. In several embodiments, NK cells of the genetically edited NK cell population are engineered to express mbIL15. In several embodiments, NK cells of the engineered NK cell population are engineered to express mbIL15.

In several embodiments, the engineering of the NK cells is concurrent with or after the genetic editing. In several embodiments, the engineering of the NK cells is concurrent with the genetic editing. In several embodiments, the engineering of the NK cells is after the genetic editing.

Also provided herein is a population of NK cells produced by any of the methods disclosed herein. Also provided for herein are uses of a population of NK cells produced by any of the methods disclosed herein for the treatment of a disease or condition in a subject. In some embodiments, the disease or condition is an autoimmune disease. In some embodiments, the disease or condition is cancer.

Also provided for herein are uses of a population of NK cells produced by any of the methods disclosed herein for the treatment of an autoimmune disease in a subject. Additionally provided for herein are uses of a population of NK cells produced by any of the methods disclosed herein in the preparation of a medicament for the treatment of an autoimmune disease in a subject. Also provided for herein are methods of treating an autoimmune disease in a subject, comprising administering to a subject a population of NK cells produced by any of the methods disclosed herein. In several embodiments, the population of NK cells is allogeneic to the subject.

In some embodiments, the autoimmune disease is a B cell-mediated autoimmune disease. In some embodiments, the autoimmune disease is a T cell-mediated autoimmune disease. In some embodiments, the autoimmune disease is a plasma cell-mediated autoimmune disease. In some embodiments, the autoimmune disease is systemic lupus erythematosus (SLE), lupus nephritis (LN), scleroderma (e.g., systemic sclerosis), vasculitis (e.g., ANCA vasculitis), myositis, antisynthetase syndrome, rheumatoid arthritis (RA), or multiple sclerosis (MS). In some embodiments, the autoimmune disease is SLE. In some embodiments, the autoimmune disease is LN. In some embodiments, the autoimmune disease is scleroderma. In some embodiments, the autoimmune disease is systemic sclerosis. In some embodiments, the autoimmune disease is vasculitis. In some embodiments, the autoimmune disease is ANCA vasculitis. In some embodiments, the autoimmune disease is myositis. In some embodiments, the autoimmune disease is antisynthetase syndrome. In some embodiments, the autoimmune disease is RA. In some embodiments, the autoimmune disease is MS.

In several embodiments, the CAR binds to an antigen expressed by B cells. In several embodiments, the CAR binds to an antigen not expressed, or no longer expressed, by B cells. In several embodiments, the CAR binds to an antigen expressed by T cells. In several embodiments, the CAR binds to an antigen not expressed, or no longer expressed, by T cells. In several embodiments, the CAR binds to an antigen expressed by plasma cells. In several embodiments, the CAR binds to an antigen not expressed, or no longer expressed, by plasma cells.

Also provided for herein are uses of a population of NK cells produced by any of the methods disclosed herein for the treatment of a disease or condition in a subject. In some embodiments, the disease or condition is an autoimmune disease. In some embodiments, the disease or condition is a cancer.

Also provided for herein are uses of a population of NK cells produced by any of the methods disclosed herein for the treatment of cancer in a subject.

Also provided for herein are uses of a population of NK cells produced by any of the methods disclosed herein in the preparation of a medicament for the treatment of a disease or condition in a subject. In some embodiments, the disease or condition is an autoimmune disease. In some embodiments, the disease or condition is a cancer. Additionally provided for herein are uses of a population of NK cells produced by any of the methods disclosed herein in the preparation of a medicament for the treatment of cancer in a subject.

Also provided for herein are methods of treating a disease or condition in a subject, comprising administering to a subject a population of NK cells produced by any of the methods disclosed herein. In some embodiments, the disease or condition is an autoimmune disease. In some embodiments, the disease or condition is a cancer.

Also provided for herein are methods of treating a cancer in a subject, comprising administering to a subject a population of NK cells produced by any of the methods disclosed herein.

In several embodiments, the population of NK cells is allogeneic to the subject.

In some embodiments, the autoimmune disease is a B cell-mediated autoimmune disease. In some embodiments, the autoimmune disease is a T cell-mediated autoimmune disease. In some embodiments, the autoimmune disease is a plasma cell-mediated autoimmune disease. In some embodiments, the autoimmune disease is systemic lupus erythematosus (SLE), lupus nephritis (LN), scleroderma (e.g., systemic sclerosis), vasculitis (e.g., ANCA vasculitis), myositis, antisynthetase syndrome, rheumatoid arthritis (RA), or multiple sclerosis (MS).

In several embodiments, the CAR binds to an antigen expressed by B cells. In several embodiments, the CAR binds to an antigen expressed by T cells. In several embodiments, the CAR binds to an antigen expressed by plasma cells.

In several embodiments, the cancer is a hematologic malignancy. In some embodiments, the cancer is a leukemia or a lymphoma. In several embodiments, the cancer is a leukemia. In several embodiments, the cancer is a lymphoma. In some embodiments, the cancer is multiple myeloma. In some embodiments, the cancer is or comprises a solid tumor.

In several embodiments, the CAR binds to an antigen expressed by the cancer. In several embodiments, the CAR binds to an antigen not expressed, or no longer expressed, by cells of the cancer.

In several embodiments, the method further comprises assessing the cytomegalovirus (CMV) status of the biological sample. In several embodiments, the biological sample is from a subject who is cytomegalovirus (CMV) positive (CMV+).

In several embodiments, the method further comprises detecting the degree of Human Leukocyte Antigen (HLA) mismatch between the biological sample and a target tumor cell by determining the number of iKIR triggered by tumor HLA.

In several embodiments, a population of NK cells produced by any of the methods described herein exhibits increased natural cytotoxicity against target cells (e.g., not CAR-mediated cytotoxicity), as compared to a population of NK cells produced by a method not comprising enrichment for iKIR+ cells.

Also provided herein is a method for selecting a donor of natural killer (NK) cells for immunotherapy, comprising: (a) assessing in a biological sample from a donor, the percentage of cells expressing an inhibitory Killer Cell Ig-like Receptor (iKIR+), wherein the biological sample comprises NK cells; (b) determining whether the percentage of iKIR+ cells in the biological sample is at or above a threshold value, wherein the threshold value is at least about 25%; and (c) if the percentage of iKIR+ cells is at or above the threshold value, selecting the biological sample for use in immunotherapy; or if the percentage of iKIR+ cells is less than the threshold value, not selecting the biological sample for use in immunotherapy. In several embodiments, an iKIR is selected from among the group consisting of: KIR2DL1, KIR2DL2, KIR2DL3, KIR3DL1, KIR3DL2, KIR3DL3, KIR2DL5, or any combination thereof. In several embodiments, the threshold value is about 25%, about 30%, about 35%, about 40%, about 45%, or about 50%. In several embodiments, the threshold value is about 25%. In several embodiments, the threshold value is about 30%. In several embodiments, the threshold value is about 35%. In several embodiments, the threshold value is about 40%. In several embodiments, the threshold value is about 45%. In several embodiments, the threshold value is about 50%.

Also provided herein is a method comprising: (a) assessing in a biological sample from a donor, the percentage of cells that both (i) express an inhibitory Killer Cell Ig-like Receptor (iKIR+); and (ii) do not express NKG2A (NKG2A−), wherein the biological sample comprises NK cells; (b) determining whether the percentage of iKIR+NKG2A− cells in the biological sample is at or above a threshold value, wherein the threshold value is at least about 10%; and (c) if the percentage of iKIR+NKG2A− cells is at or above the threshold value, selecting the biological sample for use in immunotherapy; or if the percentage of iKIR+NKG2A− cells is less than the threshold value, not selecting the biological sample for use in immunotherapy. In several embodiments, the iKIR is selected from among the group consisting of: KIR2DL1, KIR2DL2, KIR2DL3, KIR3DL1, KIR3DL2, KIR3DL3, KIR2DL5, or any combination thereof. In several embodiments, the threshold value is about 10%, about 15%, about 20%, about 25%, or about 30%. In several embodiments, the threshold value is about 10%. In several embodiments, the threshold value is about 15%. In several embodiments, the threshold value is about 20%. In several embodiments, the threshold value is about 25%. In several embodiments, the threshold value is about 30%.

In several embodiments, the iKIR comprises KIR2DL1. In several embodiments, the iKIR comprises KIR2DL2. In several embodiments, the iKIR comprises KIR2DL3. In several embodiments, the iKIR comprises KIR3DL1. In several embodiments, the iKIR comprises KIR3DL2. In several embodiments, the iKIR comprises KIR3DL3. In several embodiments, the iKIR comprises KIR2DL5.

In several embodiments, the iKIR+ cells express CD57. In several embodiments, the iKIR+ cells do not express CD57. In several embodiments, the iKIR+NKG2A− cells express CD57. In several embodiments, the iKIR+NKG2A− cells do not express CD57. In several embodiments, the iKIR+ cells do not express NKG2C. In several embodiments, the iKIR+NKG2A− cells do not express NKG2C.

In some embodiments, cells that express an iKIR express one iKIR, two iKIRs, or three iKIRs. In some embodiments, cells that express an iKIR express one iKIR. In some embodiments, cells that express an iKIR express two iKIRs. In some embodiments, cells that express an iKIR express three iKIRs. In some embodiments, cells that express an iKIR express four iKIRs. In some embodiments, cells that express an iKIR express five iKIRs. In some embodiments, cells that express an iKIR express six iKIRs. In some embodiments, cells that express an iKIR express seven iKIRs.

In several embodiments, the method comprises obtaining the biological sample from the donor. In several embodiments, the method comprises assessing the cytomegalovirus (CMV) status of the immune cells from a donor. In several embodiments, the donor is cytomegalovirus (CMV) positive (CMV+).

In several embodiments, the biological sample is a blood sample. In several embodiments, the blood sample is a peripheral blood sample. In several embodiments, the blood sample is a cord blood sample.

In several embodiments, if the biological sample is selected for use in immunotherapy, the method comprises expanding the NK cells of the biological sample. In several embodiments, the expanding thereby generates an expanded NK cell population. In several embodiments, expanding the enriched population comprising educated NK cells comprises co-culturing NK cells of the enriched population with a feeder cell population. In some embodiments, the co-culturing is carried out in a culture media comprising (e.g., supplemented with) soluble interleukin 12 (IL12) and/or soluble interleukin 18 (IL18), In some embodiments, the culture media comprises (e.g., is supplemented with) IL12. In some embodiments, the culture media comprises (e.g., is supplemented with) IL18. In some embodiments, the culture media comprises (e.g., is supplemented with) Interleukin 2 (IL2). In some embodiments, the culture media comprises (e.g., is supplemented with) IL12 and IL18. In some embodiments, the culture media comprises (e.g., is supplemented with) IL2, IL12, and IL18. In several embodiments, expanding the NK cells of the biological sample comprises co-culturing, in a culture media supplemented with soluble interleukin 12 (IL12) and soluble interleukin 18 (IL18), NK cells of biological sample with a feeder cell population. In several embodiments, the feeder cell population comprises cells engineered to express 4-1BBL. In several embodiments, the feeder cell population comprises cells engineered to express membrane-bound interleukin-15 (mbIL15). In several embodiments, the feeder cell population comprises cells engineered to express 4-1BBL and membrane-bound interleukin-15 (mbIL15). In several embodiments, the ratio of NK cells to feeder cells ranges from about 1:2 to about 1:10. In several embodiments, the ratio of NK cells to feeder cells is from about 1:3 to about 1:5. In several embodiments, the ratio of NK cells to feeder cells is about 1:3. In several embodiments, the ratio of NK cells to feeder cells is about 1:4. In several embodiments, the ratio of NK cells to feeder cells is about 1:5. In some embodiments, the feeder cells comprise K562 cells.

In several embodiments, expanding the enriched population comprising educated NK cells comprises co-culturing, for a first time in a culture media, NK cells of the enriched population with a first batch of a feeder cell population. In some embodiments, the culture media comprises interleukin 12 (IL12). In some embodiments, the culture media comprises interleukin 18 (IL18). In some embodiments, the culture media comprises interleukin 2 (IL2). In some embodiments, the culture media comprises IL12 and IL18. In some embodiments, the culture media comprises IL2, IL12, and IL18. In some embodiments, the culture media comprises soluble interleukin 12 (IL12) and soluble interleukin 18 (IL18). In some embodiments, the culture media comprises soluble IL12, soluble IL12, and soluble IL18. In several embodiments, expanding the enriched population comprising educated NK cells comprises co-culturing, in a culture media, NK cells from the first co-culturing with a second batch of the feeder cell population, thereby generating a second co-culturing. In several embodiments, expanding the enriched population comprising educated NK cells comprises co-culturing, in a culture media, NK cells from the second co-culturing with a third batch of the feeder cell population, thereby generating a third co-culturing. In several embodiments, expanding the enriched population comprising educated NK cells comprises co-culturing, in a culture media, NK cells from the third co-culturing with a fourth batch of the feeder cell population, thereby generating a fourth co-culturing. In several embodiments, expanding the enriched population comprising educated NK cells comprises co-culturing, in a culture media, NK cells from the fourth co-culturing with a fifth batch of the feeder cell population, thereby generating a fifth co-culturing.

In several embodiments, expanding the NK cells of the biological sample comprises co-culturing, for a first time, in a culture media supplemented with at least soluble interleukin 12 (IL12) and soluble interleukin 18 (IL18), NK cells of the biological sample with a first batch of a feeder cell population, co-culturing, in a culture media, NK cells from the first co-culturing with a second batch of the feeder cell population, thereby generating a second co-culturing, co-culturing, in a culture media, NK cells from the second co-culturing with a third batch of the feeder cell population, thereby generating a third co-culturing, co-culturing, in the culture media, NK cells from the third co-culturing with a fourth batch of the feeder cell population, thereby generating a fourth co-culturing, and co-culturing, for a fifth time, in a culture media again supplemented with at least soluble IL12 and soluble IL18, NK cells from the fourth co-culturing with a fifth batch of the feeder cell population, thereby generating a fifth co-culturing, and resulting in a population of expanded NK cells. In several embodiments, the feeder cell population comprises cells engineered to express 4-1BBL and membrane-bound interleukin-15 (mbIL15).

In several embodiments, a ratio of NK cells to feeder cells at each co-culturing ranges from about 1:2 to about 1:10. In several embodiments, the ratio of NK cells to feeder cells at each co-culturing ranges from about 1:3 to about 1:5. In several embodiments, the ratio of NK cells to feeder cells is about 1:3. In several embodiments, the ratio of NK cells to feeder cells is about 1:4. In several embodiments, the ratio of NK cells to feeder cells is about 1:5. Other ratios are used in other embodiments, such as about 1:1, 1:4, 1:20, 1:50, 50:1, 25:1, 15:1, 10:1, 2:1 etc.

In several embodiments, the IL12 is present in the supplemented media at a concentration ranging from about 0.01 ng/ml to about 10 ng/ml (or at an equivalent concentration using other units of concentration, e.g., IU/mL). In several embodiments, the IL12 is present in the supplemented media at a concentration of about 0.25 ng/ml (or at an equivalent concentration using other units of concentration, e.g., IU/mL). In several embodiments, the IL12 is present in the supplemented media at a concentration of about 0.5 ng/ml (or at an equivalent concentration using other units of concentration, e.g., IU/mL). In several embodiments, the IL12 is present in the supplemented media at a concentration of about 1 ng/ml (or at an equivalent concentration using other units of concentration, e.g., IU/mL). In several embodiments, the IL12 is present in the supplemented media at a concentration of about 5 ng/ml (or at an equivalent concentration using other units of concentration, e.g., IU/mL). In several embodiments, the IL18 is present in the supplemented media at a concentration ranging from about 10 ng/ml to about 30 ng/ml (or at an equivalent concentration using other units of concentration, e.g., IU/mL). In several embodiments, the IL18 is present in the supplemented media at a concentration of about 10 ng/ml (or at an equivalent concentration using other units of concentration, e.g., IU/mL). In several embodiments, the IL18 is present in the supplemented media at a concentration of about 20 ng/mL (or at an equivalent concentration using other units of concentration, e.g., IU/mL). In several embodiments, the IL18 is present in the supplemented media at a concentration of about 30 ng/ml (or at an equivalent concentration using other units of concentration, e.g., IU/mL). In several embodiments, one or more of the co-culturings employs media supplemented with soluble IL2. In several embodiments, the IL2 is present in the supplemented media at a concentration ranging from about 25 to about 50 units/mL (or at an equivalent concentration using other units of concentration, e.g., ng/ml). In several embodiments, the IL2 is present in the supplemented media at a concentration ranging of about 30 units/mL (or at an equivalent concentration using other units of concentration, e.g., ng/ml). In several embodiments, the IL2 is present in the supplemented media at a concentration ranging of about 40 units/mL (or at an equivalent concentration using other units of concentration, e.g., ng/ml). In several embodiments, the IL2 is present in the supplemented media at a concentration ranging of about 50 units/mL (or at an equivalent concentration using other units of concentration, e.g., ng/ml). In several embodiments, the IL2 is present in the supplemented media for at least the first and the fifth co-culturing.

In several embodiments, expanding the NK cells of the biological sample does not comprise contacting NK cells with a stimulator of NKG2A. In some embodiments, expanding the NK cells of the biological sample does not comprise contacting NK cells with a stimulator of NKG2C. In several embodiments, expanding the NK cells of the biological sample does not comprise contacting NK cells with a stimulator of NKG2A or NKG2C. In several embodiments, expanding the NK cells of the biological sample does not comprise contacting NK cells with HLA-E. In several embodiments, the feeder cells of the feeder cell population do not express HLA-E.

In several embodiments, the NK cells are frozen (e.g., cryopreserved) after co-culturing. In several embodiments, the NK cells are frozen (e.g., cryopreserved) after a given co-culturing and thawed prior to the subsequent co-culturing. In several embodiments, the NK cells are frozen (e.g., cryopreserved) after the first co-culturing. In several embodiments, the NK cells are frozen (e.g., cryopreserved) after a first co-culturing and thawed prior to the second co-culturing. In several embodiments, the NK cells are frozen at least two times between the first and the fifth co-culturing.

In several embodiments, the methods comprise genetically editing NK cells of the biological sample to reduce or eliminate expression of at least one endogenous gene or protein expressed as compared to a non-modified NK cell. In several embodiments, the methods comprise genetically editing NK cells of the expanded NK cell population to reduce or eliminate expression of at least one endogenous gene or protein expressed as compared to a non-modified NK cell. In some embodiments, NK cells of the biological sample are genetically edited to reduce or eliminate expression of at least one endogenous gene or protein expressed as compared to a non-modified NK cell. In some embodiments, NK cells of the expanded NK cell population are genetically edited to reduce or eliminate expression of at least one endogenous gene or protein expressed as compared to a non-modified NK cell. In some embodiments, the genetic editing thereby generates a genetically edited NK cell population.

In several embodiments, the methods further comprise genetically modifying (e.g., genetically editing) the NK cells to reduce or eliminate expression of at least one endogenous gene or protein expressed as compared to a non-modified NK cell, wherein the genetic modification is performed prior to the first or second co-culturing.

In some embodiments, the endogenous gene comprises the cytokine-inducible SH2-containing protein (CISH) gene. In several embodiments, the genetic modification comprises a disruption of the cytokine-inducible SH2-containing protein (CISH) gene. In several embodiments, disruption of the CISH gene thereby results in reduced or eliminated CIS expression by the NK cell. In several embodiments, disruption of the CISH gene reduces or eliminates CISH gene expression. In several embodiments, disruption of the CISH gene reduces CISH gene expression. In several embodiments, disruption of the CISH gene eliminates CISH gene expression. In several embodiments, disruption of the CISH gene reduces or eliminates CISH protein expression. In several embodiments, disruption of the CISH gene reduces CISH protein expression. In several embodiments, disruption of the CISH gene eliminates CISH protein expression. Other genes disclosed herein may also be edited, alone, or in combination with CISH.

In some embodiments, the endogenous gene comprises the Casitas B-Lineage Lymphoma Proto-Oncogene B (CBLB) gene. In several embodiments, the genetic modification comprises a disruption of the CBLB gene. In several embodiments, disruption of the CBLB gene reduces or eliminates CBLB gene expression. In several embodiments, disruption of the CBLB gene reduces CBLB gene expression. In several embodiments, disruption of the CBLB gene eliminates CBLB gene expression. In several embodiments, disruption of the CBLB gene reduces or eliminates CBLB protein expression. In several embodiments, disruption of the CBLB gene reduces CBLB protein expression. In several embodiments, disruption of the CBLB gene eliminates CBLB protein expression.

In some embodiments, the endogenous gene comprises the Mediator Complex Subunit 12 (MED12) gene. In several embodiments, the genetic modification comprises a disruption of the MED12 gene. In several embodiments, disruption of the MED12 gene reduces or eliminates MED12 gene expression. In several embodiments, disruption of the MED12 gene reduces MED12 gene expression. In several embodiments, disruption of the MED12 gene eliminates MED12 gene expression. In several embodiments, disruption of the MED12 gene reduces or eliminates MED12 protein expression. In several embodiments, disruption of the MED12 gene reduces MED12 protein expression. In several embodiments, disruption of the MED12 gene eliminates MED12 protein expression.

In some embodiments, the endogenous gene comprises CISH and CBLB. In some embodiments, the endogenous gene comprises CISH and MED12. In some embodiments, the endogenous gene comprises CBLB and MED12. In some embodiments, the endogenous gene comprises CISH, CBLB, and MED12.

In several embodiments, the method comprises engineering NK cells of the expanded NK cell population to express a chimeric antigen receptor (CAR) that binds a tumor antigen. In several embodiments, the method comprises engineering NK cells of the genetically edited NK cell population to express a chimeric antigen receptor (CAR) that binds a tumor antigen. In several embodiments, NK cells of the expanded NK cell population are engineered to express a chimeric antigen receptor (CAR) that binds to a tumor antigen. In several embodiments, NK cells of the genetically edited NK cell population are engineered to express a chimeric antigen receptor (CAR) that binds to a tumor antigen.

In several embodiments, the method comprises engineering NK cells of the expanded NK cell population to express a CAR that binds an antigen expressed by target cells. In several embodiments, the method comprises engineering NK cells of the genetically edited NK cell population to express a CAR that binds an antigen expressed by target cells. In several embodiments, NK cells of the expanded NK cell population are engineered to express a CAR that binds to an antigen expressed by target cells. In several embodiments, NK cells of the genetically edited NK cell population are engineered to express a CAR that binds to an antigen expressed by target cells. In several embodiments, the engineering thereby generates an engineered NK cell population.

In several embodiments, the methods further comprise engineering the NK cells express a chimeric antigen receptor that is directed against a tumor target and promotes cytotoxic activity against a tumor cell expressing the tumor target. In several embodiments, the methods further comprise engineering the NK cells express a CAR that is directed against an antigen expressed by target cells and promotes cytotoxic activity against the target cells.

In several embodiments, the tumor target is selected from a ligand for the NKG2D receptor, CD19, CD70, BCMA, or CD38. In several embodiments, the tumor target is a ligand for the NKG2D receptor. In several embodiments, the tumor target is CD19. In several embodiments, the tumor target is CD70. In several embodiments, the tumor target is BCMA. In several embodiments, the tumor target is CD38. In several embodiments, the antigen is selected from a ligand for the NKG2D receptor, CD19, CD70, BCMA, or CD38. In several embodiments, the antigen is a ligand for the NKG2D receptor. In several embodiments, the antigen is CD19. In several embodiments, the antigen is CD70. In several embodiments, the antigen is BCMA. In several embodiments, the antigen is CD38.

In several embodiments, the CAR comprises a transmembrane domain. In several embodiments, the CAR comprises an intracellular signaling region. In several embodiments, the CAR comprises a transmembrane domain and an intracellular signaling region. In several embodiments, the transmembrane domain comprises CD8, CD28, or a portion thereof. In several embodiments, the transmembrane domain comprises CD8 or a portion thereof. In several embodiments, the transmembrane domain comprises CD28 or a portion thereof. In several embodiments, the transmembrane domain comprises CD8a. In several embodiments, the intracellular signaling region comprises a CD3zeta domain. In several embodiments, the intracellular signaling region comprises an intracellular signaling domain of an OX40, 4-1BB, CD28, or a signaling portion thereof. In several embodiments, the intracellular signaling region comprises an intracellular signaling domain of OX40. In several embodiments, the intracellular signaling region comprises an intracellular signaling domain of 4-1BB. In several embodiments, the intracellular signaling region comprises an intracellular signaling domain of CD28.

In several embodiments, the method comprises engineering NK cells of the expanded NK cell population, the genetically edited NK cell population, or the engineered NK cell population to express membrane-bound interleukin 15 (mbIL15). In several embodiments, the method comprises engineering NK cells of the expanded NK cell population to express mbIL15. In several embodiments, the method comprises engineering NK cells of the genetically edited NK cell population to express mbIL15. In several embodiments, the method comprises engineering NK cells of the engineered NK cell population to express mbIL15. In several embodiments, NK cells of the expanded NK cell population, the genetically edited NK cell population, or the engineered NK cell population are engineered to express membrane-bound interleukin 15 (mbIL15). In several embodiments, NK cells of the expanded NK cell population are engineered to express mbIL15. In several embodiments, NK cells of the genetically edited NK cell population are engineered to express mbIL15. In several embodiments, NK cells of the engineered NK cell population are engineered to express mbIL15.

In several embodiments, the engineering of the NK cells is concurrent with or after the genetic editing. In several embodiments, the engineering of the NK cells is concurrent with the genetic editing. In several embodiments, the engineering of the NK cells is after the genetic editing.

In several embodiments, a population of NK cells produced by any of the methods described herein exhibits increased natural cytotoxicity against target cells (e.g., not CAR-mediated cytotoxicity), as compared to a population of NK cells produced by a method not comprising selecting a biological sample having a threshold percentage of cells expressing an iKIR.

In several embodiments, the immunotherapy is for treating a disease or condition in a subject. In some embodiments, the disease or condition is an autoimmune disease or cancer. In several embodiments, the immunotherapy is for treating an autoimmune disease in a subject. In In several embodiments, the immunotherapy is for treating a cancer in a subject. In several embodiments, the cancer is a hematologic malignancy. In several embodiments, the cancer is a leukemia or a lymphoma. In several embodiments, the CAR binds to an antigen expressed by the cancer. In several embodiments, the CAR binds to an antigen not expressed, or no longer expressed, by cells of the cancer. In several embodiments, the NK cells of the biological sample are allogeneic to the subject.

### BRIEF DESCRIPTION OF THE DRAWINGS

The descriptions of the figures below are related to experiments and results that represent non-limiting embodiments of the inventions disclosed herein.

FIGS. 1A-1C relate to KIR expression on donor cells (1A, modified from Ewen et al Eur. J. Immunol. 2018. 48:355-365) and the effects of the presence or absence of stimulatory interleukins on expansion (1B) and cytotoxicity (1C) of NK cells from various donors, when untransduced, or when engineered to express an anti-CD19 chimeric antigen receptor (CAR).

FIGS. 2A-2F depict data related to trends of the cytotoxic potency and characterization of donor NK cells in view of the donor KIR haplotype and without culture supplementation with stimulatory interleukins (2A) or in the presence of stimulatory interleukins (2B).

FIG. 2C shows a summary of DNA-based high-resolution genotypic analysis of HLA & KIR performed on the 12 NK donors used to generate the data in 2A and 2B as well as KIR B content group determined using the IPD-KIR database. FIG. 2D shows data related to marker expression on NK cells expanded using engineered feeder cells with or without soluble IL12/IL18 cytokines and genetically modified with a retroviral CD19-CAR-mbIL-15 construct. Cells were characterized by flow cytometry on Days 0 & 14. FIG. 2E shows a volcano plot with changes of various markers in NK cells when expanded in the presence or the absence of IL12 and IL18. FIG. 2F shows gene expression data (with or without IL12 and IL18) and an upregulation of genes associated with activation of NK cells. FIGS. 2G-2J show cytotoxicity data for NK cells based on their KIR haplotype on different tumor cell lines.

FIGS. 3A-3B depict data related to correlation of donor NK cell potency and KIR haplotype and the impact of CMV status of the donor.

FIGS. 4A-4B show data related to the correlation of culture supplementation with stimulating interleukins and activating KIR haplotype.

FIGS. 5A-5B show data related to the correlation of culture supplementation with stimulating interleukins and inhibitory KIR haplotype.

FIGS. 6A-6B show data related to the correlation of donor cell expansion with cytotoxicity.

FIGS. 7A-7B show data related to the enhancement of cytotoxicity at an effector-to-target (E:T) ratio of 1:4 based on culture media supplementation with stimulating interleukins.

FIGS. 8A-8B show additional data related to the discrimination between cytotoxicity of selected donor NK cells at an E:T ratio of 1:8.

FIGS. 9A-9F show cytotoxicity data of expanded NK cells at day 21 of growth.

FIGS. 9A-9B show cytotoxicity curves for NK cells against tumor cells with and without stimulating interleukins. FIGS. 9C-9F show data related to the expression of various analytes in the supernatants from NALM-6 tumor cytotoxicity assays. FIG. 9C shows levels of IFN-g, FIG. 9D shows levels of GM-CSF, FIG. 9E shows levels of MIP-1a, and FIG. 9F shows levels of Perforin.

FIG. 10 shows data related to cytotoxicity quantification over 144 hours.

FIG. 11 shows data related to cytotoxicity quantification over 24 hours.

FIGS. 12A-12B show data related to the use of varied E:T ratios in order to discriminate among higher cytotoxicity donor cells.

FIG. 13 shows a schematic of an expansion protocol according to embodiments disclosed herein.

FIGS. 14A-14D show data related to expansion of cells using an embodiment of the expansion processes disclosed herein. 14A shows data from a first replicate of the experiment and 14B shows data from a replicate of the expansion from the day 56 time point (prior time points are same data as in 14A). FIGS. 14C and 14D tabulate the data of FIGS. 14A and 14B, respectively.

FIGS. 15A-15D show data related to fold expansion of cells using an embodiment of the expansion processes disclosed herein. 15A shows data from a first replicate of the experiment and 15B shows data from a replicate of the expansion from the fifth pulse (prior time points are same data as in 15A). FIGS. 15C and 15D tabulate the data of FIGS. 15A and 15B, respectively.

FIGS. 16A-16C show data summarizing the expansion of cells according to embodiments disclosed herein during the final 14 days of an expansion. FIG. 16A shows a first replicate of the expansion experiment, and FIG. 16B shows an additional replicate with the presence (solid) or absence (open) of IL12/18 at the inception of this final culture period. FIG. 16C tabulates the data of FIG. 16B.

FIGS. 17A-17B relate to CAR expression data. FIG. 17A tabulates data related to the percent of cells expressing the non-limiting CAR transduced into the cells earlier in the expansion process. FIG. 17B shows similar data for the percentage of cells expressing the CD19 CAR and mbIL15 at various stages of the expansion process as disclosed herein versus a pre-existing expansion approach (SP—standard process, also referred to as NKSTIM). MCB—Master Cell Bank; WCB—Working Cell Bank; FP—Final Product.

FIGS. 18A-18D summarize cytotoxicity of cell from various donors at the indicated point in the expansion process.

FIGS. 19A-19D show data related to the trends in expression of various markers during the expansion process. FIGS. 19A-19D show the trends of the expression of the listed markers with pulse number in NK cells from three donors. FIG. 19D shows expression of NKG2D (a non-limiting example of an activating receptor) in NK cells from three donors at various stages of the production methods disclosed herein, versus pre-expansion and Standard Process.

FIGS. 20A-20H show data related to expression of various markers by expanded cells. FIG. 20A-20D show data related to expression of the indicated markers on NK cells expanded according to embodiments disclosed herein with, or without, IL12 and IL18. FIGS. 20E-20F show data related to the expression of eomesodermin (Eomes) by NK cells expanded according to embodiments disclosed herein with, or without, IL12 and IL18. FIG. 20G shows expression of various markers of NK cell exhaustion by NK cells from a donor. FIG. 20H summarizes expressing of TIGIT across three donors at the WCB phase of expansion.

FIGS. 21A-21D show data related to expression of p16 by cells during the indicated points of an expansion.

FIGS. 22A-22F show data related to chromosomal stability and cytotoxicity of NK cells expanded according to methods disclosed herein. FIGS. 22A-22B show results of a chromosomal analysis of pre- and post-expansion NK cells. No chromosomal aberrations were observed after expansion. FIGS. 22C-22F show data related to the maintained cytotoxicity against tumor cell lines or non-tumor cell lines expressing CD19 by NK cells expanded according to embodiments disclosed herein.

FIGS. 23A-23E show data related to the expansion of NK cells from cord blood or peripheral blood using expansion methods as provided for herein. FIG. 23A shows data related to expansion of untransduced NK cells (either from cord or peripheral blood) over 14 days. FIG. 23B shows data related to the expansion of NK cells (either from cord or peripheral blood) engineered to express an anti-CD19 CAR over 14 days. FIG. 23C shows data related to expansion of NK cells (either from cord or peripheral blood) engineered to express an anti-CD19 CAR over 70 days. FIG. 23D provides a summary of the fold expansion of NK cells for each phase of the expansion methods provided for herein. FIG. 23E shows data related to the degree of expansion with each reintroduction (e.g., “pulse”) of feeder cells.

FIGS. 24A-24M show data related to expression of various markers by the NK cells (either from cord or peripheral blood) during expansion. FIG. 24A shows expression of NKG2C. FIG. 24B shows expression of CD39. FIG. 24C shows expression of TIM3. FIG. 24D shows expression of OX40L. FIG. 24E shows expression of CD62L. FIG. 24F shows expression of LAG3. FIG. 24G shows expression of PD1. FIG. 24H shows expression of CD56. FIG. 24I shows expression of CD16. FIG. 24J shows expression of NKG2A. FIG. 24K shows expression of ILT2. FIG. 24L shows expression of CD57. FIG. 24M shows expression of TIGIT.

FIGS. 25A-25M show data related to expression of various additional markers by the NK cells (either from cord or peripheral blood) during expansion. FIG. 25A shows expression of KIR2DL2/L3. FIG. 25B shows expression of KIR2DS4. FIG. 25C shows expression of KIR2DL1/DS5. FIG. 25D shows expression of KIR3DS1. FIG. 25E shows expression of KIR2DL2/L3/S2. FIG. 25F shows expression of LAIR1. FIG. 25G shows expression of CD27. FIG. 25H shows expression of CD56. FIG. 25I shows expression of CD16.

FIG. 25J shows expression of NKG2A. FIG. 25K shows expression of KIR3DL1. FIG. 25L shows expression of KLRG1. FIG. 25M shows expression of CD160.

FIGS. 26A-26M show data related to expression of various additional markers by the NK cells (either from cord or peripheral blood) during expansion. FIG. 26A shows expression of NKp30. FIG. 26B shows expression of 41BB. FIG. 26C shows expression of NKp80. FIG. 26D shows expression of NKp44. FIG. 26E shows expression of CD25. FIG. 26F shows expression of NKp46. FIG. 26G shows expression of DNAM1. FIG. 26H shows expression of CD56. FIG. 26I shows expression of CD16. FIG. 26J shows expression of 2B4. FIG. 26K shows expression of GITR. FIG. 26L shows expression of NKG2D. FIG. 26M shows expression of CD69.

FIGS. 27A-27B show data related to expression of various markers by NK cells during the expansion process. FIG. 27A shows the expression of CD57 and the NKG2C receptor during expansion of peripheral blood (PB) NK cells or cord blood (CB) NK cells. FIG. 27B shows the expression of KIRs and the NKG2A receptor during expansion of PB NK cells or CB NK cells.

FIGS. 28A-28I show data related to CD19 CAR expression on the PB NK or CB NK cells at 14 versus 70 days of expansion. FIG. 28A shows CD19 CAR expression on CB NK cells from a first donor expanded for 14 days. FIG. 28B shows CD19 CAR expression on CB NK cells from a second donor expanded for 14 days. FIG. 28C shows CD19 CAR expression on CB NK cells from a third donor expanded for 14 days. FIG. 28D shows CD19 CAR expression on CB NK cells from a fourth donor expanded for 14 days. FIG. 28E shows CD19 CAR expression on PB NK cells from a first donor expanded for 14 days. FIG. 28F shows CD19 CAR expression on CB NK cells from the third CB donor expanded for 70 days. FIG. 28G shows CD19 CAR expression on CB NK cells from the fourth CB donor expanded for 70 days. FIG. 28H shows CD19 CAR expression on PB NK cells from the first CB donor expanded for 70 days. FIG. 28I provides summary data of CD19 CAR expression for each donor at 14 (D14) or 70 (D70) days of expansion.

FIGS. 29A-29C show data related to cytotoxicity for expanded NK cells. FIG. 29A shows cytotoxicity data of CB or PB NK cells expressing a CD19-directed CAR against Raji cells (Burkitt lymphoma, after 71 hours of co-culture) at the indicated E:T ratios. FIG. 29B shows cytotoxicity data of CB or PB NK cells expressing a CD19-directed CAR against NALM6 cells (B cell precursor leukemia, after 71 hours of co-culture) at the indicated E:T ratios. FIG. 29C shows cytotoxicity data of CB or PB NK cells expressing a CD19-directed CAR against HT-29-CD19 cells (colorectal adenocarcinoma engineered to ectopically express CD19, after 47 hours of co-culture) at the indicated E:T ratios.

FIGS. 30A-30C show data related to the cytotoxicity of NK cells (either CB NK or PB NK cells) after either 14 or 70 days of expansion according to methods provided for herein. FIG. 30A shows cytotoxicity data of CB or PB NK cells expanded for 14 or 70 days and expressing a CD19-directed CAR against Raji cells (Burkitt lymphoma, after 72 hours of co-culture) at the indicated E:T ratios. FIG. 30B shows cytotoxicity data of CB or PB NK cells expanded for 14 or 70 days and expressing a CD19-directed CAR against NALM6 cells (B cell precursor leukemia, after 72 hours of co-culture) at the indicated E:T ratios. FIG. 30C shows cytotoxicity data of CB or PB NK cells expanded for 14 or 70 days and expressing a CD19-directed CAR against HT-29-CD19 cells (colorectal adenocarcinoma engineered to ectopically express CD19, after 48 hours of co-culture) at the indicated E:T ratios.

FIG. 31 shows the genotype of a peripheral blood NK (PBNK) cell donor for exemplary inhibitory receptors and ligands.

FIG. 32A shows pre-(left panels) and post-expansion (right panels) flow cytometric analysis of unsorted PB NK cells (top panel) or PB NK cells sorted pre-expansion on NKG2A and KIR2DL3 expression. FIG. 32B shows the quantification of fold-expansion of the unsorted and sorted PB NK cells from FIG. 32A.

FIG. 33A shows the cytotoxicity of unsorted and sorted CD19 CAR-transduced PB NK cells following co-culture with CD19-expressing NALM6 cells at the indicated E:T ratios.

FIG. 33B shows the cytotoxicity of sorted untransduced PB NK cells following co-culture with HL-60 cells not expressing CD19 at the indicated E:T ratios.

### DETAILED DESCRIPTION

While cancer immunotherapy, or cellular therapy for other diseases, has advanced greatly in terms of the ability to engineer cells to express constructs of interest, there is still a need for clinically relevant number of those cells for patient administration. This is particularly important when the underlying native immune cell to be engineered and later administered is less prevalent than other immune cell types. This requires either starting with a larger amount of starting material, which may not be practical, or developing more efficient methods and compositions to expand (in some cases preferentially) the immune cell of interest, such as an NK cell. There are therefore provided herein, in several embodiments, methods for screening donors of immune cells for those who may exhibit a particular predisposition to enhanced expansion and/or enhanced cytotoxicity and compositions and methods that advantageously allow for the unexpectedly robust expansion of NK cells. There are also provided herein methods for enriching for NK cells that may exhibit a particular predisposition to enhanced expansion and/or enhanced cytotoxicity.

In several embodiments, there are provided populations of expanded and activated NK cells derived from co-culturing a modified “feeder” cell disclosed herein with a starting population of immune cells and supplementing the co-culture with various cytokines at certain time points during the expansion.

### I. NK Cell Enrichment

Existing methods for generating engineered immune cells, such as NK cells engineered to express an extracellular receptor, may include steps where populations of NK cells expand (e.g., in culture). The ability to achieve consistent and sufficient expansion of NK cells is advantageous in the context of engineering NK cells for downstream uses, such as immunotherapy, where relatively large numbers of engineered immune cells are often needed. Large numbers of cells are particularly required for NK cell therapies, as opposed to e.g., T cell therapies, as NK cells are known to exhibit limited in vivo expansion and persistence. In some cases, however, a portion of an immune (e.g., NK) cell population may not exhibit, or may not exhibit sufficient, expansion to meet this need.

The methods and cell populations provided herein address this issue. The provided methods and immune cell populations (e.g., genetically edited and/or engineered NK cell populations) relate to enriching for populations of educated NK cells that exhibit improved or more rapid expansion. NK cells become “educated” by interaction of inhibitory KIRs (iKIRs) with MHC ligands. Educated NK cells subsequently express one or more iKIRs on their surface. As described herein, it was observed that educated NK cells expressing an inhibitory KIR (iKIR+) exhibited substantial expansion during culture, such that enriching for such cells (e.g., from a donor cell sample) could yield increased quantities of NK cells in expanded populations. It is contemplated herein that enrichment for educated NK cells can be achieved by selecting for cells based on expression (e.g., surface expression) of an iKIR selected from KIR2DL1, KIR2DL2, KIR2DL3, KIR3DL1, KIR3DL2, KIR3DL3, KIR2DL5, or any combination thereof. Such enrichment for educated NK cells may increase the consistency and robustness of engineered cell manufacturing methods, both within and among donors, such as compared to a process in which iKIR+ NK cells are not enriched.

In some embodiments, a biological sample from a donor is enriched for NK cells expressing an iKIR. In some embodiments, the biological sample is enriched for cells expressing one, two, three, four, five, six, or seven iKIRs. In some embodiments, the iKIR comprises KIR2DL1. In some embodiments, the iKIR comprises KIR2DL1. In some embodiments, the iKIR comprises KIR2DL2. In some embodiments, the iKIR comprises KIR2DL3. In some embodiments, the iKIR comprises KIR3DL1. In some embodiments, the iKIR comprises KIR3DL2. In some embodiments, the iKIR comprises KIR3DL3. In some embodiments, the iKIR comprises KIR2DL5. In some embodiments, the iKIR is KIR2DL1. In some embodiments, the iKIR is KIR2DL1. In some embodiments, the iKIR is KIR2DL2. In some embodiments, the iKIR is KIR2DL3. In some embodiments, the iKIR is KIR3DL1. In some embodiments, the iKIR is KIR3DL2. In some embodiments, the iKIR is KIR3DL3. In some embodiments, the iKIR is KIR2DL5.

In some embodiments, enrichment for cells expressing an iKIR from the biological sample comprises positive selection for cells expressing an iKIR. In some embodiments, enrichment for cells expressing an iKIR from the biological sample comprises negative selection for cells not expressing an iKIR from the biological sample.

Observations herein further demonstrate that cells expressing an iKIR and not expressing NKG2A, a HLA class I specific inhibitory receptor, may exhibit further improved expansion. Thus, in some embodiments, it is contemplated that enrichment for cells expressing an iKIR and not expressing NKG2A (iKIR+NKG2A−) will enrich for cells with a greater proliferative capacity, such as compared to cells expressing NKG2A. In some embodiments, the methods comprise enriching for cells not expressing NKG2A from a biological sample.

In some embodiments, enrichment for cells not expressing NKG2A from the biological sample comprises positive selection for cells not expressing NKG2A. In some embodiments, enrichment for cells not expressing NKG2A from the biological sample comprises negative selection for cells expressing NKG2A from the biological sample.

It will be appreciated that selection for expression of iKIR+ and NKG2A− can be carried out sequentially (in either order) or simultaneously. For example, selection of iKIR+NKG2A− cells can be carried out sequentially. In some embodiments, a first selection for iKIR+ cells can be carried out, and iKIR+ cells from the first selection can be subjected to a second selection for NKG2A− cells. In other embodiments, a first selection for NKG2A− cells can be carried out, and NKG2A− cells from the first selection can be subjected to a second selection for iKIR+ cells. Alternatively, selection of iKIR+NKG2A− cells can be carried out simultaneously. As a non-limiting example, a single donor cell population could be simultaneously incubated with an anti-iKIR antibody and an anti-NKG2A antibody, where cells bound only to the anti-iKIR antibody are retained (e.g., selected), and cells bound only to the anti-NKG2A antibody or bound to both of the anti-iKIR and anti-NKG2A antibodies are discarded.

In some of any of the provided embodiments, the antibody is immobilized on a solid surface. In some of any of the provided embodiments, the solid surface is a magnetic particle.

In some aspects, a sample or population of cells to be enriched is incubated with a selection reagent containing small, magnetizable or magnetically responsive material, such as magnetically responsive particles or microparticles, such as paramagnetic beads (e.g., such as Dynalbeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select. In some aspects, the selection reagent is or includes a paramagnetic bead and an attached antibody or antigen binding fragment thereof that binds to or recognizes an iKIR or NKG2A. The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample or cell population.

In some aspects, selection is achieved in a procedure in which the sample or cell population is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some of any of the provided embodiments, the antibody is immobilized on or attached to an affinity chromatography matrix. In some aspects, cells of a sample or cell population, e.g. NK cells, are selected by chromatographic isolation, including by column chromatography (e.g., affinity chromatography). In some embodiments, cells, e.g. iKIR+ NK cells or iKIR+NKG2A− NK cells, are selected or enriched by chromatographic isolation, including affinity chromatography. In some embodiments, cells are selected by positive selection using column chromatography, such as by eluting and collecting cells that bind to the chromatography matrix. In some embodiments, cells are selected by negative selection using column chromatography, such as by collecting cells that do not bind to the chromatography matrix.

Provided herein are enriched cell populations comprising NK cells produced by any of the methods as described herein.

In some embodiments, the enriched cell population is enriched for iKIR+ NK cells. In some embodiments, an enriched cell population comprises at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% iKIR+ NK cells. In some embodiments, an enriched cell population comprises at least about 20% iKIR+ NK cells. In some embodiments, an enriched cell population comprises at least about 30% iKIR+ NK cells. In some embodiments, an enriched cell population comprises at least about 40% iKIR+ NK cells. In some embodiments, an enriched cell population comprises at least about 50% iKIR+ NK cells. In some embodiments, an enriched cell population comprises at least about 60% iKIR+ NK cells. In some embodiments, an enriched cell population comprises at least about 70% iKIR+ NK cells. In some embodiments, an enriched cell population comprises at least about 80% iKIR+ NK cells. In some embodiments, an enriched cell population comprises at least about 90% iKIR+ NK cells. In some embodiments, an enriched cell population comprises at least about 95% iKIR+ NK cells. In some embodiments, an enriched cell population comprises at least about 99% iKIR+ NK cells. In some embodiments, the enriched cell population is substantially devoid of iKIR− NK cells.

In some embodiments, the enriched cell population is enriched for NKG2A− NK cells. In some embodiments, an enriched cell population comprises at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% NKG2A− NK cells. In some embodiments, an enriched cell population comprises at least about 20% NKG2A− NK cells. In some embodiments, an enriched cell population comprises at least about 30% NKG2A− NK cells. In some embodiments, an enriched cell population comprises at least about 40% NKG2A− NK cells. In some embodiments, an enriched cell population comprises at least about 50% NKG2A− NK cells. In some embodiments, an enriched cell population comprises at least about 60% NKG2A− NK cells. In some embodiments, an enriched cell population comprises at least about 70% NKG2A− NK cells. In some embodiments, an enriched cell population comprises at least about 80% NKG2A− NK cells. In some embodiments, an enriched cell population comprises at least about 90% NKG2A− NK cells. In some embodiments, an enriched cell population comprises at least about 95% NKG2A− NK cells. In some embodiments, an enriched cell population comprises at least about 99% NKG2A− NK cells. In some embodiments, the enriched cell population is substantially devoid of NKG2A+ NK cells.

In some embodiments, the enriched cell population is enriched for iKIR+NKG2A− NK cells. In some embodiments, an enriched cell population comprises at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% iKIR+NKG2A− NK cells. In some embodiments, an enriched cell population comprises at least about 20% iKIR+NKG2A− NK cells. In some embodiments, an enriched cell population comprises at least about 20% iKIR+NKG2A− NK cells. In some embodiments, an enriched cell population comprises at least about 30% iKIR+NKG2A− NK cells. In some embodiments, an enriched cell population comprises at least about 40% iKIR+NKG2A− NK cells. In some embodiments, an enriched cell population comprises at least about 50% iKIR+NKG2A− NK cells. In some embodiments, an enriched cell population comprises at least about 60% iKIR+NKG2A− NK cells. In some embodiments, an enriched cell population comprises at least about 70% iKIR+NKG2A− NK cells. In some embodiments, an enriched cell population comprises at least about 80% iKIR+NKG2A− NK cells. In some embodiments, an enriched cell population comprises at least about 90% iKIR+NKG2A− NK cells. In some embodiments, an enriched cell population comprises at least about 95% iKIR+NKG2A− NK cells. In some embodiments, an enriched cell population comprises at least about 99% iKIR+NKG2A− NK cells. In some embodiments, the enriched cell population is substantially devoid of iKIR−NKG2A+ NK cells.

In some embodiments, an enriched cell population is subjected to one or more downstream manufacturing steps, such as genetic editing and/or engineering, such as described herein. In some embodiments, an enriched cell population is genetically edited. In some embodiments, an enriched cell population is genetically engineered (e.g., to express a chimeric receptor). In some embodiments, an enriched cell population is genetically edited and genetically engineered.

### II. Donor Selection—Characteristics and Methods of Selecting Donors

As many types of cancer immunotherapy rely on donor-derived cells, in particular in the allogeneic context, the selection of a donor can be a key component of generation of a successful therapeutic regimen. As discussed in more detail herein, in several embodiments, allogeneic donors are used, for example in the development of off the shelf cancer immunotherapies, in particular those one or more types of immune cells, such as Natural Killer (NK) cells.

Various donor characteristics can be evaluated, alone or in combination, in order to improve one or more aspects of the donor-derived cells. For example, in several embodiments, an optimal donor would exhibit one or more of (i) predisposed to expansion in culture, (ii) readily transduced (e.g., with a vector for delivery of a chimeric antigen receptor (CAR) or other payload (gene editing machinery), and (iii) potent baseline cytotoxicity. One (or combinations) of these, or other, characteristics discussed herein may be a weighted factor in making a given donor an optimal candidate from which to develop a master cell bank (MCB) and/or a working cell bank (WCB) such that a single donor can yield numerous identical doses of cells for use in allogeneic cell therapy.

As will be discussed in more detail below, and in the examples, multiple approaches can be used to evaluate and screen potential donors. For example, in several embodiments, protein expression techniques, such as flow cytometry to measure certain cell surface markers is used. In several embodiments, various assays are used to measure the cytokine secretome of a cell, or to determine its chemokine/granule release potential. In several embodiments, gene expression is evaluated to determine what potential genes that could impact or hinder cell expansion are expressed. In several embodiments, cells from a potential donor are genotyped, for example with respect to their HLA profile or Killer Cell Ig-like Receptors (KIR) profile. In several embodiments, the memory-like characteristics (e.g., memory or memory-like NK cell characteristics) are evaluated (e.g., cytomegalovirus positivity of donor, NKG2C expression, and/or ability for clonal expansion). In several embodiments, combinations of such methods are used. For example, and as described herein, in some embodiments, the Killer Cell Ig-like Receptors (KIR) profile of a potential donor is evaluated in combination with NKG2A expression. In some embodiments the inhibitory KIR profile of a donor is evaluated in combination with NKG2A expression. In several embodiments, such methods can be used for correlating one or more of the characteristics assessed with potency and/or ability for expansion.

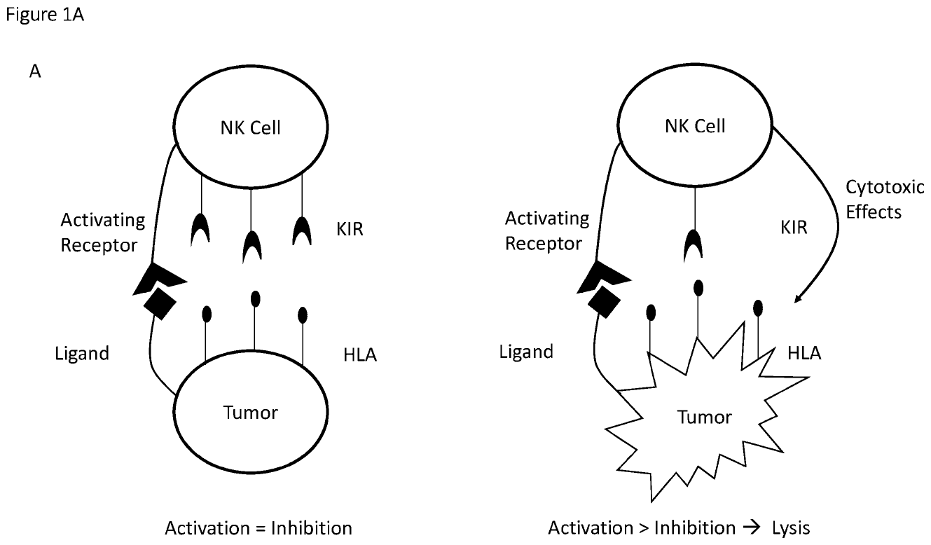

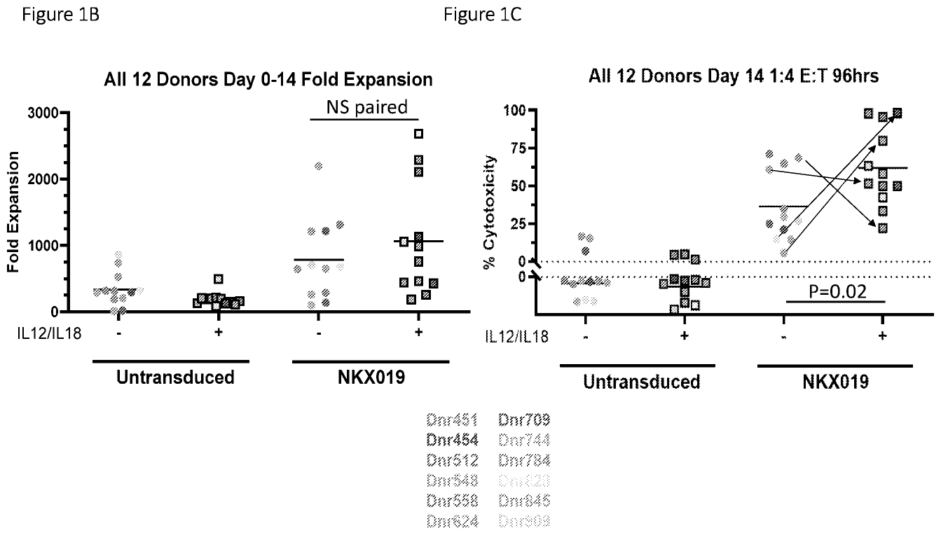

In several embodiments, as disclosed herein, NK cells are collected from a donor, engineered and/or edited and expanded in culture for use in cellular therapy. NK cell functions are regulated by a diversity of activating and inhibitory cell surface receptors. As mentioned above, one of these cell surface receptor families controlling the effector function of NK cells are the KIRs. Six of them are activating KIRs (aKIR), including KIR2DS1, KIR2DS2, KIR2DS3, KIR2DS4, KIR2DS15, and KIR3DS1. In contrast, seven are inhibitory KIRs (iKIR), including KIR2DL1, KIR2DL2, KIR2DL3, KIR3DL1, KIR3DL2, KIR3DL3, and KIR2DL5. KIR2DL4 exhibits both activating and inhibitory properties. Finally, two are believed to be pseudogenes (KIR2DP1 and KIR3DP1). In mature NK cells, iKIR inhibit cytotoxicity if bound to HLA (and other) tumor ligands while aKIR increase cytotoxicity if bound to HLA (and other) tumor ligands (see FIG. 1A, modified from Ewen et al Eur. J. Immunol. 2018. 48:355-365). KIRs may either inhibit or stimulate NK cell activity after engagement with specific human leukocyte antigen (HLA) class I ligands and, despite their high genetic variability and particularly diverse KIR/HLA ligand interactions, the KIRs allow the NK cells to self-discriminate healthy cells from transformed or pathogen-infected cells and regulate their effector function. Thus, according to several embodiments, the NK cells (or other immune cells collected from a potential donor) are evaluated with respect to their KIR profile, including in one embodiment assessing aKIR expression, in one embodiment assessing iKIR expression, and in several embodiments, assessing both aKIR and iKIR expression and calculating a ratio that is predictive of the future expandability and/or cytotoxicity of the cells. In some embodiments, immune cells (e.g., NK cells) are evaluated with respect to their iKIR profile, including in one embodiment assessing iKIR expression (e.g., assessing expression of at least one of KIR2DL1, KIR2DL2, KIR2DL3, KIR3DL1, KIR3DL2, KIR3DL3, and KIR2DL5). In some embodiments, immune cells (e.g., NK cells are) are evaluated with respect to their expression of NKG2A. In some embodiments, iKIR and NKG2A expression is evaluated in immune cells (e.g., NK cells).

As discussed in more detail below in the examples, according to several embodiments, donor potency (e.g., eventual cytotoxicity) can be driven by KIR-based or non-KIR-based factors. KIR drives potency via two different mechanisms, according to some embodiments. In several embodiments, there is a mismatch of donor (and thus therapeutic) cell iKIR expression with a patient tumor HLA. This mismatch means that the patient's tumor cells do not engage the iKIR (and thus less or no NK cell inhibition results) and therefore the tumor cells are more readily killed. Alternatively, or in addition to, the above, those donors who are KIR Haplotype Group B exhibit higher frequencies of activating KIR are thus more potent, according to several embodiments. Non-KIR-based potency, according to some embodiments, exhibit a robust response to stimulatory molecules (such as IL12 and/or IL18) that are used in certain embodiments of immune cell expansion, which imparts to them enhanced cytotoxicity. In some embodiments, a donor is preferred because their cells exhibit both KIR and non-KIR-based potency increases (e.g., after expansion).

In several embodiments, a candidate donor is identified and a blood sample comprising immune cells (e.g, NK cells) is obtained from the candidate donor. In several embodiments, the sample is divided into multiple portions, with one or more being subjected to a screening process, and the others being saved and subsequently used as donor cells for expansion or discarded. In several embodiments, the immune cells are separated into at least in part, substantially or completely isolated NK cells. In several embodiments, the expression of at least one of KIR2DS1, KIR2DS2, KIR2DS3, KIR2DS4, KIR2DS15, and KIR3DS1 (e.g., by the NK cells) is evaluated. In several embodiments, the expression of at least one of KIR2DL1, KIR2DL2, KIR2DL3, KIR3DL1, KIR3DL2, KIR3DL3, and KIR2DL5 is evaluated. In several embodiments, the expression of at least one of KIR2DS1, KIR2DS2, KIR2DS3, KIR2DS4, KIR2DS15, and KIR3DS1 is evaluated and also the expression of at least one of KIR2DL1, KIR2DL2, KIR2DL3, KIR3DL1, KIR3DL2, KIR3DL3, and KIR2DL5 is evaluated. In several embodiments, wherein expression of both at least one aKIR and at least one iKIR is evaluated, a comparison of the amount of aKIR to the amount of iKIR is made. In several embodiments, a raw expression signal comparison is used (e.g., signal intensities). In several embodiments, normalizations of expression are performed, e.g., to a housekeeping gene/protein. In several embodiments, a ratio of aKIR to iKIR expression is calculated. In several embodiments, the ratio is predictive of the future potency of the cells, as it represents the probability that an NK cell will generate greater activating KIR function versus inhibitory KIR function. In several embodiments, a candidate donor with an aKIR:iKIR ratio of at least about 3:1, about 3.5:1, about 4:1, about 4.5:1, about 5:1, about 5.5:1, about 6:1, about 6.5:1, about 7:1, about 7.5:1, about 8:1, about 8.5:1 or greater (and including any ratio between those listed) is determined to be a preferred donor (a donor whose cells are later engineered/edited and/or expanded). In several embodiments, a preferred donor has an aKIR:iKIR ratio of about 3:1, 5:1, 8:1, 10:1, 12:1, 15:1, 18:1, 20:1 or greater (including any ratio between those listed). In several embodiments, a donor can be selected based on the number of aKIRs that are expressed. In several embodiments, a candidate donor can be determined to be a preferred donor based on the donor's cells expressing at least 2, at least 3, or at least 4 aKIRs. In several embodiments, a preferred donor population of cells will express fewer than a full contingent of iKIRs, for example less than 5, less than 4, less than 3 or less than 2 iKIRs.

In several embodiments, the expression of at least one iKIR (e.g., at least one of KIR2DL1, KIR2DL2, KIR2DL3, KIR3DL1, KIR3DL2, KIR3DL3, and KIR2DL5) is evaluated. In several embodiments, the expression of at least one iKIR (e.g., at least one of KIR2DL1, KIR2DL2, KIR2DL3, KIR3DL1, KIR3DL2, KIR3DL3, and KIR2DL5) is evaluated in NK cells from a candidate donor. In some embodiments, the expression of KIR2DL1 is evaluated in NK cells from a candidate donor. In some embodiments, the expression of KIR2DL2 is evaluated in NK cells from a candidate donor. In some embodiments, the expression of KIR2DL3 is evaluated in NK cells from a candidate donor. In some embodiments, the expression of KIR3DL1 is evaluated in NK cells from a candidate donor. In some embodiments, the expression of KIR3DL2 is evaluated in NK cells from a candidate donor. In some embodiments, the expression of KIR3DL3 is evaluated. In some embodiments, the expression of KIR2DL5 is evaluated in NK cells from a candidate donor. In several embodiments, the expression of NKG2A is evaluated. In several embodiments, the expression of NKG2A is evaluated in NK cells from a candidate donor. In several embodiments, the expression of at least one iKIR, and the expression of NKG2A, is evaluated. In several embodiments, the expression of at least one iKIR, and the expression of NKG2A, is evaluated in NK cells from a candidate donor. In several embodiments, if cells (e.g., NK cells) from a donor sample express at least one iKIR, the donor is determined to be a preferred donor. In several embodiments, if cells (e.g., NK cells) from a donor sample express at least one iKIR and do not express NKG2A, the donor is determined to be a preferred donor. In several embodiments, if NK cells from a candidate donor sample express at least one iKIR, the donor is identified as a preferred donor. In several embodiments, if cells (e.g., NK cells) from a candidate donor sample express at least one iKIR and do not express NKG2A, the donor is identified as a preferred donor. In some embodiments, if a candidate donor is identified as a preferred donor, the sample from the preferred donor is used to produce a cell composition. For example, in some embodiments, a sample from a preferred donor is subjected to activation, expansion, genetic engineering, and/or genetic editing, thereby producing a cell composition. In some embodiments, the cell composition is for use in treating a disease or condition.

In some embodiments, the at least one iKIR comprises KIR2DL1, In some embodiments, the at least one iKIR comprises KIR2DL2. In some embodiments, the at least one iKIR comprises KIR2DL3. In some embodiments, the at least one iKIR comprises KIR3DL1, In some embodiments, the at least one iKIR comprises KIR3DL2, In some embodiments, the at least one iKIR comprises KIR3DL3, In some embodiments, the at least one iKIR comprises KIR2DL5.

In some embodiments, the at least one iKIR is KIR2DL1, In some embodiments, the at least one iKIR is KIR2DL2. In some embodiments, the at least one iKIR is KIR2DL3. In some embodiments, the at least one iKIR is KIR3DL1, In some embodiments, the at least one iKIR is KIR3DL2, In some embodiments, the at least one iKIR is KIR3DL3, In some embodiments, the at least one iKIR is KIR2DL5.

### III. Cells for Use in Immune Cell Expansion

Some embodiments of the methods and compositions provided herein relate to collection of a cell such as an immune cell (e.g., an NK cell), for example from a donor, and expansion of all or a subset of the collected cells in culture. In addition, in several embodiments, the cells are engineered and/or gene edited for use in, for example, cancer immunotherapy. For example, an immune cell, such as a NK cell, may be engineered to include a chimeric receptor such as a CD19-directed chimeric receptor, or engineered to include a nucleic acid encoding said chimeric receptor as described herein. Additional embodiments relate to engineering a second set of cells to express another cytotoxic receptor complex, such as an NKG2D chimeric receptor complex as disclosed herein. Still additional embodiments relate to the further genetic manipulation of NK cells (e.g., donor NK cells) to reduce, disrupt, minimize and/or eliminate the ability of the donor NK cell to be alloreactive against recipient cells (graft versus host disease).

Traditional anti-cancer therapies relied on a surgical approach, radiation therapy, chemotherapy, or combinations of these methods. As research led to a greater understanding of some of the mechanisms of certain cancers, this knowledge was leveraged to develop targeted cancer therapies. Targeted therapy is a cancer treatment that employs certain drugs that target specific genes or proteins found in cancer cells or cells supporting cancer growth, (like blood vessel cells) to reduce or arrest cancer cell growth. More recently, genetic engineering has enabled approaches to be developed that harness certain aspects of the immune system to fight cancers. In some cases, a patient's own immune cells are modified to specifically eradicate that patient's type of cancer. Various types of immune cells can be used, such as T cells, Natural Killer (NK cells), or combinations thereof, as described in more detail below.

To facilitate cancer immunotherapies, there are provided for herein polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. Also provided herein are polynucleotides, polypeptides, and vectors that encode chimeric antigen receptors (CAR) that comprise a target binding moiety (e.g., an extracellular binder of a ligand, or a tumor marker-directed chimeric receptor, expressed by a cancer cell) and a cytotoxic signaling complex. For example, some embodiments include a polynucleotide, polypeptide, or vector that encodes, for example a chimeric antigen receptor directed against a tumor marker, for example, CD19, CD38, CD123, CD70, Her2, mesothelin, Claudin 6, BCMA, EGFR, among others, to facilitate targeting of an immune (e.g., NK) cell to a cancer and exerting cytotoxic effects on the cancer cell. Other embodiments include a polynucleotide, polypeptide, or vector that encodes, for example a chimeric antigen receptor directed against a marker, for example, CD19 or BCMA, to facilitate targeting of an immune (e.g., NK) cell to a cell that mediates an autoimmune disease (e.g., a B cell and/or a plasma cell). Anti-CD19 chimeric receptors are described in PCT Application No. PCT/US2020/020824, which is incorporated herein by reference in its entirety. Anti-BCMA chimeric receptors are described in PCT Application No. PCT/US2022/073567, which is incorporated herein by reference in its entirety.

Also provided are engineered immune cells (e.g., NK cells) expressing such CARs. There are also provided herein, in several embodiments, polynucleotides, polypeptides, and vectors that encode a construct comprising an extracellular domain comprising two or more subdomains, e.g., first CD19-targeting subdomain comprising a CD19 binding moiety as disclosed herein and a second subdomain comprising a C-type lectin-like receptor and a cytotoxic signaling complex. Also provided are engineered immune cells (e.g., NK cells) expressing such bi-specific constructs. Methods of treating a disease or condition are provided for herein. In some embodiments, the disease or condition is an autoimmune disease or condition. Thus, methods of treating an autoimmune disease are also provided for herein. In some embodiments, the disease or condition is a cancer. Methods of treating cancer and other uses of such cells for cancer immunotherapy are also provided for herein.

Non-limiting examples of CAR constructs for expression in cells provided for herein are provided in Table 1 below:

| Identifier | SEQ ID NO | Identifier | SEQ ID NO |
|---|---|---|---|
| NKG2D Chimeric Receptor with | 1 | NKG2D Chimeric Receptor | 85 (e.g., 41) |
| mbIL15 DNA | | Amino Acid | |
| NKG2D Chimeric Receptor Amino | 71 (e.g., 2) | DNA | 42 |
| Acid with mbIL15 | | NK77.71_CD70_DB04_D02 | |
| | | with mbIL15 | |
| DNA NK19H-No Flag-1 with | 3 | AA | 102 (e.g., 43) |
| mbIL15 | | NK77.71_CD70_DB04_D02 | |
| AA NK19H-No Flag-1 with mbIL15 | 72 (e.g., 4) | DNA | 44 |
| | | NK77.17_CD70_DB02_B06 | |
| | | with mbIL15 | |
| DNA NK19H-No Flag-2 with | 5 | AA | 103 (e.g., 45) |
| mbIL15 | | NK77.17_CD70_DB02_B06 | |
| AA NK19H-No Flag-2 with mbIL15 | 73 (e.g., 6) | DNA | 46 |
| | | NK77.58_CD70_DB03_H08 | |
| | | with mbIL15 | |
| DNA NK19H-No Flag-3 with | 7 | AA | 104 (e.g., 47) |
| mbIL15 | | NK77.58_CD70_DB03_H08 | |
| AA NK19H-No Flag-3 with mbIL15 | 74 (e.g., 8) | AA NK19H-No Flag-1 | 86 (e.g., 48) |
| DNA NK19H-No Flag-4 with | 9 | AA NK19H-No Flag-2 | 87 (e.g., 49) |
| mbIL15 | | | |
| AA NK19H-No Flag-4 with mbIL15 | 75 (e.g., 10) | AA NK19H-No Flag-3 | 88 (e.g., 50) |
| DNA NK19H-No Flag-5 with | 11 | AA NK19H-No Flag-4 | 89 (e.g., 51) |
| mbIL15 | | | |
| AA NK19H-No Flag-5 with mbIL15 | 76 (e.g., 12) | AA NK19H-No Flag-5 | 90 (e.g., 52) |
| DNA NK19H-No Flag-6 with | 13 | AA NK19H-No Flag-6 | 91 (e.g., 53) |
| mbIL15 | | | |
| AA NK19H-No Flag-6 with mbIL15 | 77 (e.g., 14) | AA NK19H-No Flag-7 | 92 (e.g.,. 54) |
| DNA NK19H-No Flag-7 with | 15 | AA NK19H-No Flag-8 | 93 (e.g., 55) |
| mbIL15 | | | |
| AA NK19H-No Flag-7 with mbIL15 | 78 (e.g., 16) | AA NK19H-No Flag-9 | 94 (e.g., 56) |
| DNA NK19H-No Flag-8 with | 17 | AA NK19H-No Flag-10 | 95 (e.g., 57) |
| mbIL15 | | | |
| AA NK19H-No Flag-8 with mbIL15 | 79 (e.g., 18) | AA NK19H-No Flag-11 | 96 (e.g., 58) |
| DNA NK19H-No Flag-9 with | 19 | AA NK19H-No Flag-12 | 97 (e.g., 59) |
| mbIL15 | | | |
| AA NK19H-No Flag-9 with mbIL15 | 80 (e.g., 20) | mbIL15 DNA | 60 |
| DNA NK19H-No Flag-10 with | 21 | mbIL15 AA | 98 (e.g., 61) |
| mbIL15 | | | |
| AA NK19H-No Flag-10 with | 81 (e.g., 22) | NK77.17_CD70_DB02_B06 | 62 |
| mbIL15 | | construct [+mbIL15] | |
| DNA NK19H-No Flag-11 with | 23 | NK77.58_CD70_DB03_H08 | 63 |
| mbIL15 | | construct [+mbIL15] | |
| AA NK19H-No Flag-11 with | 82 (e.g., 24) | NK77.71_CD70_DB04_D02 | 64 |
| mbIL15 | | construct [+mbIL15] | |
| DNA NK19H-No Flag-12 with | 25 | NK77.17_CD70_DB02_B06 | 99 (e.g., 65) |
| mbIL15 | | construct [no FLAG/ | |
| | | GS2; +mbIL15] | |
| AA NK19H-No Flag-12 with | 83 (e.g., 26) | NK77.58_CD70_DB03_H08 | 100 (e.g., 66) |
| mbIL15 | | construct [no FLAG/ | |
| | | GS2; +mbIL15] | |
| DNA NK19-1 | 27 | NK77.71_CD70_DB04_D02 | 101 (e.g., 67) |
| | | construct [no FLAG/ | |
| | | GS2; +mbIL15] | |
| AA NK19-1 | 84 (e.g., 28) | NK77.17_CD70_DB02_B06 | 102 (e.g., 68) |
| | | CAR only [no | |
| | | mbIL15/FLAG/GS2] | |
| NKG2D Chimeric Receptor DNA | 40 | NK77.58_CD70_DB03_H08 | 103 (e.g., 69) |
| | | CAR only [no | |
| | | mbIL15/FLAG/GS2] | |
| | | NK77.71_CD70_DB04_D02 | 104 (e.g., 70) |
| | | CAR only [no | |
| | | mbIL15/FLAG/GS2] | |

To facilitate cancer immunotherapies, there are also provided for herein polynucleotides, polypeptides, and vectors that encode chimeric receptors that comprise a target binding moiety (e.g., an extracellular binder of a ligand expressed by a cancer cell) and a cytotoxic signaling complex. For example, some embodiments include a polynucleotide, polypeptide, or vector that encodes, for example an activating chimeric receptor comprising an NKG2D extracellular domain that is directed against a tumor marker, for example, MICA, MICB, ULBP1, ULBP2, ULBP3, ULBP4, ULBP5, and ULBP6, among others, to facilitate targeting of an immune cell to a cancer and exerting cytotoxic effects on the cancer cell. Also provided are engineered immune cells (e.g., NK cells) expressing such chimeric receptors. There are also provided herein, in several embodiments, polynucleotides, polypeptides, and vectors that encode a construct comprising an extracellular domain comprising two or more subdomains, e.g., first and second ligand binding receptor and a cytotoxic signaling complex. Also provided are engineered immune cells (e.g., NK cells) expressing such bi-specific constructs (in some embodiments the first and second ligand binding domain target the same ligand). Methods of treating cancer and other uses of such cells for cancer immunotherapy are also provided for herein.

### Engineered Cells

In several embodiments, cells of the immune system (e.g., NK cells) are engineered to have enhanced cytotoxic effects against target cells, such as tumor cells. For example, a cell of the immune system (e.g., a NK cell) may be engineered to include a tumor-directed chimeric receptor and/or a tumor-directed CAR as described herein. Alternatively, a cell of the immune system (e.g., a NK cell) may be engineered to include a chimeric receptor directed against a cell that mediates an autoimmune disease (e.g., a B cell, T cells, and/or a plasma cell).

In several embodiments, white blood cells or leukocytes, are used, since their native function is to defend the body against growth of abnormal cells and infectious disease. There are a variety of types of white bloods cells that serve specific roles in the human immune system, and are therefore a preferred starting point for the engineering of cells disclosed herein. White blood cells include granulocytes and agranulocytes (presence or absence of granules in the cytoplasm, respectively). Granulocytes include basophils, eosinophils, neutrophils, and mast cells. Agranulocytes include lymphocytes and monocytes. Cells such as those that follow or are otherwise described herein may be engineered to include a chimeric receptor, such as an NKG2D chimeric receptor, and/or a CAR, such as a CD19-directed CAR, or a nucleic acid encoding the chimeric receptor or the CAR. In several embodiments, the cells are optionally engineered to co-express a membrane-bound interleukin 15 (mbIL15) stimulatory molecule. Thus, in several embodiments, the cells are engineered to express a CAR and a membrane-bound interleukin 15 (mbIL15). As discussed in more detail below, in several embodiments, the cells are further genetically modified to reduce and/or eliminate the alloreactivity of the cells.

### Monocytes

Monocytes are a subtype of leukocyte. Monocytes can differentiate into macrophages and myeloid lineage dendritic cells. Monocytes are associated with the adaptive immune system and serve the main functions of phagocytosis, antigen presentation, and cytokine production. Phagocytosis is the process of uptake of cellular material, or entire cells, followed by digestion and destruction of the engulfed cellular material. In several embodiments, monocytes are used in connection with one or more additional engineered cells as disclosed herein. Some embodiments of the methods and compositions described herein relate to a monocyte that expresses a CAR, or a nucleic acid encoding a CAR. Some embodiments of the methods and compositions described herein relate to a monocyte that includes a tumor-directed CAR, or a nucleic acid encoding the tumor-directed CAR. In some embodiments, the CAR is directed to a cell that mediates an autoimmune disease (e.g., a B cell, T cell, and/or a plasma cell). Several embodiments of the methods and compositions disclosed herein relate to monocytes engineered to express a CAR that targets (e.g., binds) a tumor marker, for example, CD19, CD38, CD123, CD70, Her2, mesothelin, Claudin 6, BCMA, EGFR, among any of the others disclosed herein, and a membrane-bound interleukin 15 (mbIL15) stimulatory molecule. Thus, in several embodiments, the cells are engineered to express a CAR and a membrane-bound interleukin 15 (mbIL15). Several embodiments of the methods and compositions disclosed herein relate to monocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) stimulatory molecule.

### Lymphocytes

Lymphocytes, the other primary sub-type of leukocyte include T cells (cell-mediated, cytotoxic adaptive immunity), natural killer cells (cell-mediated, cytotoxic innate immunity), and B cells (humoral, antibody-driven adaptive immunity). While B cells are engineered according to several embodiments, disclosed herein, several embodiments also relate to engineered T cells or engineered NK cells (mixtures of T cells and NK cells are used in some embodiments, either from the same donor, or different donors). Some embodiments of the methods and compositions described herein relate to a lymphocyte that expresses a CAR, or a nucleic acid encoding a CAR. In some embodiments, the CAR is directed to a cell that mediates an autoimmune disease (e.g., a B cell, T cell, and/or a plasma cell). Several embodiments of the methods and compositions disclosed herein relate to lymphocytes engineered to express a CAR that targets (e.g., binds) a tumor marker, for example, CD19, CD38, CD123, CD70, Her2, mesothelin, Claudin 6, BCMA, EGFR, among any of the others disclosed herein, and a membrane-bound interleukin 15 (mbIL15) stimulatory molecule. Thus, in several embodiments, the cells are engineered to express a CAR and a membrane-bound interleukin 15 (mbIL15). Several embodiments of the methods and compositions disclosed herein relate to lymphocytes engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) stimulatory molecule.

### T Cells

T cells are distinguishable from other lymphocytes sub-types (e.g., B cells or NK cells) based on the presence of a T-cell receptor on the cell surface. T cells can be divided into various different subtypes, including effector T cells, helper T cells, cytotoxic T cells, memory T cells, regulatory T cells, natural killer T cell, mucosal associated invariant T cells and gamma delta T cells. In some embodiments, a specific subtype of T cell is engineered. In some embodiments, a mixed pool of T cell subtypes is engineered. In some embodiments, there is no specific selection of a type of T cells to be engineered to express the cytotoxic receptor complexes disclosed herein. In several embodiments, specific techniques, such as use of cytokine stimulation are used to enhance expansion/collection of T cells with a specific marker profile. For example, in several embodiments, activation of certain human T cells, e.g. CD4+ T cells, CD8+ T cells is achieved through use of CD3 and/or CD28 as stimulatory molecules. In several embodiments, there is provided a method of treating or preventing cancer or an infectious disease, comprising administering a therapeutically effective amount of T cells expressing the cytotoxic receptor complex and/or a homing moiety as described herein. In several embodiments, the engineered T cells are autologous cells, while in some embodiments, the T cells are allogeneic cells. Some embodiments of the methods and compositions described herein relate to a T cell that expresses a CAR, or a nucleic acid encoding a CAR. In some embodiments, the CAR is directed to a cell that mediates an autoimmune disease (e.g., a B cell, T cell, and/or a plasma cell). Several embodiments of the methods and compositions disclosed herein relate to T cells engineered to express a CAR that targets (e.g., binds) a tumor marker, for example, CD19, CD38, CD123, CD70, Her2, mesothelin, Claudin 6, BCMA, EGFR, among any of the others as disclosed herein, and a membrane-bound interleukin 15 (mbIL15) stimulatory molecule. Thus, in several embodiments, the cells are engineered to express a CAR and a membrane-bound interleukin 15 (mbIL15). Several embodiments of the methods and compositions disclosed herein relate to T cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) co-stimulatory domain.

### NK Cells

In several embodiments, there is provided a method of treating or preventing a disease or condition in a subject, comprising administering to a subject having the disease or condition any of the compositions (e.g., NK cell compositions) provided herein. In some embodiments, the disease or condition is an autoimmune disease. Thus, in several embodiments, there is provided a method of treating or preventing an autoimmune disease. In some embodiments, the disease or condition is a cancer or a tumor. In several embodiments, there is provided a method of treating or preventing cancer or an infectious disease, comprising administering a therapeutically effective amount of natural killer (NK) cells expressing the cytotoxic receptor complex and/or a homing moiety as described herein. In several embodiments, the engineered NK cells are autologous cells, while in some embodiments, the NK cells are allogeneic cells. In several embodiments, the NK cells are autologous to a subject to be treated. In some embodiments, the NK cells are allogeneic to a subject to be treated.

In several embodiments, NK cells are preferred because the natural cytotoxic potential of NK cells is relatively high. In several embodiments, it is unexpectedly beneficial that the engineered cells disclosed herein can further upregulate the cytotoxic activity of NK cells, leading to an even more effective activity against target cells (e.g., tumor or other diseased cells).

Some embodiments of the methods and compositions described herein relate to NK cells engineered to express a CAR that targets (e.g., binds) an antigen expressed by target cells. In some embodiments, the antigen is CD19, CD20 CD38, CD123, CD70, HER2, mesothelin, Claudin 6, BCMA, BAFF, EGFR, or a ligand of NKG2D. In some embodiments, the antigen is CD19. In some embodiments, the antigen is CD20. In some embodiments, the antigen is CD38. In some embodiments, the antigen is CD123. In some embodiments, the antigen is CD70. In some embodiments, the antigen is HER2. In some embodiments, the antigen is mesothelin. In some embodiments, the antigen is Claudin 6. In some embodiments, the antigen is BCMA. In some embodiments, the antigen is BAFF. In some embodiments, the antigen is CD38. EGFR. In some embodiments, the antigen is a NKG2D ligand. In some embodiments, the target cells comprise B cells. For example, in some embodiments, the target cells are B cells and the CAR binds to CD19. In some embodiments, the target cells comprise T cells. In some embodiments, the target cells comprise plasma cells. For example, in some embodiments, the target cells are plasma cells and the CAR binds to BCMA.

In some embodiments, the target cells comprise an autoimmune disease. Thus, in some embodiments of the methods and compositions described herein relate to NK cells engineered to express a CAR that targets (e.g., binds) a cell that mediates an autoimmune disease (e.g., a B cell and/or a plasma cell). In some embodiments of the methods and compositions described herein, the NK cells are engineered to express a CAR that targets (e.g., binds) a B cell. For example, in some embodiments, the CAR is an anti-CD19 CAR. In some embodiments of the methods and compositions described herein, the NK cells are engineered to express a CAR that targets (e.g., binds) a plasma cell. For example, in some embodiments, the CAR is an anti-BCMA CAR. In several embodiments, the cells are also engineered to express a membrane-bound interleukin 15 (mbIL15). Thus, in several embodiments, the cells are engineered to express a CAR and a membrane-bound interleukin 15 (mbIL15).

In some embodiments, the target cells comprise cancer or tumor cells. Thus, in some embodiments of the methods and compositions described herein relate to NK cells engineered to express a CAR that targets (e.g., binds) a cancer or tumor marker. Some embodiments of the methods and compositions described herein relate to NK cells engineered to express a CAR that targets (e.g., binds) a tumor marker, for example, CD19, CD38, CD123, CD70, Her2, mesothelin, Claudin 6, BCMA, EGFR, among any of the others disclosed herein, and optionally a membrane-bound interleukin 15 (mbIL15) stimulatory molecule. In some embodiments, the NK cells are engineered to express a membrane-bound IL15 (mbIL15). Thus, in several embodiments, the cells are engineered to express a CAR and a membrane-bound interleukin 15 (mbIL15). Several embodiments of the methods and compositions disclosed herein relate to NK cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) stimulatory molecule.

In some embodiments, the NK cells are primary NK cells. In some embodiments, the NK cells are obtained from a donor. In some embodiments, the NK cells are obtained from a blood sample (e.g., a peripheral blood or cord blood sample) from a donor. In some embodiments, the NK cells are obtained from a peripheral blood sample from a donor. In some embodiments, the NK cells are derived from cell line NK-92. NK-92 cells are derived from NK cells, but lack major inhibitory receptors displayed by normal NK cells, while retaining the majority of activating receptors. Some embodiments of NK-92 cells described herein related to NK-92 cell engineered to silence certain additional inhibitory receptors, for example, SMAD3, allowing for upregulation of interferon-γ (IFNγ), granzyme B, and/or perforin production. Additional information relating to the NK-92 cell line is disclosed in WO 1998/49268 and U.S. Patent Application Publication No. 2002-0068044 and incorporated in their entireties herein by reference. NK-92 cells are used, in several embodiments, in combination with one or more of the other cell types disclosed herein. For example, in one embodiment, NK-92 cells are used in combination with NK cells as disclosed herein. In an additional embodiment, NK-92 cells are used in combination with T cells as disclosed herein.

### Hematopoietic Stem Cells

In some embodiments, hematopoietic stem cells (HSCs) are used in the methods of immunotherapy disclosed herein. In several embodiments, the cells are engineered to express a homing moiety and/or a cytotoxic receptor complex. HSCs are used, in several embodiments, to leverage their ability to engraft for long-term blood cell production, which could result in a sustained source of targeted anti-cancer effector cells, for example to combat cancer remissions. In several embodiments, this ongoing production helps to offset anergy or exhaustion of other cell types, for example due to the tumor microenvironment. In several embodiments allogeneic HSCs are used, while in some embodiments, autologous HSCs are used. In several embodiments, HSCs are used in combination with one or more additional engineered cell type disclosed herein. Some embodiments of the methods and compositions described herein relate to a HSC that expresses a CAR, or a nucleic acid encoding a CAR. In some embodiments, the CAR is directed to a cell that mediates an autoimmune disease (e.g., a B cell, T cell, and/or a plasma cell). Some embodiments of the methods and compositions described herein relate to a stem cell, such as a hematopoietic stem cell engineered to express a CAR that targets (e.g., binds) a tumor marker, for example, CD19, CD38, CD123, CD70, Her2, mesothelin, Claudin 6, BCMA, EGFR, among any of the others disclosed herein, and optionally a membrane-bound interleukin 15 (mbIL15) stimulatory molecule. Thus, in several embodiments, the cells are engineered to express a CAR and a membrane-bound interleukin 15 (mbIL15). Several embodiments of the methods and compositions disclosed herein relate to hematopoietic stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) stimulatory molecule.

### Induced Pluripotent Stem Cells

In some embodiments, induced pluripotent stem cells (iPSCs) are used in the method of immunotherapy disclosed herein. iPSCs are used, in several embodiments, to leverage their ability to differentiate and derive into non-pluripotent cells, including, but not limited to, CD34 cells, hemogenic endothelium cells, HSCs (hematopoietic stem and progenitor cells), hematopoietic multipotent progenitor cells, T cell progenitors, NK cell progenitors, T cells, NKT cells, NK cells, and B cells comprising one or several genetic modifications at selected sites through differentiating iPSCs or less differentiated cells comprising the same genetic modifications at the same selected sites. In several embodiments, the iPSCs are used to generate iPSC-derived NK cells. In several embodiments, the cells are engineered to express a homing moiety and/or a cytotoxic receptor complex. In several embodiments, iPSCs are used in combination with one or more additional engineered cell type disclosed herein. Some embodiments of the methods and compositions described herein relate to an iPSC that expresses a CAR, or a nucleic acid encoding a CAR. In some embodiments, the CAR is directed to a cell that mediates an autoimmune disease (e.g., a B cell, T cell, and/or a plasma cell). Some embodiments of the methods and compositions described herein relate to a stem cell, such as a induced pluripotent stem cell engineered to express a CAR that targets (e.g., binds) a tumor marker, for example, CD19, CD38, CD123, CD70, Her2, mesothelin, Claudin 6, BCMA, EGFR, among any of the others disclosed herein, and optionally a membrane-bound interleukin 15 (mbIL15) stimulatory molecule. Thus, in several embodiments, the cells are engineered to express a CAR and a membrane-bound interleukin 15 (mbIL15). Several embodiments of the methods and compositions disclosed herein relate to induced pluripotent stem cells engineered to express an activating chimeric receptor that targets a ligand on a tumor cell, for example, MICA, MICB, ULBP1, ULBP2, ULBP3, ULBP4, ULBP5, and ULBP6 (among others) and optionally a membrane-bound interleukin 15 (mbIL15) stimulatory molecule.

### IV. Genetic Editing of Cells

As discussed herein, a variety of cell types can be utilized in cellular immunotherapy. Several embodiments disclosed herein also relate to the identification of donor cells that are particularly disposed to expansion in culture or exhibit particularly robust cytotoxicity against target cells. For example, in some instances, the methods include selecting donors with NK cells, or enriching for NK cells from donor samples, that exhibit superior expansion in culture. Several embodiments disclosed herein also relate to the identification of donors whose cells are particularly disposed to efficient expansion in culture or exhibit particularly robust cytotoxicity against target tumor cells (e.g., when engineered to express a CAR). Further, genetic modifications can be made to these cells in order to enhance one or more aspects of their efficacy (e.g., cytotoxicity) and/or persistence (e.g., active life span). As discussed herein, in several embodiments NK cells are used for immunotherapy. In several embodiments provided for herein, gene editing of the NK cell can advantageously impart to the edited NK cell the ability to resist and/or overcome various inhibitory signals that are generated in the tumor microenvironment. It is known that tumors generate a variety of signaling molecules that are intended to reduce the anti-tumor effects of immune cells. As discussed in more detail below, in several embodiments, gene editing of the NK cell limits this tumor microenvironment suppressive effect on the NK cells, T cells, combinations of NK and T cells, or any edited/engineered immune cell provided for herein. As discussed below, in several embodiments, gene editing is employed to reduce or knockout expression of target proteins, for example by disrupting the underlying gene encoding the protein. In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, the gene is completely knocked out, such that expression of the target protein is undetectable. In several embodiments, gene editing is used to “knock in” or otherwise enhance expression of a target protein. In several embodiments, expression of a target protein can be enhanced by about 30%, about 40%, about 50%, about 60%, about 70%, about 75%, about 80%, about 85%, about 90%, about 95%, about 97%, about 98%, about 99%, or more (including any amount between those listed). Unless indicated otherwise to the contrary, the sequences provided for guide RNAs that are recited using deoxyribonucleotides refer to the target DNA and shall be considered as also referencing those guides used in practice (e.g., employing ribonucleotides, where the ribonucleotide uracil is used in lieu of deoxyribonucleotide thymine or vice-versa where thymine is used in lieu of uracil, wherein both are complementary base pairs to adenine when reciting either an RNA or DNA sequence). For example, a gRNA with the sequence ATGCTCAATGCGTC (SEQ ID NO:105) shall also refer to the following sequence AUGCUCAAUGCGUC (SEQ ID NO:106) or a gRNA with sequence AUGCUCAAUGCGUC (SEQ ID NO:106) shall also refer to the following sequence ATGCTCAATGCGTC (SEQ ID NO:105). Further, the non-target DNA sequence to which a particular gRNA sequence binds is complementary to the sequence of the particular gRNA. For example, a gRNA with the provided sequence of ATGCTCAATGCGTC (SEQ ID NO:105) binds to a non-target DNA sequence of TACGAGTTACGCAG (SEQ ID NO: 107). In this situation, the corresponding target DNA sequence, which is complementary to the non-target DNA sequence, is ATGCTCAATGCGTC (SEQ ID NO:105).

By way of non-limiting example, modulators of one or more aspects of NK cell function are modulated through gene editing. A variety of cytokines impart either negative (as with TGF-beta in more detail below) or positive signals to immune cells. By way of non-limiting example, IL15 is a positive regulator of NK cells, which as disclosed herein, can enhance one or more of NK cell homing, NK cell migration, NK cell expansion/proliferation, NK cell cytotoxicity, and/or NK cell persistence. To keep NK cells in check under normal physiological circumstances, a cytokine-inducible SH2-containing protein (CIS, encoded by the CISH gene) acts as a critical negative regulator of IL-15 signaling in NK cells. As discussed herein, because IL 15 biology impacts multiple aspects of NK cell functionality, including, but not limited to, proliferation/expansion, activation, cytotoxicity, persistence, homing, migration, among others. Thus, according to several embodiments, editing CISH enhances the functionality of NK cells across multiple functionalities, leading to a more effective and long-lasting NK cell therapeutic. In several embodiments, inhibitors of CIS are used in conjunction with engineered NK cell administration. In several embodiments, the CIS expression is knocked down or knocked out through gene editing of the CISH gene, for example, by use of CRISPR-Cas editing. Small interfering RNA, antisense RNA, TALENs or zinc fingers are used in other embodiments. In some embodiments CIS expression in T cells is knocked down through gene editing. Non-limiting examples of guide RNAs that can target an endonuclease, such as Cas9, to edit a CISH gene are provided in Table 2, below (additional information on CISH editing can be found, for example in International Patent Application No. PCT/US2020/035752, which is incorporated in its entirety by reference herein). Additional non-limiting examples of CISH-targeting guide RNAs are described in PCT Application No. PCT/US2023/071537, which is incorporated herein by reference in its entirety.

TABLE 2
CISH Guide RNAs

| SEQ ID NO: | Name | Sequence | Target |
|---|---|---|---|
| 29 | CISH-1 | CTCACCAGATTCCCGAAGGT | Exon 2 |
| 30 | CISH-2 | CCGCCTTGTCATCAACCGTC | Exon 3 |
| 31 | CISH-3 | TCTGCGTTCAGGGGTAAGCG | Exon 1 |
| 32 | CISH-4 | GCGCTTACCCCTGAACGCAG | Exon 1 |
| 33 | CISH-5 | CGCAGAGGACCATGTCCCCG | Exon 1 |

In several embodiments, CISH gene editing endows an NK cell with enhanced ability to home to a target site. In several embodiments, CISH gene editing endows an NK cell with enhanced ability to migrate, e.g., within a tissue in response to, for example chemoattractants or away from repellants. In several embodiments, CISH gene editing endows an NK cell with enhanced ability to be activated, and thus exert, for example, anti-tumor effects. In several embodiments, CISH gene editing endows an NK cell with enhanced proliferative ability, which in several embodiments, allows for generation of robust NK cell numbers from a donor blood sample. In addition, in such embodiments, NK cells edited for CISH and engineered to express a CAR are more readily, robustly, and consistently expanded in culture. In several embodiments, CISH gene editing endows an NK cell with enhanced cytotoxicity. In several embodiments, the editing of CISH synergistically enhances the cytotoxic effects of engineered NK cells that express a CAR.

In several embodiments, CISH gene editing activates or inhibits a wide variety of pathways. The CIS protein is a negative regulator of IL15 signaling by way of, for example, inhibiting JAK-STAT signaling pathways. These pathways would typically lead to transcription of IL15-responsive genes (including CISH). In several embodiments, knockdown of CISH disinhibits JAK-STAT (e.g., JAK1-STAT5) signaling and there is enhanced transcription of IL15-responsive genes. In several embodiments, knockout of CISH yields enhanced signaling through mammalian target of rapamycin (mTOR), with corresponding increases in expression of genes related to cell metabolism and respiration. In several embodiments, knockout of CISH yields IL15 induced increased expression of IL-2Rα (CD25), but not IL-15Rα or IL-2/15Rβ, enhanced NK cell membrane binding of IL15 and/or IL2, increased phosphorylation of STAT-3 and/or STAT-5, and elevated expression of the antiapoptotic proteins, such as Bcl-2. In several embodiments, CISH knockout results in IL15-induced upregulation of selected genes related to mitochondrial functions (e.g., electron transport chain and cellular respiration) and cell cycle. Thus, in several embodiments, knockout of CISH by gene editing enhances the NK cell cytotoxicity and/or persistence, at least in part via metabolic reprogramming. In several embodiments, negative regulators of cellular metabolism, such as TXNIP, are downregulated in response to CISH knockout. In several embodiments, promotors for cell survival and proliferation including BIRC5 (Survivin), TOP2A, CKS2, and RACGAP1 are upregulated after CISH knockout, whereas antiproliferative or proapoptotic proteins such as TGFB1, ATM, and PTCH1 are downregulated. In several embodiments, CISH knockout alters the state (e.g., activates or inactivates) signaling via or through one or more of CXCL-10, IL2, TNF, IFNg, IL13, IL4, Jnk, PRF1, STAT5, PRKCQ, IL2 receptor Beta, SOCS2, MYD88, STAT3, STAT1, TBX21, LCK, JAK3, IL& receptor, ABL1, IL9, STAT5A, STAT5B, Tcf7, PRDM1, and/or EOMES.

In several embodiments, gene editing reduces transcription of CISH by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CISH by at least about 30%. In several embodiments, gene editing reduces transcription of CISH by at least about 40%. In several embodiments, gene editing reduces transcription of CISH by at least about 50%. In several embodiments, gene editing reduces transcription of CISH by at least about 60%. In several embodiments, gene editing reduces transcription of CISH by at least about 70%. In several embodiments, gene editing reduces transcription of CISH by at least about 80%. In several embodiments, gene editing reduces transcription of CISH by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CIS by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of CIS by at least about 30%. In several embodiments, gene editing reduces expression of CIS by at least about 40%. In several embodiments, gene editing reduces expression of CIS by at least about 50%. In several embodiments, gene editing reduces expression of CIS by at least about 60%. In several embodiments, gene editing reduces expression of CIS by at least about 70%. In several embodiments, gene editing reduces expression of CIS by at least about 80%. In several embodiments, gene editing reduces expression of CIS by at least about 90%.

In several embodiments, gene editing of the immune cells (e.g., NK cells) can also provide unexpected enhancement in the expansion, persistence and/or cytotoxicity of the edited immune cell. As disclosed herein, engineered cells (e.g., those expressing a CAR) may also be edited, the combination of which provides for a robust cell for immunotherapy. In several embodiments, the edits allow for unexpectedly improved NK cell expansion, persistence and/or cytotoxicity. In several embodiments, knockout of CISH expression in NK cells removes a potent negative regulator of IL15-mediated signaling in NK cells, disinhibits the NK cells and allows for one or more of enhanced NK cell homing, NK cell migration, activation of NK cells, expansion, cytotoxicity and/or persistence. Additionally, in several embodiments, the editing can enhance NK cell function in the otherwise suppressive tumor microenvironment. In several embodiments, CISH gene editing results in enhanced NK cell expansion, persistence and/or cytotoxicity without requiring Notch ligand being provided exogenously.

Casitas B-lineage lymphoma-b (Cbl-b) is an E3 ubiquitin ligase that negatively regulates T cell activation. Loss of expression of Cbl-b in NK cells increases antitumor immunity. Moreover, Cbl-b deficient T cells and NK cells are resistant to PD-L1/PD-1 mediated suppression (Fujiwara et al., Front. Immunol. (2017) 8:42). Thus, according to several embodiments, genetic editing of Cbl-b (e.g., knockdown or knockout) increases the cytotoxicity, persistence, and/or otherwise enhances the efficacy of NK cells as disclosed herein.

In several embodiments, the expression of Cbl-b is reduced and/or eliminated in order to increase overall activation in resultant NK cells, or other cell type provided for herein. In several embodiments, the BCLB gene is disrupted and/or knocked out using one or more of the gene editing methods disclosed herein. In several embodiments, the CBLB gene is disrupted and/or knocked out using a Crispr-Cas mediated approach (e.g., Cas9), or other guided nuclease as disclosed elsewhere herein. Non-limiting examples of CBLB-targeting guide RNAs are described in PCT Application No. PCT/US2023/071537, which is incorporated herein by reference in its entirety.

In several embodiments, gene editing reduces transcription of CBLB by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of CBLB by at least about 30%. In several embodiments, gene editing reduces transcription of CBLB by at least about 40%. In several embodiments, gene editing reduces transcription of CBLB by at least about 50%. In several embodiments, gene editing reduces transcription of CBLB by at least about 60%. In several embodiments, gene editing reduces transcription of CBLB by at least about 70%. In several embodiments, gene editing reduces transcription of CBLB by at least about 80%. In several embodiments, gene editing reduces transcription of CBLB by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of Cbl-b by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of Cbl-b by at least about 30%. In several embodiments, gene editing reduces expression of Cbl-b by at least about 40%. In several embodiments, gene editing reduces expression of Cbl-b by at least about 50%. In several embodiments, gene editing reduces expression of Cbl-b by at least about 60%. In several embodiments, gene editing reduces expression of Cbl-b by at least about 70%. In several embodiments, gene editing reduces expression of Cbl-b by at least about 80%. In several embodiments, gene editing reduces expression of Cbl-b by at least about 90%.

Mediator complex subunit 12 (MED12) is one part of the four-part cyclin dependent kinase (CDK) module of Mediator along with MED13, cyclin-dependent kinase 8 (CDK8) and cyclin C (CCNC). Mutations in MED12 have been associated with lymphoproliferative disorders (Kampjarvi et al., Oncotarget (2015) 6(3):1884-88). More recently, targeted deletion of MED12 in human CAR T cells was observed to increase proliferation, cytokine production, and antitumor activity. In particular, MED12 deficient T cells exhibited changes at genes regulating effector T cell differentiation. See Freitas et al., Cancer Res (2022) 82(12_suppl):2822; and Freitas et al., Science (2022) 378(6620):eabn5647. It is contemplated that reductions in MED12 could provide NK cells with an enhanced persistence, allowing cytotoxicity against target tumor cells. In several embodiments, MED12 expression is disrupted and/or knocked out using a Crispr-Cas mediated approach (e.g., Cas9), Non-limiting examples of MED12-targeting guide RNAs are described in PCT Application No. PCT/US2023/071537, which is incorporated herein by reference in its entirety.

In several embodiments, gene editing reduces transcription of MED12 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces transcription of MED12 by at least about 30%, In several embodiments, gene editing reduces transcription of MED12 by at least about 40%, In several embodiments, gene editing reduces transcription of MED12 by at least about 50%, In several embodiments, gene editing reduces transcription of MED12 by at least about 60%, In several embodiments, gene editing reduces transcription of MED12 by at least about 70%, In several embodiments, gene editing reduces transcription of MED12 by at least about 80%, In several embodiments, gene editing reduces transcription of MED12 by at least about 90%.

In several embodiments, gene editing can reduce expression of a target protein by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MED12 by about 30%, about 40%, about 50%, about 60%, about 70%, about 75%, about 80%, about 85%, about 90%, about 95%, about 97%, about 98%, about 99%, or more (including any amount between those listed). In several embodiments, gene editing reduces expression of MED12 by at least about 30%, In several embodiments, gene editing reduces expression of MED12 by at least about 40%, In several embodiments, gene editing reduces expression of MED12 by at least about 50%, In several embodiments, gene editing reduces expression of MED12 by at least about 60%, In several embodiments, gene editing reduces expression of MED12 by at least about 70%, In several embodiments, gene editing reduces expression of MED12 by at least about 80%, In several embodiments, gene editing reduces expression of MED12 by at least about 90%.

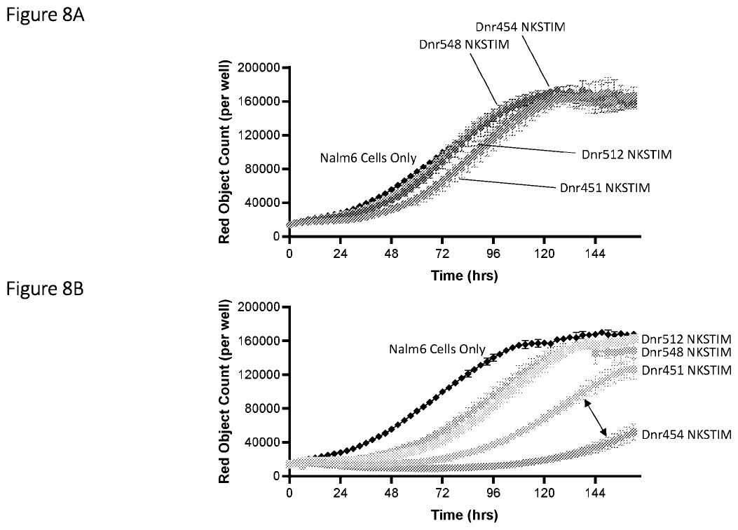

As mentioned above, TGF-beta is one a cytokine released by tumor cells that results in immune suppression within the tumor microenvironment. That immune suppression reduces the ability of immune cells, even engineered CAR-immune cells is some cases, to destroy the tumor cells, thus allowing for tumor progression. In several embodiments, as discussed in detail below, immune checkpoint inhibitors are disrupted through gene editing. In several embodiments, blockers of immune suppressing cytokines in the tumor microenvironment are used, including blockers of their release or competitive inhibitors that reduce the ability of the signaling molecule to bind and inhibit an immune cell. Such signaling molecules include, but are not limited to TGF-beta, IL10, arginase, inducible NOS, reactive-NOS, Arg1, Indoleamine 2,3-dioxygenase (IDO), and PGE2. However, in additional embodiments, there are provided immune cells, such as NK cells, wherein the ability of the NK cell (or other cell) to respond to a given immunosuppressive signaling molecule is disrupted and/or eliminated. For example, in several embodiments, in several embodiments, NK cells are genetically edited to become have reduced sensitivity to TGF-beta. TGF-beta is an inhibitor of NK cell function on at least the levels of proliferation and cytotoxicity. See, for example, FIG. 8A which schematically shows some of the inhibitory pathways by which TGF-beta reduces NK cell activity and/or proliferation. Thus, according to some embodiments, the expression of the TGF-beta receptor is knocked down or knocked out through gene editing, such that the edited NK is resistant to the immunosuppressive effects of TGF-beta in the tumor microenvironment. In several embodiments, the TGFB2 receptor is knocked down or knocked out through gene editing, for example, by use of CRISPR-Cas editing. Small interfering RNA, antisense RNA, TALENs or zinc fingers are used in other embodiments. Other isoforms of the TGF-beta receptor (e.g., TGF-beta 1 and/or TGF-beta 3) are edited in some embodiments. In some embodiments TGF-beta receptors in T cells are knocked down through gene editing. Non-limiting examples of guide RNAs that can target an endonuclease, such as Cas9, to edit a TGFBR2 gene are provided in Table 3, below (additional information on TGFBR editing can be found, for example in International Patent Application No. PCT/US2020/035752, which is incorporated in its entirety by reference herein).

TABLE 3
TGFb Receptor Type 2 Isoform Guide RNAs

| SEQ ID NO: | Name | Sequence | Target |
|---|---|---|---|
| 34 | TGFBR2-1 | CCCCTACCATGACTTTATTC | Exon 4 |
| 35 | TGFBR2-2 | ATTGCACTCATCAGAGCTAC | Exon 4 |
| 36 | TGFBR2-3 | AGTCATGGTAGGGGAGCTTG | Exon 4 |
| 37 | TGFBR2-4 | TGCTGGCGATACGCGTCCAC | Exon 1 |
| 38 | TGFBR2-5 | GTGAGCAATCCCCCGGGCGA | Exon 4 |
| 39 | TGFBR2-6 | AACGTGCGGTGGGATCGTGC | Exon 1 |

In several embodiments, genetic editing (whether knock out or knock in) of any of the target genes (e.g., CISH, TGFBR2, or any other target gene disclosed in International Patent Application No. PCT/US2020/035752, U.S. Provisional Application No. 63/121,206, or U.S. Provisional Application No. 63/201,159, each of which is incorporated by reference herein in its entirety), is accomplished through targeted introduction of DNA breakage, and subsequent DNA repair mechanism. In several embodiments, double strand breaks of DNA are repaired by non-homologous end joining (NHEJ), wherein enzymes are used to directly join the DNA ends to one another to repair the break. In several embodiments, however, double strand breaks are repaired by homology directed repair (HDR), which is advantageously more accurate, thereby allowing sequence specific breaks and repair. HDR uses a homologous sequence as a template for regeneration of missing DNA sequences at the break point, such as a vector with the desired genetic elements (e.g., an insertion element to disrupt the coding sequence of a TCR) within a sequence that is homologous to the flanking sequences of a double strand break. This will result in the desired change (e.g., insertion) being inserted at the site of the DSB.

In several embodiments, gene editing is accomplished by one or more of a variety of engineered nucleases. In several embodiments, restriction enzymes are used, particularly when double strand breaks are desired at multiple regions. In several embodiments, a bioengineered nuclease is used. Depending on the embodiment, one or more of a Zinc Finger Nuclease (ZFN), transcription-activator like effector nuclease (TALEN), meganuclease and/or clustered regularly interspaced short palindromic repeats (CRISPR/Cas9) system are used to specifically edit the genes encoding one or more of the TCR subunits.

Meganucleases are characterized by their capacity to recognize and cut large DNA sequences (from 14 to 40 base pairs). In several embodiments, a meganuclease from the LAGLIDADG family is used, and is subjected to mutagenesis and screening to generate a meganuclease variant that recognizes a unique sequence(s), such as a specific site in the TCR, or CISH, or any other target gene disclosed herein. Target sites in the TCR can readily be identified. Further information of target sites within a region of the TCR can be found in US Patent Publication No. 2018/0325955, and US Patent Publication No. 2015/0017136, each of which is incorporated by reference herein in its entirety. In several embodiments, two or more meganucleases, or functions fragments thereof, are fused to create a hybrid enzyme that recognizes a desired target sequence within the target gene (e.g., CISH).

In contrast to meganucleases, ZFNs and TALEN function based on a non-specific DNA cutting catalytic domain which is linked to specific DNA sequence recognizing peptides such as zinc fingers or transcription activator-like effectors (TALEs). Advantageously, the ZFNs and TALENs thus allow sequence-independent cleavage of DNA, with a high degree of sequence-specificity in target recognition. Zinc finger motifs naturally function in transcription factors to recognize specific DNA sequences for transcription. The C-terminal part of each finger is responsible for the specific recognition of the DNA sequence. While the sequences recognized by ZFNs are relatively short, (e.g., ~3 base pairs), in several embodiments, combinations of 2, 3, 4, 5, 6, 7, 8, 9, 10 or more zinc fingers whose recognition sites have been characterized are used, thereby allowing targeting of specific sequences, such as a portion of the TCR (or an immune checkpoint inhibitor). The combined ZFNs are then fused with the catalytic domain(s) of an endonuclease, such as FokI (optionally a FokI heterodimer), in order to induce a targeted DNA break. Additional information on uses of ZFNs to edit the TCR and/or immune checkpoint inhibitors can be found in U.S. Pat. No. 9,597,357, which is incorporated by reference herein.

Transcription activator-like effector nucleases (TALENs) are specific DNA-binding proteins that feature an array of 33 or 34-amino acid repeats. Like ZFNs, TALENs are a fusion of a DNA cutting domain of a nuclease to TALE domains, which allow for sequence-independent introduction of double stranded DNA breaks with highly precise target site recognition. TALENs can create double strand breaks at the target site that can be repaired by error-prone non-homologous end-joining (NHEJ), resulting in gene disruptions through the introduction of small insertions or deletions. Advantageously, TALENs are used in several embodiments, at least in part due to their higher specificity in DNA binding, reduced off-target effects, and ease in construction of the DNA-binding domain.

CRISPRs (Clustered Regularly Interspaced Short Palindromic Repeats) are genetic elements that bacteria use as protection against viruses. The repeats are short sequences that originate from viral genomes and have been incorporated into the bacterial genome. Cas (CRISPR associated proteins) process these sequences and cut matching viral DNA sequences. By introducing plasmids containing Cas genes and specifically constructed CRISPRs into eukaryotic cells, the eukaryotic genome can be cut at any desired position. Additional information on CRISPR can be found in US Patent Publication No. 2014/0068797, which is incorporated by reference herein. In several embodiments, CRISPR is used to manipulate the gene(s) encoding a target gene to be knocked out or knocked in, for example CISH, TGFBR2, TCR, B2M, CIITA, CD47, HLA-E, etc. In several embodiments, CRISPR is used to edit one or more of the TCRs of a T cell and/or the genes encoding one or more immune checkpoint inhibitors. In several embodiments, the immune checkpoint inhibitor is selected from one or more of CTLA4 and PD1. In several embodiments, CRISPR is used to truncate one or more of TCRα, TCRβ, TCRγ, and TCRδ. In several embodiments, a TCR is truncated without impacting the function of the CD3z signaling domain of the TCR. Depending on the embodiment and which target gene is to be edited, a Class 1 or Class 2 Cas is used. In several embodiments, a Class 1 Cas is used and the Cas type is selected from the following types: I, IA, IB, IC, ID, IE, IF, IU, III, IIIA, IIIB, IIIC, IIID, IV IVA, IVB, and combinations thereof. In several embodiments, the Cas is selected from the group consisting of Cas3, Cas8a, Cas5, Cas8b, Cas8c, Cas10d, Cse1, Cse2, Csy1, Csy2, Csy3, GSU0054, Cas10, Csm2, Cmr5, Cas10, Csx11, Csx10, Csf1, and combinations thereof. In several embodiments, a Class 2 Cas is used and the Cas type is selected from the following types: II, IIA, IIB, IIC, V, VI, and combinations thereof. In several embodiments, the Cas is selected from the group consisting of Cas9, Csn2, Cas4, Cpf1, C2c1, C2c3, Cas13a (previously known as C2c2), Cas13b, Cas13c, CasX, CasY and combinations thereof. In some embodiments, class 2 CasX is used, wherein CasX is capable of forming a complex with a guide nucleic acid and wherein the complex can bind to a target DNA, and wherein the target DNA comprises a non-target strand and a target strand. In some embodiments, class 2 CasY is used, wherein CasY is capable of binding and modifying a target nucleic acid and/or a polypeptide associated with target nucleic acid.

In several embodiments, as discussed above, editing of CISH advantageously imparts to the edited cells, particularly edited NK cells, enhanced expansion, cytotoxicity and/or persistence. Additionally, in several embodiments, the modification of the TCR comprises a modification to TCRα, but without impacting the signaling through the CD3 complex, allowing for T cell proliferation. In one embodiment, the TCRα is inactivated by expression of pre-Ta in the cells, thus restoring a functional CD3 complex in the absence of a functional alpha/beta TCR. As disclosed herein, the non-alloreactive modified T cells are also engineered to express a CAR to redirect the non-alloreactive T cells specificity towards tumor marker, but independent of MHC. Combinations of editing are used in several embodiments, such as knockout of the TCR and CISH in combination, or knock out of CISH and knock in of CD47, by way of non-limiting examples. In several embodiments, the gene edit to reduce/eliminate expression of a target gene is performed prior to expanding the cells in culture. In several embodiments, the gene edit to reduce/eliminate expression of a target gene is performed prior to genetically engineering the cells to express a chimeric receptor (e.g., CAR). In several embodiments, the gene edit to reduce/eliminate expression of, for example, CISH, is performed prior to expanding the cells in culture. For example, in several embodiments, the cells to be expanded are edited at least 12 hours, at least 18 hours, at least 24 hours, at least 36 hours, or at least 48 hours prior to expansion.

### V. Cells to Facilitate Expansion of Immune Cells

In several embodiments, cell lines are used in a co-culture with a population of immune cells that are to be expanded. Such cell lines are referred to herein as “stimulatory cells,” which can also be referred to as “feeder cells”. In several embodiments, the entire population of immune cells is to be expanded, while in several embodiments, a selected immune cell subpopulation is to be expanded. For example, in several embodiments, NK cells are expanded relative to other immune cell subpopulations (such as T cells). In other embodiments, both NK cells and T cells are expanded. In several embodiments, the feeder cells are themselves genetically modified. In some embodiments, the feeder cells do not express MHC I molecules, which have an inhibitory effect on NK cells. In some embodiments, the feeder cells need not entirely lack MHC I expression, however they may express MHC I molecules at a lower level than a wild type cell. For example, in several embodiments, if a wild type cell expresses an MHC at a level of X, the cell lines used may express MHC at a level less than 95% of X, less than 90% of X, less than 85% of X, less than 80% of X, less than 70% of X, less than 50% of X, less than 25% of X, and any expression level between (and including) those listed. In several embodiments, the stimulatory cells are immortalized, e.g., a cancer cell line. However, in several embodiments, the stimulatory cells are primary cells.

Various cell types can be used as feeder cells, depending on the embodiment. These include, but are not limited to, K562 cells, certain Wilm's Tumor cell lines (for example Wilms tumor cell line HFWT), endometrial tumor cells (for example, HHUA), melanoma cells (e.g., HMV-II), hepatoblastoma cells (e.g., HuH-6), lung small cell carcinoma cells (e.g., Lu-130 and Lu-134-A), neuroblastoma cells (e.g., NB19 and NB69), embryonal carcinoma testis cells (e.g., NEC14), cervical carcinoma cells (TCO-2), neuroblastoma cells (e.g., TNB1), 721.221 EBV transformed B cell line, among others.

In additional embodiments, the feeder cells also have reduced (or lack) MHC II expression, as well as having reduced (or lacking) MHC I expression. In some embodiments, other cell lines that may initially express MHC class I molecules can be used, in conjunction with genetic modification of those cells to reduce or knock out MHC I expression. Genetic modification can be accomplished through the use of gene editing techniques (e.g. a Crispr/Cas system; RNA editing with an Adenosine deaminases acting on RNA (ADAR), zinc fingers, TALENS, etc.), inhibitory RNA (e.g., siRNA), or other molecular methods to disrupt and/or reduce the expression of MHC I molecules on the surface of the cells.

As discussed in more detail below, in several embodiments, the feeder cells are engineered to express certain stimulatory molecules (e.g. interleukins, CD3, 4-1BBL, etc.) to promote immune cell expansion and activation. Engineered feeder cells are disclosed in, for example, International Patent Application PCT/SG2018/050138, which is incorporated in its entirety by reference herein. In several embodiments, the stimulatory molecules, such as interleukin 12, 18, and/or 21 are separately added to the co-culture media, for example at defined times and in particular amounts, to effect an enhanced expansion of a desired sub-population(s) of immune cells.

### VI. Stimulatory Molecules to Facilitate Expansion of Immune Cells

As discussed briefly above, certain molecules promote the expansion of immune cells, such as NK cells, including engineered NK, and also cells that have optionally been genetically edited. Depending on the embodiment, the stimulatory molecule, or molecules, can be expressed on the surface of the feeder cells used to expand the immune population. For example, in several embodiments a feeder cell population (e.g., K562 cells) is engineered to express 4-1BBL. In several embodiments a feeder cell population (e.g., K562 cells) is engineered to express membrane-bound interleukin 15 (mbIL15). For example, in several embodiments a K562 feeder cell population is engineered to express 4-1BBL and/or membrane bound interleukin 15 (mbIL15). In several embodiments a feeder cell population (e.g., K562 cells) is engineered to express 4-1BBL and mbIL15. Additional embodiments relate to further membrane bound interleukins or stimulatory agents. Examples of such additional membrane bound stimulatory molecules can be found in International Patent Application PCT/SG2018/050138 and additional information on stimulating agents can be found in International Patent Application No. PCT/US2020/044033, each of which is incorporated in its entirety by reference herein.

In several embodiments, the methods disclosed herein relate to addition of one or more stimulatory molecules to the culture media in which engineered feeder cells and engineered NK cells are co-cultured. As discussed above, the cells may also be genetically edited. The editing and engineering may be performed in any order, however, in several embodiments, the cells are first edited, then subject to expansion for a period of time, with the engineering (e.g., to yield expression of a CAR) being performed during the expansion. In several embodiments, one or more interleukins is added. For example, in several embodiments, IL2 is added to the media. In several embodiments, IL12 is added to the media. In several embodiments, IL18 is added to the media. In several embodiments, IL21 is added to the media. In several embodiments, combinations of two or more of IL2, IL12, IL18, and/or IL21 is added to the media. In some embodiments, rather than using a feeder cell with mbIL15, soluble IL15 is added to the media (alone or in combination with any of IL2, IL12, IL18, and IL21).

In several embodiments, the media comprises one or more vitamin, inorganic salt and/or amino acids. In several embodiments, the media comprises 1, 2, 3, 4, 5, 6, 7, 8, 9, 10 or all of Glycine, L-Arginine, L-Asparagine, L-Aspartic acid, L-Cystine (e.g., L-Cystine 2HCl), L-Glutamic Acid, L-Glutamine, L-Histidine, L-Hydroxyproline, L-Isoleucine, L-Leucine, L-Lysine hydrochloride, L-Methionine, L-Phenylalanine, L-Proline, L-Serine, L-Threonine L-Tryptophan, L-Tyrosine (e.g., L-Tyrosine disodium salt dehydrate), and L-Valine. In several embodiments, the media comprises 1, 2, 3, 4, or more of Biotin, Choline chloride, D-Calcium pantothenate, Folic Acid, i-Inositol, Niacinamide, Para-Aminobenzoic Acid, Pyridoxine hydrochloride, Riboflavin, Thiamine hydrochloride, and Vitamin B12. In several embodiments, the media comprises 1, 2, 3, 4, or more of Calcium nitrate (Ca(NO3)2 4H2O), Magnesium Sulfate (MgSO4) (e.g., Magnesium Sulfate (MgSO4) (anhyd.)), Potassium Chloride (KCl), Sodium Bicarbonate (NaHCO3), Sodium Chloride (NaCl), and Sodium Phosphate dibasic (Na2HPO4) (e.g., Sodium Phosphate dibasic (Na2HPO4) anhydrous).

In several embodiments, the media further comprises D-Glucose and/or glutathione (optionally reduced glutathione). In several embodiments, the media further comprises serum (e.g., fetal bovine serum) in an amount ranging from about 1% to about 20%. In several embodiments, the serum is heat-inactivated. In several embodiments, the media is serum-free. In several embodiments, the media is xenofree.

Depending on the embodiment, IL2 is used to supplement the culture media and enhance expansion, or other characteristics, of NK cells. In several embodiments, the concentration of IL2 used ranges from about 1 IU/mL to about 1000 IU/mL, including for example, about 1 IU/mL to about 5 IU/ml (e.g., 1, 2, 3, 4, and 5, about 5 IU/mL to about 10 IU/mL (e.g., 5, 6, 7, 8, 9, and 10), about 10 IU/mL to about 20 IU/mL (e.g., about 10, 12, 14, 16, 18, and 20), about 20 IU/mL to about 30 IU/mL (e.g., about 20, 22, 24, 26, 28, and 30), about 30 IU/mL to about 40 IU/ml (e.g., 30, 32, 34, 36, 38, and 40), about 40 to about 50 IU/mL (e.g., 40, 42, 44, 46, 48, 50), about 50 IU/mL to about 75 IU/mL (e.g., 50, 55, 60, 65, 70, and 75), about 75 IU/mL to about 100 IU/mL (e.g., 75, 80, 85, 90, 95, and 100), about 100 IU/mL to about 200 IU/mL (e.g., 100, 125, 150, 275, and 200), about 200 IU/mL to about 300 IU/ml (e.g., 200, 225, 250, 275, and 300), about 300 IU/mL to about 400 IU/mL (e.g., 300, 325, 350, 375, and 400), about 400 IU/mL to about 500 IU/ml (e.g., 400, 425, 450, 475, and 500), about 500 IU/mL to about 750 IU/mL (e.g., 500, 550, 600, 650, 700, and 750), or about 750 IU/mL to about 1000 IU/mL (e.g., 750, 800, 850, 900, 950, and 1000), and any concentration therebetween, including endpoints. In several embodiments, IL2 may be added at multiple time points during culture. In some such embodiments the concentration of IL2 used differs between selected time points.

Depending on the embodiment, IL12 (e.g., IL12A and/or IL12B) is used to supplement the culture media and enhance expansion, or other characteristics, of NK cells. In several embodiments, the concentration of IL12 (either IL12A or IL12B) used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/mL to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints. In several embodiments, the concentration of IL12 is between about 0.01 ng/ml and about 8 ng/ml, including any concentration therebetween, including endpoints. In several embodiments, the concentration of IL12 is between about 0.01 ng/ml and about 1 ng/ml, including any concentration therebetween, including endpoints (and including other units of concentration, such as about 0.01 IU/mL to about 1.0 IU/mL, including about 0.5, about 0.6, about 0.7, about 0.8, about 0.9 IU/mL and values in between those listed).

In some embodiments, a mixture of IL12A and IL12B is used. In several embodiments, a particular ratio of IL12A:IL12B is used, for example, 1:10, 1:50, 1:100, 1:150, 1:200, 1:250, 1:500, 1:1000, 1:10,000, 10,000:1, 1000:1, 500:1, 250:1, 150:1, 100:1, 10:1 and any ratio there between, including endpoints.

In some embodiments, interleukin 18 (IL18) is used to enhance expansion, or other characteristics, of NK cells. In several embodiments, the concentration of IL18 used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/mL (e.g., 5.0, 6.0, 7.0, 8.0, 9.0 and 10.0), about 10.0 ng/mL to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/mL to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments interleukin 21 (IL21) is used to enhance expansion, or other characteristics, of NK cells. In several embodiments, the concentration of IL21 used ranges from about 0.01 ng/ml to about 100 ng/ml, including, for example, about 0.01 ng/ml to about 0.05 ng/mL (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/mL (e.g., 0.1, 0.2, 0.3, 0.4, and 0.5), about 0.5 ng/ml to about 1.0 ng/mL (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments interleukin 15 (IL15) is used in a soluble format (either in place of, or in addition to mbIL15 on the feeder cells) to enhance expansion, or other characteristics, of NK cells. In several embodiments, the concentration of IL15 used ranges from about 0.01 ng/ml to about 100 ng/mL, including, for example, about 0.01 ng/ml to about 0.05 ng/ml (e.g., 0.01, 0.02, 0.03, 0.04, and 0.05), about 0.05 ng/mL to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/mL (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/mL to about 5.0 ng/ml (e.g., 2.0, 3.0, 4.0, and 5.0), about 5.0 ng/mL to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/ml to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

In some embodiments interleukin 22 (IL22) is used to facilitate expansion of NK cells. In several embodiments, the concentration of IL22 used ranges from about 0.01 ng/ml to about 100 ng/ml, including, for example, about 0.01 ng/ml to about 0.05 ng/ml (e.g., 0.01, 0.02, 0.03, 0.04, and 0.05), about 0.05 ng/ml to about 0.1 ng/ml (e.g., 0.05, 0.06, 0.07, 0.08, 0.09 and 0.1), about 0.1 ng/ml to about 0.5 ng/ml (e.g., 0.1, 0.2, 0.3, 0.4, and 0.5), about 0.5 ng/ml to about 1.0 ng/ml (e.g., 0.5, 0.6, 0.7, 0.8, 0.9, and 1.0), about 1.0 ng/ml to about 2.0 ng/ml (e.g., 1.1, 1.2, 1.3, 1.4, 1.5, 1.6, 1.7, 1.8, 1.9, and 2.0), about 2.0 ng/ml to about 5.0 ng/ml (e.g., 2.0, 3.0, 4.0, and 5.0), about 5.0 ng/ml to about 10.0 ng/ml (e.g., 5.0, 6.0, 7.0, 8.0, 9.0 and 10.0), about 10.0 ng/ml to about 15.0 ng/ml (e.g., 10.0, 11.0, 12.0, 13.0, 14.0, and 15.0), about 15.0 ng/ml to about 20.0 ng/ml (e.g., 15.0, 16.0, 17.0, 18.0, 19.0, and 20.0), about 20.0 ng/mL to about 25.0 ng/ml (e.g., 20.0, 21.0, 22.0, 23.0, 24.0, and 25.0), about 25.0 ng/ml to about 30.0 ng/ml (e.g., 25.0, 26.0, 27.0, 28.0, 29.0, and 30.0), about 30.0 ng/ml to about 50.0 ng/ml (e.g., 30.0, 35.0, 40.0, 45.0, and 50.0), about 50.0 ng/ml to about 75.0 ng/ml (e.g., 50.0, 55.0, 60.0, 65.0, 70.0, and 75.0), about 75.0 ng/ml to about 100.0 ng/ml (e.g., 75.0, 80.0, 85.0, 90.0, 95.0, and 100.0), and any concentration therebetween, including endpoints.

If two stimulatory agents are used, the relative ratio between the two can range from a ratio of 1:10, 1:20, 1:50, 1:100, 1:150, 1:200, 1:250, 1:500, 1:750, 1:1,000, 1:10,000, 1:50,000, 1:100,000, 100,000:1, 50,000:1, 10,000:1, 1,000:1, 750:1, 500:1, 250:1, 200:1, 150:1, 100:1, 50:1, 20:1, 10:1, and any ratio in between those listed, including endpoints. Likewise, if three, or more, agents are used, the ratio between those additional agents and the other agents can employ any of the aforementioned ratios.

As discussed in more detail below, depending on the embodiment, the stimulatory molecules may be added at a specific point (or points) during the expansion process, or can be added such that they are present as a component of the culture medium through the co-culture process.

### VII. Methods of Co-Culture and Immune Cell Expansion

In some embodiments, NK cells isolated from a donor sample (e.g., peripheral blood) are cultured in the presence of (e.g., co-cultured with) feeder cells. In some embodiments, the NK cells are isolated from a peripheral blood donor sample. In some embodiments, the feeder cells are K562 cells. In some embodiments, the K562 cells express 4-1BBL and a membrane-bound interleukin 15 (mbIL15). In some embodiments, NK cells isolated from a peripheral blood donor sample are co-cultured with K562 cells modified to express 4-1BBL and mbIL15. While other approaches involve the expression of other membrane-bound cytokines, the generation of a feeder cell with multiple stimulatory molecules can be difficult to generate (e.g., to achieve desired levels of expression of the various stimulatory molecule, expression at the right time during expansion, etc.). Thus, several embodiments disclosed herein relate to the supplementation of the culture media with particular concentrations of various stimulatory agents at particular times. In several embodiments, feeder cells are seeded into culture vessels and allowed to reach near confluence. Immune cells can then be added to the culture at a desired concentration, ranging, in several embodiments from about 0.5×10⁶ cells/cm² to about 5×10⁶ cells/cm², including any density between those listed, including endpoints.

In some embodiments, expanding the NK cells comprises culturing the NK cells in the presence of one or more cytokines. In some embodiments, the one or more cytokines comprises interleukin 2 (IL2). In some embodiments, the one or more cytokines comprises interleukin 12 (IL12). In some embodiments, the one or more cytokines comprises interleukin 18 (IL18). In some embodiments, the one or more cytokines comprises any one of IL2, IL12, and/or IL18. In some embodiments, the one or more cytokines comprises IL2 and IL12. In some embodiments, the one or more cytokines comprises IL2 and IL18. In some embodiments, the one or more cytokines comprises IL12 and IL18. In some embodiments, the one or more cytokines comprises IL2, IL12, and IL18.

In some embodiments, expanding the NK cells comprises culturing the NK cells in the presence of feeder cells. Thus, in some embodiments, expanding the NK cells comprises culturing the NK cells in the presence of feeder cells and one or more cytokines. In some embodiments, the NK cells are expanded in culture for between about 7 days and about 84 days. In some embodiments, the NK cells are expanded in culture for about 7 days. In some embodiments, the NK cells are expanded in culture for about 14 days. In some embodiments, the NK cells are expanded in culture for about 21 days. In some embodiments, the NK cells are expanded in culture for about 28 days. In some embodiments, the NK cells are expanded in culture for about 35 days. In some embodiments, the NK cells are expanded in culture for about 42 days. In some embodiments, the NK cells are expanded in culture for about 49 days. In some embodiments, the NK cells are expanded in culture for about 56 days. In some embodiments, the NK cells are expanded in culture for about 63 days. In some embodiments, the NK cells are expanded in culture for about 70 days. In some embodiments, the NK cells are expanded in culture for about 70 days. In some embodiments, the NK cells are expanded in culture for about 84 days.

In several embodiments, immune cells are separated from a peripheral blood sample. In some embodiments, the immune cells comprise NK cells. In some embodiments, the immune cells are NK cells. Thereafter, in several embodiments, the immune cells can be expanded together, or an isolated subpopulation of cells, such as NK cells, is used.

Thereafter, the NK cells are seeded with the feeder cells, and optionally one or more cytokines (either in the culture media or as an exogenous supplement) and cultured for a first period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. Each exposure (e.g., co-culture) to fresh feeder cells is referred to herein as a pulse.

In several embodiments, during the expansion process, for example, after the first period of expansion, the expanded cells (e.g., NK cells) are transduced with an engineered construct, such as a chimeric antigen receptor. Any variety of chimeric antigen receptor can be expressed in the engineered cells, such as NK cells, including those described in International PCT Application PCT/US2018/024650, PCT/IB2019/000141, PCT/IB2019/000181, and/or PCT/US2020/020824, PCT/US2020035752, PCT/US2021/036879, or U.S. Provisional Application No. 63/220,842, each of which is incorporated in its entirety by reference herein.

In several embodiments, the expanding cells are pulsed again with fresh feeder cells and cultured for about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. The cells can then optionally be separated into multiple aliquots and stored (e.g., cryopreserved as a master cell bank) from which future expansions can be performed. In several embodiments, generation of a master cell bank involves 1 to 3 or 1 to 4 pulses with feeder cells and co-culturing for a total time ranging from about 14 days to about 36 days.

In several embodiments, cells that have been expanded and engineered (and optionally gene edited) are pulsed at least one additional time and are cultured for a period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, or for any time between those listed, including endpoints. It shall be noted that certain data presented herein relates to viral expression of a chimeric receptor complex expressing an NKG2D ligand binding domain (e.g., NKX101) or CD19 (e.g., NK19-1 or NKX019). However, any suitable chimeric receptor or chimeric antigen receptor can be used. Cells may optionally be separated into additional aliquots and cryopreserved (e.g., as a working cell bank) from which further expansion can be performed. In several embodiments, generation of a working cell bank involves 1 to 3 or 1 to 4 pulses with feeder cells and co-culturing for a total time ranging from about 14 days to about 36 days.

In several embodiments, cells that have been expanded to the working cell bank are subjected to at least one additional pulse of feeder cells and are cultured for a period of time, for example about 6 hours, about 12 hours, about 18 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 18 days or about 21 days, or for any time between those listed, including endpoints. In several embodiments, at the termination of this co-culture, the cells have been sufficiently expanded and are aliquoted into individual patient doses and stored (e.g., cryopreserved) until administration.

In some embodiments, expanding the NK cells comprises culturing the NK cells for a first time in the presence of feeder cells and one or more cytokines. In some embodiments, culturing the NK cells for the first time is between about 5 days and about 20 days, or between about 10 days and about 15 days. In some embodiments, culturing the NK cells for the first time is between about 5 days and about 20 days. In some embodiments, culturing the NK cells for the first time is between about 10 days and about 15 days. In some embodiments, expanding the NK cells comprises culturing the NK cells for a second time in the presence of feeder cells and one or more cytokines. In some embodiments, culturing the NK cells for the second time is between about 5 days and about 20 days, or between about 10 days and about 15 days. In some embodiments, culturing the NK cells for the second time is between about 5 days and about 20 days. In some embodiments, culturing the NK cells for the second time is between about 10 days and about 15 days. Thus, in some embodiments, expanding the NK cells comprises (a) culturing the NK cells in the presence of feeder cells and one or more cytokines for a first time of between about 10 days and about 15 days; and (b) culturing the NK cells in the presence of feeder cells and one or more cytokines for a second time of between about 10 days and about 15 days. In some embodiments, the NK cells are expanded in culture for between about 20 days and about 30 days.

Supplementation of the media with one or more stimulatory agents (e.g., cytokines), such as IL12 and/or IL18 can occur at any time during the culturing process. For example, one or more stimulatory agents can be added at the inception of culturing, for example at time point zero (e.g., inception of culture). The agent, or agents, can be added a second, third, fourth, fifth, or more times. Subsequent additions may, or may not, be at the same concentration as a prior addition. The interval between multiple additions can vary, for example a time interval of about 12 hours, about 24 hours, about 36 hours, about 48 hours, about 72 hours, or longer, and any time therebetween, including endpoints.

If multiple additions of a stimulatory agent (e.g., cytokine) are used, the concentrations of a first supplemental addition can be at the same or a different concentration than the second (and/or any supplemental addition). For example, in several embodiments, the addition of a stimulatory agent over multiple time points can ramp up, ramp down, stay constant, or vary across multiple, non-equivalent concentrations.

In several embodiments, certain ratios of feeder cells to cells to be expanded are used. For example, in several embodiments a feeder cell: “target” cell ratio of about 10:1 to about 2:1 is used, including, for example 9:1, 8:1, 7:1, 6:1, 5:1, 4:1, 3:1 and any ratio therebetween, including endpoints. In several embodiments the feeder cell to NK cell ratio is about 10:1. In several embodiments the feeder cell to NK cell ratio is about 9:1. In several embodiments the feeder cell to NK cell ratio is about 8:1. In several embodiments the feeder cell to NK cell ratio is about 7:1. In several embodiments the feeder cell to NK cell ratio is about 6:1. In several embodiments the feeder cell to NK cell ratio is about 5:1. In several embodiments the feeder cell to NK cell ratio is about 4:1. In several embodiments the feeder cell to NK cell ratio is about 3:1. In several embodiments the feeder cell to NK cell ratio is about 2:1.

In several embodiments, a 1:1 ratio is used, while in additional embodiments, can range from about: 1:10, 1:20, 1:50, 1:100, 1:1,000, 1:10,000, 1:50,000, 1:100,000, 100,000:1, 50,000:1, 10,000:1, 1,000:1, 100:1, 50:1, 20:1, 10:1, and any ratio in between those listed, including endpoints. In several embodiments, different feeder:target ratios are used at different pulses. In several embodiments, the degree of expansion is such that the resulting population is expanded by at least about 1000-fold, about 5000-fold, about 10,000-fold, about 50,000-fold, about 100,000-fold, about 500,000-fold, about 1 million-fold, about 2 million-fold, about 5 million-fold, about 20 million-fold, about 50 million-fold, about 100 million-fold, about 200 million-fold, about 500 million fold, about 800 million-fold, about 1 billion-fold, about 2 billion-fold or more (or any amount between those listed).

### VIII. Compositions and Formulations

Also provided are compositions including NK cells produced by any of the methods described herein, including pharmaceutical compositions and formulations. Provided are pharmaceutical formulations comprising NK cells produced by any of the methods described herein and/or additional agents for combination treatment or therapy. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell, binding molecule, and/or antibody, and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some aspects, a buffer is included in the composition. Suitable buffering agents include, for example, citric acid, sodium citrate, phosphoric acid, potassium phosphate, and various other acids and salts. In some aspects, a mixture of two or more buffers is used. The buffering agent or mixtures thereof are typically present in an amount of from about 0.001% to about 4% by weight of the total composition. Methods for preparing administrable pharmaceutical compositions are known.

Formulations of the antibodies described herein can include lyophilized formulations and aqueous solutions. The formulation or composition may also contain more than one active ingredient useful for the particular indication, disease, or condition being treated with the cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., methotrexate or rituximab. In some embodiments, the cells or antibodies are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains the NK cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the agent or cell population is administered to the subject by intravenous, intraperitoneal, or subcutaneous injection using peripheral systemic delivery.

In some embodiments, the compositions are provided as sterile liquid formulations (e.g., isotonic aqueous solutions, suspensions, emulsions, dispersions, or viscous compositions), which in some aspects may be buffered to a selected pH. Liquid formulations are generally easier to prepare than gels, other viscous compositions, and solid compositions. In addition, liquid compositions are somewhat more convenient to administer, particularly by injection. The liquid composition can comprise a carrier, which can be a solvent or dispersion medium containing, for example, water, saline, phosphate buffered saline, polyols (e.g., glycerol, propylene glycol, liquid polyethylene glycol), and suitable mixtures thereof.

Sterile injectable solutions can be prepared by incorporating the agent or cell into a solvent, such as an admixture with a suitable carrier, diluent, or excipient (e.g., sterile water, saline, glucose, dextrose, and the like). Formulations for in vivo administration are typically sterile. Sterility can be readily achieved, for example, by filtration through sterile filtration membranes. In some embodiments, the dose of engineered cells administered is in a cryopreserved composition. In some aspects, the composition is administered after thawing the cryopreserved composition.

### IX. Articles of Manufacture and Kits

Also provided are articles of manufacture or kits containing the provided NK cells and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Non-limiting examples of containers include intravenous solution bags and vials, including those with stoppers pierceable by a needle for injection. The article of manufacture or kit may further include a package insert indicating that the composition can be used to treat a particular condition such as a disease or condition described herein (e.g., a cancer, tumor, or autoimmune disease). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating a disease or condition (e.g., a cancer, tumor, or autoimmune disease) in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a disease or condition (e.g., a cancer, tumor, or autoimmune disease) in an individual.

The container in some embodiments holds a composition which is by itself or combined with another composition effective for treating, preventing and/or diagnosing the disease or condition (e.g., a cancer, tumor, or autoimmune disease). The article of manufacture or kit may include (a) a first container with a composition contained therein (i.e., first medicament), wherein the composition includes the NK cells; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### X. Methods of Treatment and Uses

Provided herein are methods of treatment comprising administering any of the populations of NK cells described herein or a composition containing a population of NK cells to a subject (e.g., a subject having a disease or condition). In some aspects, there is also provided a use of any of the populations of NK cells described herein or a composition containing a the population of NK cells for treating a disease or condition. In some aspects, there is also provided a use of any of the populations of NK cells described herein or a composition containing the population of NK cells for the manufacture of a medicament to treat a disease or condition.

Diseases and disorders include cancers and tumors, including solid tumors, hematologic malignancies and melanoma, and include local and metastatic tumors; infectious diseases, such as infection by a virus or other pathogen, e.g., HIV, HCV, HBV, CMV, HPV and parasitic diseases; and autoimmune and inflammatory diseases.

In some embodiments, the disease or disorder is an infectious disease or disorder, such as, but not limited to, viral, retroviral, bacterial and protozoal infections, immunodeficiency, Cytomegalovirus (CMV), epstein-barr virus (EBV), adenovirus, and BK polyomavirus.

In some embodiments, the disease or disorder is an autoimmune or inflammatory disease or disorder, such as arthritis (e.g., Rheumatoid Arthritis (RA)), type I diabetes, Systemic Lupus Erythematosus (SLE), lupus nephritis (LN), inflammatory bowel disease (IBD), psoriasis, scleroderma (e.g., systemic sclerosis), autoimmune thyroid disease, Graves' disease, Crohn's disease, multiple sclerosis (MS), myasthenia gravis (MG), myositis, antisynthetase syndrome, asthma, and/or a disease or disorder associated with transplantation. In some embodiments, the disease or disorder is an autoimmune disease. In some embodiments, the disease or disorder is a B cell-mediated autoimmune disease. In some embodiments, the disease or disorder is a T cell-mediated autoimmune disease. In some embodiments, the disease or disorder is a plasma cell-mediated autoimmune disease. In some embodiments, the autoimmune disease is RA. In some embodiments, the autoimmune disease is type I diabetes. In some embodiments, the autoimmune disease is SLE. In some embodiments, the autoimmune disease is LN. In some embodiments, the autoimmune disease is IBD. In some embodiments, the autoimmune disease is psoriasis. In some embodiments, the autoimmune disease is scleroderma (e.g., systemic sclerosis). In some embodiments, the autoimmune disease is autoimmune thyroid disease. In some embodiments, the autoimmune disease is Graves' disease. In some embodiments, the autoimmune disease is Crohn's disease. In some embodiments, the autoimmune disease is MS. In some embodiments, the autoimmune disease is MG. In some embodiments, the autoimmune disease is myositis. In some embodiments, the autoimmune disease is antisynthetase syndrome. In some embodiments, the autoimmune disease is asthma.

In some embodiments, the disease or disorder is a tumor, cancer, malignancy, neoplasm, or other proliferative disease or disorder. In some embodiments, the disease or disorder is cancer. Examples of cancer include, but are not limited to, acute lymphoblastic leukemia (ALL), acute myeloid leukemia (AML), adrenocortical carcinoma, Kaposi sarcoma, lymphoma, gastrointestinal cancer, appendix cancer, central nervous system cancer, basal cell carcinoma, bile duct cancer, bladder cancer, bone cancer, brain tumors (including but not limited to astrocytomas, spinal cord tumors, brain stem glioma, craniopharyngioma, ependymoblastoma, ependymoma, medulloblastoma, medulloepithelioma), breast cancer, bronchial tumors, Burkitt lymphoma, cervical cancer, colon cancer, chronic lymphocytic leukemia (CLL), chronic myelogenous leukemia (CML), chronic myeloproliferative disorders, ductal carcinoma, endometrial cancer, esophageal cancer, gastric cancer, Hodgkin lymphoma, non-Hodgkin lymphoma, hairy cell leukemia, renal cell cancer, leukemia, oral cancer, nasopharyngeal cancer, liver cancer, lung cancer (including but not limited to, non-small cell lung cancer, (NSCLC) and small cell lung cancer), pancreatic cancer, bowel cancer, lymphoma, melanoma, ocular cancer, ovarian cancer, pancreatic cancer, prostate cancer, pituitary cancer, uterine cancer, and vaginal cancer.

In some embodiments, the cancer comprises a tumor. In some embodiments, the cancer comprises a solid tumor. In some embodiments, the cancer is a hematologic malignancy.

In some embodiments, the cancer is multiple myeloma. In some embodiments, the cancer is a leukemia or a lymphoma. In some embodiments, the cancer is a leukemia. In some embodiments, the leukemia is ALL, AML, CLL, or CML. In some embodiments, the cancer is a lymphoma. In some embodiments, the lymphoma is Hodgkin lymphoma or non-Hodgkin lymphoma (NHL). In some embodiments, the cancer is NHL. In some embodiments, the NHL is large B-cell lymphoma (LBCL). In some embodiments, the NHL is aggressive NHL. In some embodiments the NHL is a diffuse large B-cell lymphoma (DLBCL), Burkitt lymphoma, follicular lymphoma (FL, including grade 1, 2, 3a, and 3b), small lymphocytic lymphoma (SLL), mantle cell lymphoma, or marginal zone lymphoma. In some embodiments, the NHL is DLBCL. In some embodiments, the NHL is FL. In some embodiments, the NHL is mantle cell lymphoma. In some embodiments, the NHL is marginal zone lymphoma.

In some embodiments, the cancer is relapsed/refractory. In some embodiments, the cancer is relapsed refractory to a prior line of therapy. In some embodiments, the prior line of therapy comprises one prior line of therapy. In some embodiments, the prior line of therapy comprises two prior lines of therapy. In some embodiments, the prior line of therapy comprises three prior lines of therapy.

Some embodiments relate to a method of treating, ameliorating, inhibiting, or preventing cancer with a population of NK cells as described herein. In some embodiments, the method includes treating or preventing a tumor or cancer. Some embodiments relate to a method of treating, ameliorating, inhibiting, or preventing an autoimmune disease with a population of NK cells as described herein. In some embodiments, the method includes treating or preventing an autoimmune disease. In some embodiments, the method includes administering a therapeutically effective amount of a population of NK cells as described herein.

In certain embodiments, treatment of a subject with a population of NK cells described herein achieves one, two, three, four, or more of the following effects, including, for example: (i) reduction or amelioration the severity of disease or symptom associated therewith; (ii) reduction in the duration of a symptom associated with a disease; (iii) protection against the progression of a disease or symptom associated therewith; (iv) regression of a disease or symptom associated therewith; (v) protection against the development or onset of a symptom associated with a disease; (vi) protection against the recurrence of a symptom associated with a disease; (vii) reduction in the hospitalization of a subject; (viii) reduction in the hospitalization length; (ix) an increase in the survival of a subject with a disease; (x) a reduction in the number of symptoms associated with a disease; and (xi) an enhancement, improvement, supplementation, complementation, or augmentation of the prophylactic or therapeutic effect(s) of another therapy.

Doses of immune cells such as NK cells can be readily determined for a given subject based on their body mass, disease type and state, and desired aggressiveness of treatment, but range, depending on the embodiments, from about 10⁵ cells per kg to about 10¹² cells per kg (e.g., 10⁵-10⁷, 10⁷-10¹⁰, 10¹⁰-10¹² and overlapping ranges therein). In one embodiment, a dose escalation regimen is used. In several embodiments, a range of NK cells is administered, for example between about 1×10⁶ cells/kg to about 1×10⁸ cells/kg. Depending on the embodiment, various types of diseases or conditions can be treated. For the prevention or treatment of cancer, the appropriate dosage may depend on the type of cancer to be treated, the type of cells or recombinant receptors, the severity and course of the cancer, whether the cells are administered for preventive or therapeutic purposes, previous therapy, the subject's clinical history and response to the cells, and the discretion of the attending physician. For the prevention or treatment of an autoimmune disease, the appropriate dosage may depend on the type of autoimmune disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the method comprises administration of between about 100×10⁶ NK cells and about 10×10⁹ NK cells, or between about 300×10⁶ NK cells and about 1.5×10⁹ NK cells to a subject. In some embodiments, the method comprises administration of between about 100×10⁶ NK cells and about 10×10⁹ NK cells to a subject. In some embodiments, the method comprises administration of between about 300×10⁶ NK cells and about 1.5×NK cells to a subject. In some embodiments, the method comprises administration of about 300×10⁶ NK cells to a subject.

In some embodiments, the method comprises administration of about 1×10⁹ NK cells to a subject. In some embodiments, the method comprises administration of about 1.5×10⁹ NK cells to a subject. In some embodiments, the method comprises administration of about 2×10⁹ NK cells to a subject. In some embodiments, the method comprises administration of about 2.5×10⁹ NK cells to a subject. In some embodiments, the method comprises administration of about 3×10⁹ NK cells to a subject. In some embodiments, the method comprises administration of about 4×10⁹ NK cells to a subject. In some embodiments, the method comprises administration of about 5×10⁹ NK cells to a subject.

Further provided herein are methods of treating a subject having cancer, comprising administering to the subject a composition comprising a population of NK cells, for example NK cells engineered to express a chimeric receptor complex as disclosed herein. In some aspects, the compositions and methods described herein relate to use of a tumor-directed chimeric receptor, or use of a population of NK cells expressing a tumor-directed chimeric receptor, for treating a cancer patient. Uses of such populations of NK cells for treating cancer are also provided.

In some embodiments, the NK cells are autologous to a subject to be treated. In some embodiments, the NK cells are obtained from a subject (e.g., a first subject) other than the subject that will receive or ultimately receives the NK cells. In such embodiments, the NK cells are then administered to a different subject of the same species, e.g., a second subject. In some embodiments, the first and second subjects are genetically similar. In some embodiments, the second subject expresses the same HLA class or supertype as the first subject. In some embodiments, the NK cells are allogeneic to a subject to be treatment.

In some embodiments, the population of NK cells is administered as part of a combination treatment, such as simultaneously with or sequentially with, in any order, another or additional therapeutic intervention, such as an antibody or engineered cell or receptor or agent, such as a cytotoxic or therapeutic agent. The population of NK cells in some embodiments is co-administered with one or more additional therapeutic agents or in connection with another therapeutic intervention, either simultaneously or sequentially in any order. In some embodiments, the additional therapeutic agent is any interventions or agents described herein, such as any interventions or agents described that can ameliorate symptoms of toxicity. In some contexts, the population of NK cells is co-administered with another therapy sufficiently close in time such that the engineered NK cell populations enhance the effect of one or more additional therapeutic agents, or vice versa. In some embodiments, the population of NK cells is administered prior to the one or more additional therapeutic agents. In some embodiments, the population of NK cells is administered after the one or more additional therapeutic agents. In some embodiments, the methods comprise administration of a chemotherapeutic agent. In some embodiments, the methods comprise administration of a chemotherapeutic agent, e.g., a conditioning chemotherapeutic agent, for example, to reduce tumor burden prior to the administration, including as described below.

The dose of the additional agent can be any therapeutically effective amount, e.g., any dose amount described herein, and the appropriate dosage of the additional agent may depend on the type of disease to be treated, the type, dose and/or frequency of the binding molecule, recombinant receptor, cell and/or composition administered, the severity and course of the disease, previous therapy, the patient's clinical history and response to cell therapy, and the discretion of the attending physician.

In some embodiments, prior to treatment of a subject, the subject has been administered a lymphodepleting therapy. Preconditioning subjects with immunodepleting (e.g., lymphodepleting) therapies in some aspects can improve the effects of a cell therapy, including any of those provided herein. Thus, in some embodiments, the methods include administering a preconditioning agent, such as a lymphodepleting or chemotherapeutic agent to a subject prior to administration of the population of NK cells. For example, the subject may be administered a preconditioning agent at least 2 days prior, such as at least 3, 4, 5, 6, or 7 days prior, to administration of the population of NK cells. In some embodiments, the subject is administered a preconditioning agent no more than 7 days prior, such as no more than 6, 5, 4, 3, or 2 days prior, to administration of the population of NK cells. In some embodiments, the lymphodepleting agent comprises cyclophosphamide. In some embodiments, the lymphodepleting agent comprises cyclophosphamide and fludarabine. In some embodiments, the lymphodepleting agent comprises fludarabine. In some embodiments, the lymphodepleting agent comprises fludarabine and cytarabine (Ara-C).

### NON-LIMITING EMBODIMENTS

Among the embodiments provided herein are:
1. A method of enriching for educated natural killer (NK) cells from a biological sample, comprising, from a biological sample comprising NK cells, enriching for cells that express an inhibitory Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+),
the enriching thereby generating an enriched population comprising educated NK cells.
2. A method of increasing the expansion of natural killer cells, the method comprising:
from a biological sample comprising NK cells, enriching for cells that express an inhibitor Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), the enriching thereby generating an enriched population comprising educated NK cells; and
expanding the enriched population comprising educated NK cells, thereby generating an expanded NK cell population.
3. The method of embodiment 1 or embodiment 2, comprising enriching for cells that both (i) express an iKIR selected from among the group consisting of: KIR2DL1, KIR2DL2, KIR2DL3, KIR3DL1, KIR3DL2, KIR3DL3, KIR2DL5, and any combination thereof (iKIR+); and (ii) do not express NKG2A (NKG2A−).
4. The method of any one of embodiments 1-3, wherein at least about 25%, at least about 30%, at least about 35%, at least about 40%, at least about 45%, or at least about 50% of cells in the enriched population comprise NK cells that express an iKIR (iKIR+ cells).
5. The method of any one of embodiments 1-4, wherein at least about 10%, at least about 15%, at least about 20%, at least about 25%, or at least about 30% of cells in the enriched population comprise NK cells that both express an iKIR and do not express NKG2A (iKIR+NKG2A− cells).
6. The method of any one of embodiments 1-5, comprising obtaining the biological sample from a donor.
7. The method of any one of embodiments 1-6, wherein the biological sample is a blood sample.
8. The method of embodiment 7, wherein the blood sample is a peripheral blood sample.
9. The method of embodiment 7, wherein the blood sample is a cord blood sample.
10. The method of any one of embodiments 1-9, wherein enriching for cells that express an iKIR comprises enriching for cells that express one iKIR, two iKIRs, or three iKIRs.
11. The method of any one of embodiments 1-10, wherein enriching for cells that express an iKIR comprises positive selection.
12. The method of any one of embodiments 3-11, wherein enriching for cells that do not express NKG2A comprises negative selection.
13. The method of any one of embodiments 1-12, wherein enriching for cells that express an iKIR comprises contacting the NK cells with an antibody that binds to iKIR.
14. The method of any one of embodiments 3-13, wherein enriching for cells that do not express NKG2A comprises contacting the NK cells with an antibody that binds to NKG2A.
15. The method of any one of embodiments 1 and 3-14, comprising expanding the enriched population comprising educated NK cells, thereby generating an expanded NK cell population.
16. The method of embodiment 15, where expanding the enriched population comprising educated NK cells comprises co-culturing NK cells of the enriched population with a feeder cell population, optionally in a culture media supplemented with soluble interleukin 12 (IL12) and/or soluble interleukin 18 (IL18).
17. The method of embodiment 16, wherein the feeder cell population comprises cells engineered to express 4-1BBL and membrane-bound interleukin-15 (mbIL15).
18. The method of embodiment 16 or embodiment 17, wherein the ratio of NK cells to feeder cells ranges from about 1:2 to about 1:10, optionally from about 1:3 to about 1:5.
19. The method of any one of embodiments 16-18, wherein: (a) the IL12 is present in the supplemented culture media at a concentration ranging from about 0.01 ng/ml to about 10 ng/ml; and/or (b) the IL18 is present in the supplemented culture media at a concentration ranging from about 10 ng/ml to about 30 ng/ml.
20. The method of any one of embodiments 16-19, wherein the culture media is supplemented with soluble interleukin 2 (IL2).
21. The method of embodiment 20, wherein the IL2 is present in the supplemented media at a concentration ranging from about 25 units/mL to about 50 units/mL.
22. The method of any of embodiments 2-21, wherein expanding the enriched population comprising educated NK cells does not comprise contacting NK cells with a stimulator of NKG2A, NKG2C, or both.
23. The method of any of embodiments 2-22, wherein expanding the enriched population comprising educated NK cells does not comprise contacting NK cells with HLA-E.
24. The method of any of embodiments 16-23, wherein the feeder cells of the feeder cell population do not express HLA-E.
25. The method of any one of embodiments 2-24, wherein expanding the enriched population comprising educated NK cells comprises co-culturing NK cells of the enriched population with a feeder cell population for a first time, optionally wherein expanding the enriched population comprising educated NK cells comprises co-culturing NK cells of the enriched population with a feeder cell population for a second time.
26. The method of any one of embodiments 2-25, wherein: (a) the method comprises genetically editing NK cells of the expanded NK cell population or the biological sample to reduce or eliminate expression of at least one endogenous gene or protein expressed as compared to a non-modified NK cell, thereby generating a genetically edited NK cell population; and/or (b) NK cells of the expanded NK cell population or the biological sample are genetically edited to reduce or eliminate expression of at least one endogenous gene or protein expressed as compared to a non-modified NK cell, thereby generating a genetically edited NK cell population.
27. The method of any one of embodiments 2-26, wherein: (a) the method comprises engineering NK cells of the expanded NK cell population or the genetically edited NK cell population to express a chimeric antigen receptor (CAR) that binds an antigen, thereby generating an engineered NK cell population; and/or (b) NK cells of the expanded NK cell population or the genetically edited NK cell population are engineered to express a chimeric antigen receptor (CAR) that binds to an antigen, thereby generating an engineered NK cell population.
28. The method of embodiment 27, wherein the antigen is selected from a ligand for the NKG2D receptor, CD19, CD70, BCMA, or CD38.
29. The method of embodiment 27 or embodiment 28, wherein the CAR comprises a transmembrane domain and an intracellular signaling region.
30. The method of embodiment 29, wherein the transmembrane domain comprises CD8, CD28, or a portion thereof, optionally wherein the transmembrane domain comprises CD8a or a portion thereof.
31. The method of embodiment 29 or embodiment 30, wherein the intracellular signaling region comprises a CD3zeta domain.
32. The method of any one of embodiments 29-31, wherein the intracellular signaling region comprises an intracellular signaling domain of an OX40, 4-1BB, CD28, or a signaling portion thereof, optionally wherein the intracellular signaling region comprises an intracellular signaling domain of OX40 or a signaling portion thereof.
33. The method of any one of embodiments 2-32, wherein: (a) the method comprises engineering NK cells of the expanded NK cell population, the genetically edited NK cell population, or the engineered NK cell population to express membrane-bound interleukin 15 (mbIL15); and/or (b) NK cells of the expanded NK cell population, the genetically edited NK cell population, or the engineered NK cell population are engineered to express membrane-bound interleukin 15 (mbIL15).
34. A method of producing a population of engineered natural killer (NK) cells, the method comprising:
(a) from a biological sample comprising NK cells, enriching for cells that express an inhibitor Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), the enriching thereby generating an enriched population comprising educated NK cells;
(b) expanding the enriched population comprising educated NK cells, thereby generating an expanded NK cell population; and
(c) engineering NK cells of the expanded NK cell population to express a chimeric antigen receptor (CAR) that binds an antigen, thereby generating an engineered NK cell population.
35. A population of NK cells produced by the method of any one of embodiments 1-34.
36. The population of NK cells of embodiment 35 for use in the treatment of a disease or condition in a subject.
37. The population of NK cells of embodiment 35, wherein the disease or condition is a cancer.
38. The population of NK cells of embodiment 35, wherein the disease or condition is an autoimmune disease.
39. Use of the population of NK cells of embodiment 34 for the treatment of a disease or condition in a subject.
40. Use of the population of NK cells of embodiment 34 in the preparation of a medicament for the treatment of a disease or condition in a subject.
41. A method of treating a disease or condition in a subject, comprising administering to the subject the population of NK cells of embodiment 34.
42. The population of NK cells for use of any one of embodiments 35-37, the use of embodiment 38 or embodiment 39, or the method of embodiment 40, wherein the population of NK cells is allogeneic to the subject.
43. The population of NK cells for use of embodiment 36 or embodiment 42, the use of any one of embodiments 39, 40, and 42, or the method of embodiment 41 or embodiment 42, wherein the disease or condition is a cancer.
44. The population of NK cells for use of embodiment 37 or embodiment 43, the use of embodiment 42, or the method of embodiment 42, wherein the cancer is a hematologic malignancy, optionally wherein the cancer is a leukemia or a lymphoma.
45. The population of NK cells for use of any one of embodiments 37, 43, and 44, the use of embodiment 42 or embodiment 44, or the method of embodiment 43 or embodiment 44, wherein the CAR binds to an antigen expressed by the cancer.
46. The population of NK cells for use of embodiment 36 or embodiment 42, the use of any one of embodiments 39, 40, and 42, or the method of embodiment 41 or embodiment 42, wherein the disease or condition is an autoimmune disease.
47. The method of any one of embodiments 1-46, wherein the biological sample is from a subject who is CMV positive (CMV+).
48. The method of any one of embodiments 1-47, wherein the iKIR+ cells do not express CD57 (CD57−).
49. The method of any one of embodiments 3-48, wherein the iKIR+NKG2A− cells do not express CD57 (CD57−).
50. The method of any one of embodiments 1-49, wherein the iKIR+ cells do not express NKG2C (NKG2C−).
51. The method of any one of embodiments 3-50, wherein the iKIR+NKG2A− cells do not express NKG2C (NKG2C−).
52. A method for selecting a donor of natural killer (NK) cells for immunotherapy, comprising
(a) assessing in a biological sample from a donor, the percentage of cells expressing an inhibitory Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), wherein the biological sample comprises NK cells;
(b) determining whether the percentage of iKIR+ cells in the biological sample is at or above a threshold value, wherein the threshold value is at least about 25%; and
(c) if the percentage of iKIR+ cells is at or above the threshold value, selecting the biological sample for use in immunotherapy; or
   if the percentage of iKIR+ cells is less than the threshold value, not selecting the biological sample for use in immunotherapy.
53. The method of embodiment 52, wherein the threshold value is about 25%, about 30%, about 35%, about 40%, about 45%, or about 50%.
54. A method for selecting a donor of natural killer (NK) cells for immunotherapy, comprising
(a) assessing in a biological sample from a donor, the percentage of cells that both (i) express an inhibitory Killer Cell Ig-like Receptor (iKIR) selected from among the group consisting of: KIR2DL1, KIR2DL2, KIR2DL3, KIR3DL1, KIR3DL2, KIR3DL3, KIR2DL5, or any combination thereof (iKIR+); and (ii) do not express NKG2A (NKG2A−), wherein the biological sample comprises NK cells;
(b) determining whether the percentage of iKIR+NKG2A− cells in the biological sample is at or above a threshold value, wherein the threshold value is at least about 10%; and
(c) if the percentage of iKIR+NKG2A− cells is at or above the threshold value, selecting the biological sample for use in immunotherapy; or
   if the percentage of iKIR+NKG2A− cells is less than the threshold value, not selecting the biological sample for use in immunotherapy.
55. The method of embodiment 54, wherein the threshold value is about 10%, about 15%, about 20%, about 25%, or about 30%.
56. The method of any one of embodiments 52-55, comprising obtaining the biological sample from the donor.
57. The method of any one of embodiments 52-56, wherein the biological sample is a blood sample.
58. The method of embodiment 57, wherein the blood sample is a peripheral blood sample.
59. The method of embodiment 57, wherein the blood sample is a cord blood sample.
60. The method of any one of embodiments 52-59, wherein cells that express an iKIR express one iKIR, two iKIRs, or three iKIRs.
61. The method of any one of embodiments 52-60, wherein if the biological sample is selected for use in immunotherapy, the method comprises expanding the NK cells of the biological sample, thereby generating an expanded NK cell population.
62. The method of embodiment 61, wherein expanding the NK cells of the biological sample comprises co-culturing, in a culture media supplemented with soluble interleukin 12 (IL12) and soluble interleukin 18 (IL18), NK cells of biological sample with a feeder cell population.
63. The method of embodiment 62, wherein the feeder cell population comprises cells engineered to express 4-1BBL and membrane-bound interleukin-15 (mbIL15).
64. The method of any one of embodiments 61-63, wherein expanding the NK cells of the biological sample does not comprise contacting NK cells with a stimulator of NKG2A, NKG2C, or both.
65. The method of any one of embodiments 61-64, wherein expanding the NK cells of the biological sample does not comprise contacting NK cells with HLA-E.
66. The method of any one of embodiments 62-65, wherein the feeder cells of the feeder cell population do not express HLA-E.
67. The method of any one of embodiments 52-66, comprising genetically editing NK cells of the expanded NK cell population or the biological sample to reduce or eliminate expression of at least one endogenous gene or protein expressed as compared to a non-modified NK cell, thereby generating a genetically edited NK cell population.
68. The method of embodiment 67, wherein the genetic editing comprises a disruption of the cytokine-inducible SH2-containing protein (CISH) gene, thereby resulting in reduced or eliminated CIS expression by the NK cell.
69. The method of any one of embodiments 61-68, comprising engineering NK cells of the expanded NK cell population or the genetically edited NK cell population to express a chimeric antigen receptor (CAR) that binds a tumor antigen, thereby generating an engineered NK cell population.
70. The method of any one of embodiments 52-69, wherein the immunotherapy is for treating a disease or condition in a subject.
71. The method of embodiment 70, wherein the disease or condition is a cancer.
72. The method of embodiment 71, wherein the cancer is a hematologic malignancy, optionally wherein the cancer is a leukemia or a lymphoma.
73. The method of embodiment 71 or embodiment 72, wherein the CAR binds to an antigen expressed by the cancer.
74. The method of embodiment 70, wherein the disease or condition is an autoimmune disease.
75. The method of any one of embodiments 70-74, wherein the NK cells of the biological sample are allogeneic to the subject.
76. The method of any one of embodiments 52-75, wherein the iKIR+ cells do not express CD57 (CD57−).
77. The method of any one of embodiments 54-76, wherein the iKIR+NKG2A− cells do not express CD57 (CD57−).
78. The method of any one of embodiments 52-77, wherein the iKIR+ cells do not express NKG2C (NKG2C−).
79. The method of any one of embodiments 54-78, wherein the iKIR+NKG2A− cells do not express NKG2C (NKG2C−).
80. The method of any one of embodiments 52-79, wherein the donor is CMV positive (CMV+).

### EXAMPLES

The materials and methods disclosed in the Examples are non-limiting examples of materials and methods (including reagents and conditions) applicable to various embodiments provided in the present application.

### Example 1—Donor Expansion and Cytotoxicity Ranking

As discussed above, in several embodiments, a candidate donor is screened for cells that exhibit qualities that render the donor a preferred donor, whether that be potential for expansion or potentially enhanced cytotoxicity. In this non-limiting example, twelve donors were screened for their KIR profiles, as discussed above, and their expansion capacity and cytotoxicity after being expanded according to the expansion methods disclosed herein. As a non-limiting example, these donor NK cells were engineered to express an anti-CD19 CAR construct, for which additional information can be found in International Patent Application No. PCT/US2020/020824, the entire contents of which is incorporated by reference herein.

FIG. 1B shows data related to the expansion profile of NK cells from twelve donors after engineering to express an anti-CD19 CAR (or untransduced control) and expanded using the IL12/IL18 multiple pulse expansion methods disclosed herein, or without IL12/18. FIG. 1C shows corresponding cytotoxicity data. As can be seen in FIG. 1B, the presence or absence of IL12/IL18 in the culture process did not significantly impact expansion of the NK cells (whether expressing a CAR or not). Firstly, as shown in FIG. 1C, the transduction of the NK cells with the anti-CD19 CAR enhances the cytotoxicity profile of the NK cells, and notably, the use of IL12/IL18 in the expansion process further, and significantly, increased the cytotoxicity of the NK cells (against NALM6 tumor cells at a 1:4 E:T ratio at 96 hrs post-transduction. This data suggests that donor cells can obtain enhanced characteristics through the culturing process and based on their ability to respond to stimulatory cytokines in the culture process.

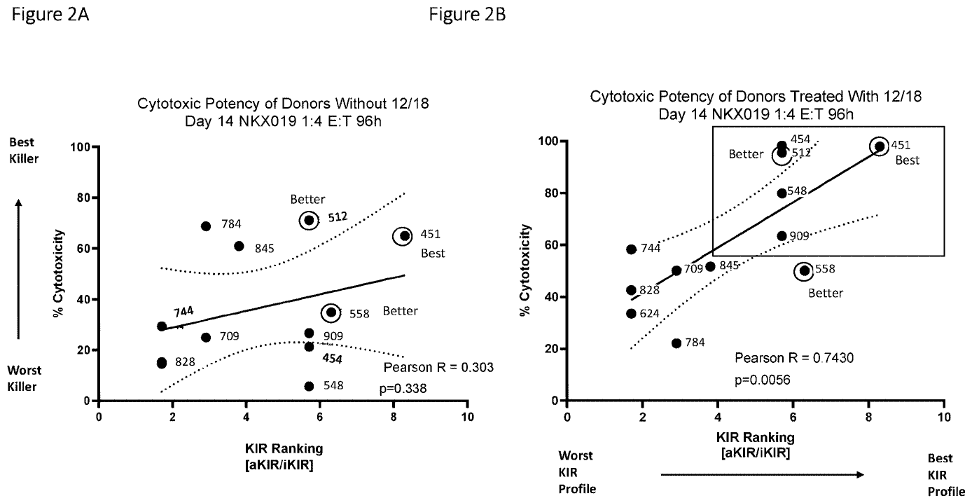

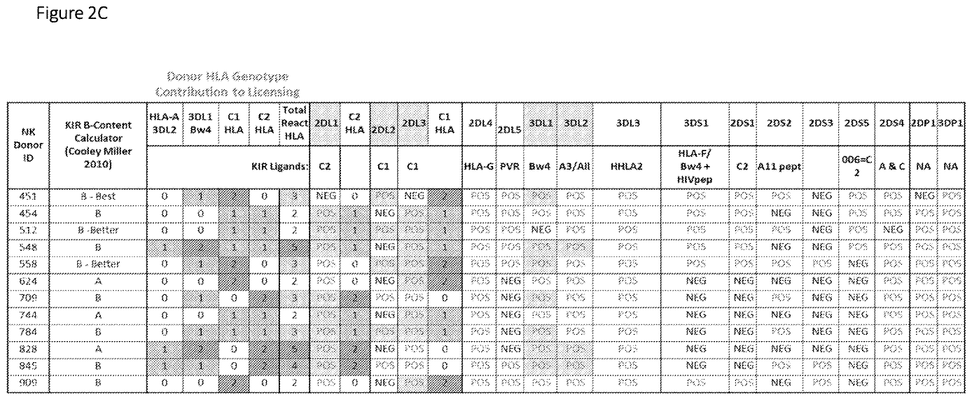

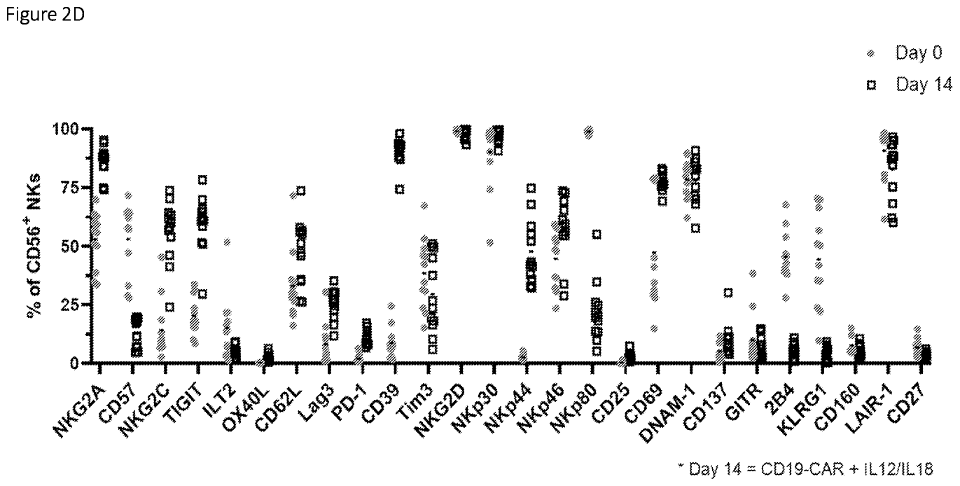

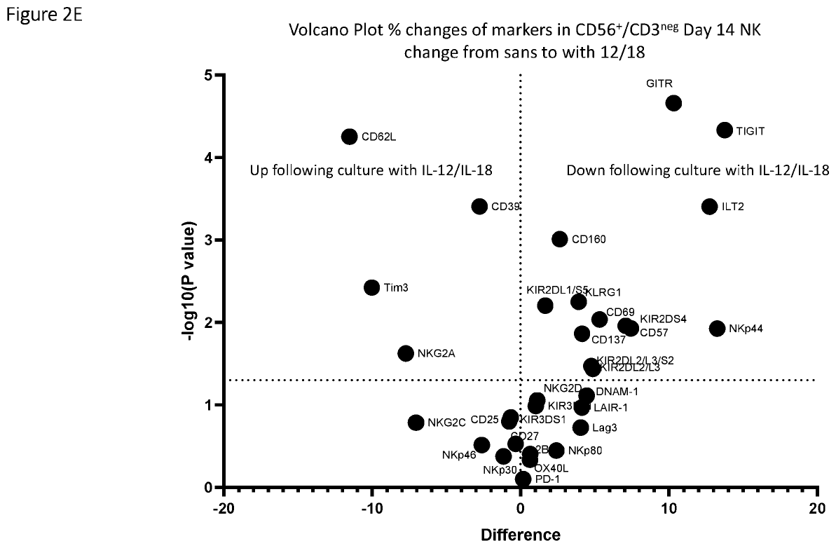

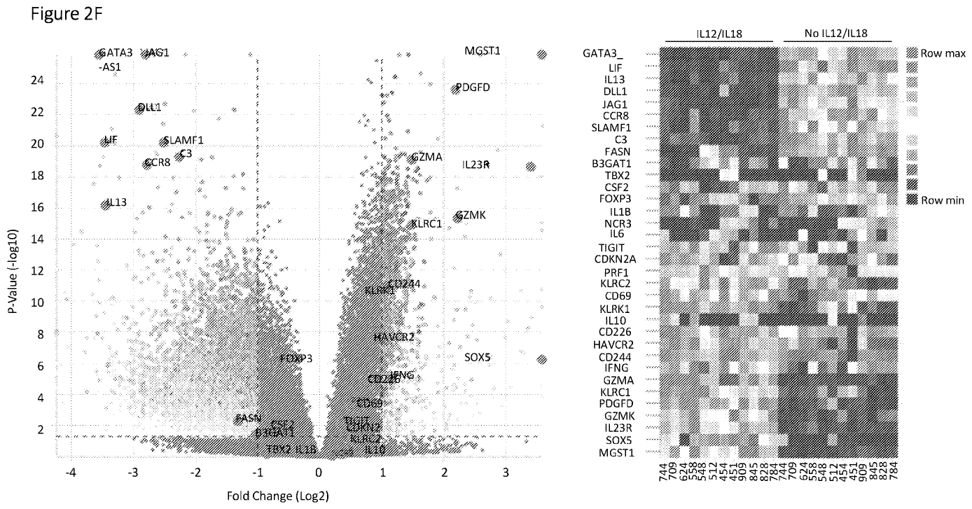

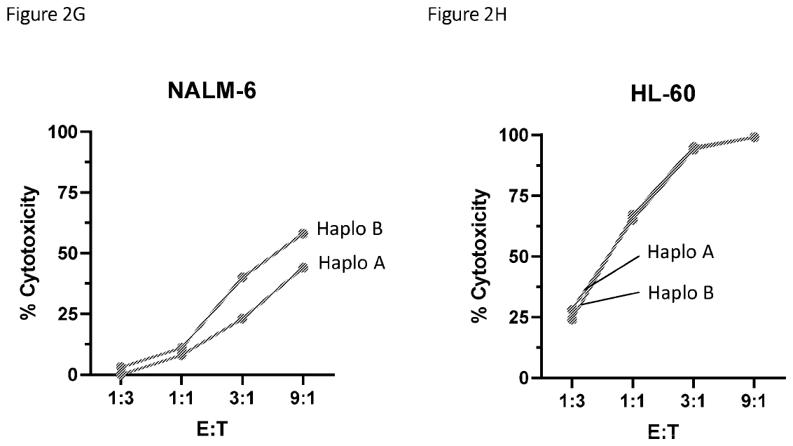

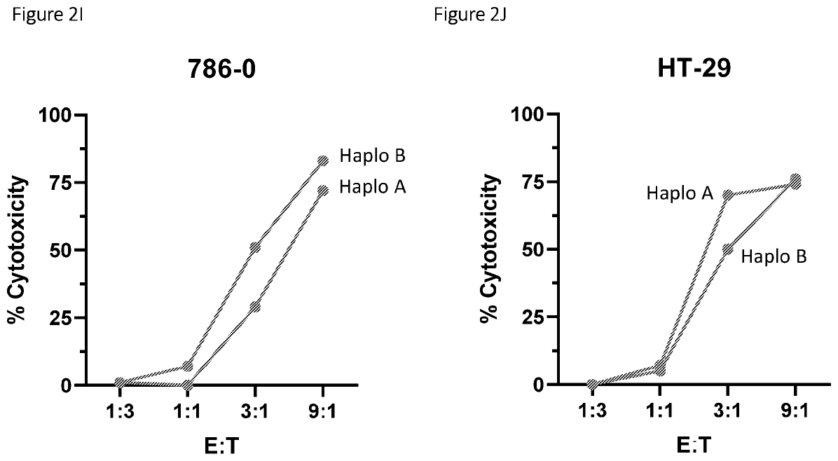

In view of the desire to screen candidate donors to identify preferred donors, the KIR profile of the donor cells was evaluated, according to methods disclosed herein. The data in FIG. 2A shows a correlation between the percent cytotoxicity exhibited and the total KIR haplotype of each donor (e.g., aKIR:iKIR ratio) when expanded without the use of IL12/IL18. FIG. 2B shows corresponding data when cells were expanded with IL12/IL18. Certain donors were identified in the data in FIG. 2A based on their performance in terms of cytotoxicity and “best” KIR profile. It is notable that these three donors maintained these categorizations when cultured with IL12/IL18, while also showing greatly increased cytotoxicity. The data in FIG. 2B demonstrate a highly significant correlation between the KIR profile and the cytotoxicity exhibited, which in several embodiments, allows for a candidate donor to be classified as a preferred donor based on their total KIR profile. To further characterize donor cells, a DNA-based high-resolution genotypic analysis of 12 donor cells (those from 2A and 2B) was undertaken. The analysis focused on the assessment of HLA & KIR genotype and also the KIR B content group was determined using an existing KIR Immuno-Polymorphism database (IPD-KIR). The analysis is summarized in FIG. 2C.

Further investigating the impact of expansion in the presence or absence of IL12 and IL18, NK cells from the twelve donors were genetically modified to express an anti-CD19-CAR-mbIL15 construct by retroviral transduction and expanded on K562 cells modified to express mbIL15 and 4-1BBL with or without soluble IL12/IL18 cytokines. Cells were characterized by flow cytometry on Days 0 & 14. These data show that the genetically modified NKs exhibit increased expression levels of activation markers, including activating NK receptors, for example TIGIT, Lag3, CD69, NKp30, NKp44, NKp46. Moreover, this assessment indicated a notable increase in NK memory associated markers, such as NKG2C, NKG2A, CD69. Additionally, there was a decrease in expression of the terminal differentiation marker CD57 by these expanded NKs. FIG. 2E shows a volcano plot of the changes detected in various NK cell markers at 14 days of expansion with, or without, IL12 and IL18. The upper left quadrant shows those markers that were increased after culture with IL12 and IL18, while the upper right quadrant shows those markers that were downregulated after culture with IL12 and IL18. FIG. 2F furthers this investigation into the impact of IL12 and IL18 on NK cells by evaluating expression of the genes encoding various markers of NK cell function by RNA sequencing (RNAseq). The addition of IL12 and IL18 to NK expansion cultures drives upregulation of genes associated with activation of NK cells. These data provide further evidence supporting the ability of culture conditions, including various cytokines, timings, feeder cells, and other variables disclosed herein, to impact the resultant expanded cells, not only in terms of raw cell numbers, post-expansion, but activation of the cells, and, as discussed herein, cytotoxic function and/or persistence of the expanded cells.

FIGS. 2G-2J continue the analysis of the interplay between donor identification leading to preferred, or even ideal, donor cells and the further enhancements that expansion conditions as provided for herein, which lead to further enhancements to NK cell cytotoxicity and persistence. There are two KIR primary haplotypes that can be found in humans: the group A haplotype and the group B haplotype. The group A haplotype has a fixed number of genes encoding inhibitory KIRs (with the exception of the activating receptor KIR2DS4). The group B haplotype has variable gene content, is generally more enriched in genes encoding activating receptors, and contains 1 or more of the following B-specific genes: KIR2DS1, KIR2DS2, KIR2DS3, KIR2DS5, KIR2DL2, and KIR2DL5. Certain studies in which pediatric acute lymphoblastic leukemia patients received human leukocyte antigen-haploidentical transplantation of ex vivo T-cell-depleted peripheral blood stem cells initially concluded that patients receiving transplanted cells from KIR haplotype B donors had an increased event-free survival. Unmodified KIR haplotype B donor NKs were treated with IL12/IL18 during expansion. These cells, even without genetic engineering to express a CAR, were able to effectively kill tumor cells across different tumor types, including NALM-6—B cell leukemia (2G), HL-60—myeloid cell leukemia (2H), 786-0—renal cancer (2I), and HT-29—colorectal adenocarcinoma (2J). 6 donors were tested: 2 haplotype A and 4 haplotype B, group averages are shown. These data show that, despite the possibility that a starting KIR B haplotype is preferred in some instances, whether the donor cells are KIR haplotype A or B, the use of IL12 and IL18 during expansion imparts to both KIR haplotypes an ability to exhibit cytotoxic effects against a variety of target cells. In some instances where the exhibited cytotoxicity is lower than desired, cells can be further manipulated (e.g., engineered to express a CAR or other modification to synergistically increase the cytotoxicity and/or persistence of the resultant NK cells.

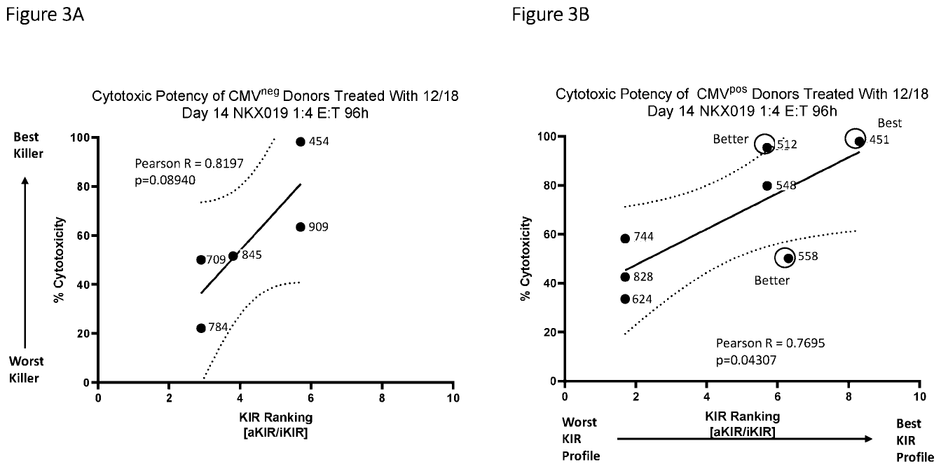

Despite the potential impact of a prior cytomegalovirus infection to impart to immune cells, such as NK cells, an enhanced cytotoxicity due to induction of a memory or memory-like phenotype, FIGS. 3A and 3B demonstrate that the correlation of donor potency and KIR profile is not impacted by CMV status. FIG. 3A shows the cytotoxicity: KIR profile correlation for CMV-negative donors and FIG. 3B shows the same data for CMV-positive donors. While the indicated donor cells who were, at least in this non-limiting example experiment, the highest performers and were CMV-positive, FIG. 3B shows lower performers in the CMV-positive group, while FIG. 3A shows high cytotoxicity-exhibiting cells in the CMV-negative group.

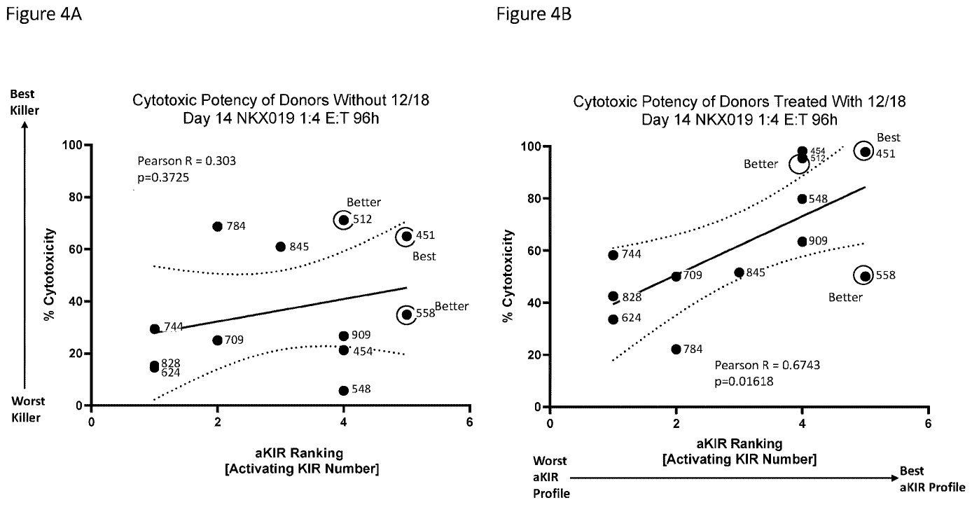

In order to more fully elucidate the assessment of the KIR profile of a donor on the predicted cytotoxicity of that donor's cells, correlations were performed that involve the cytotoxicity versus the activating KIR profile (as opposed to the total profile). These data are shown in FIGS. 4A-4B. FIG. 4A shows the cytotoxicity-activating KIR correlation for donors whose cells were expanded without IL12/IL18, while FIG. 4B shows the data related to expansion with IL12/IL18. As can be seen, there is still a significant correlation between the cytotoxicity of the cells expanded with IL12/IL18 and the activating KIR profile, but it is not as significant as when the total KIR profile is evaluated. However, according to some embodiments, assessment of the activating KIR profile is sufficient to categorize a donor as a preferred donor and move their cells into the expansion processes disclosed herein employing IL12/IL18. In contrast, FIGS. 5A-5B show cytotoxicity data as a function of iKIR ranking without (5A) or with (5B) IL12/IL18 used in the expansion process. As can be seen from the data, whether or not IL12/IL18 were used in the expansion process, the assessment of only the iKIRs present on the NK cells does not correlate with the ultimate cytotoxicity (despite the pro-cytotoxicity impact IL12/IL18 provide). Thus, in several embodiments, assessment of candidate donors relies on at least the evaluation of the aKIR profile, and in some embodiments, both the aKIR and iKIR profiles are determined.

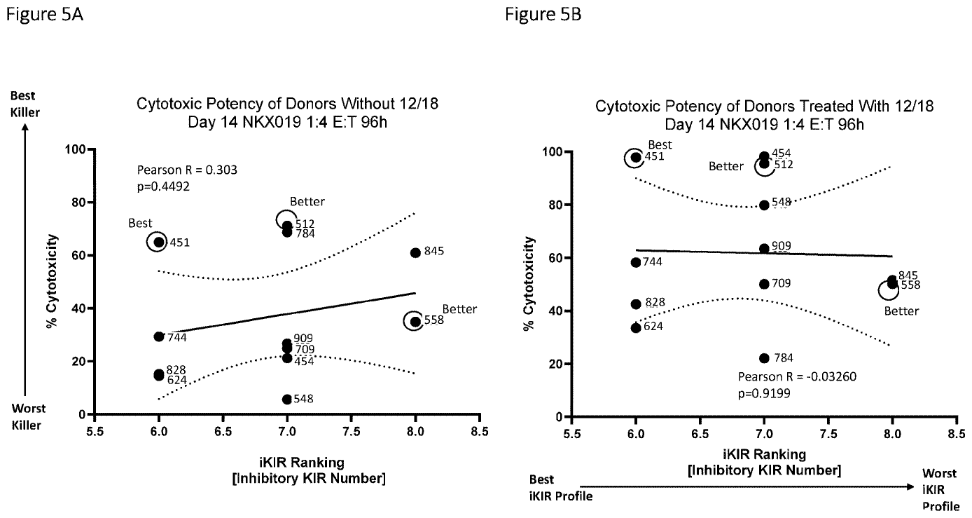

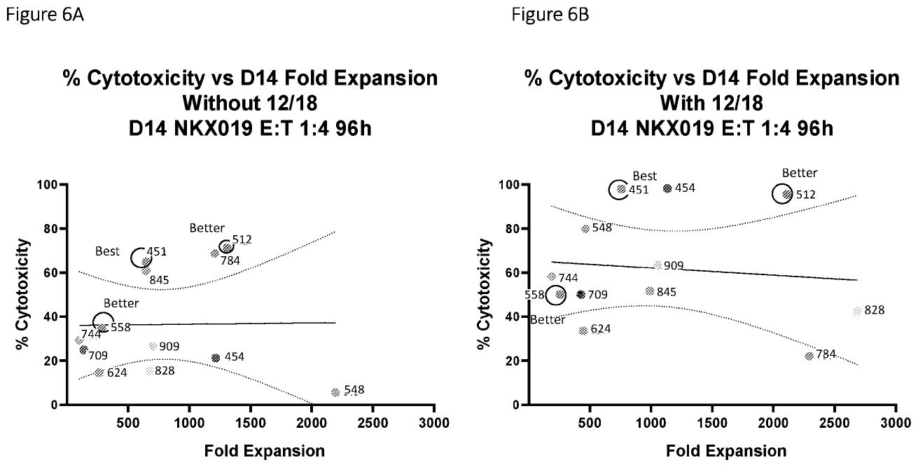

Further evaluation into the relationship between potential for expansion and cytotoxicity was undertaken. FIGS. 6A-6B show the relationship between cytotoxicity and fold expansion of cells without (6A) and with (6B) IL12/IL18 used in the expansion process. These data indicate that, for a given donor, whether IL12/IL18 are used in the expansion process or not, the ability of a given donor's cells to expand in culture is independent of the cytotoxicity towards tumor cells of those expanded donor cells. For example, cells from donor 828 exhibited about 600-700 fold expansion in the absence of IL12/IL18 and less than 20% cytotoxicity. When cultured with IL12/IL18, cells from donor 828 were expanded over 2500 fold, but increased in cytotoxicity to about only 40%. Cells from donor 451 exhibited about 600-700 fold expansion under either condition, but with the use of IL12/IL18 in the expansion, increased from just over 60% cytotoxicity to nearly 100% cytotoxicity. As another example, cell from donor 512 exhibited enhanced expansion and cytotoxicity in the presence of IL12/IL18. Thus, according to some embodiments, the ability of a donor cell to be expanded robustly in culture does not necessarily mean that those cells will be effective at eliminating tumor cells. However, as discussed above, evaluation of aKIR/iKIR does, in several embodiments, allow a prediction of future cytotoxicity. In several embodiments, the expansion process allows several hundred-fold expansion (e.g., at least about 100-fold, about 200-fold, about 300-fold, about-400 fold, about 500-fold, about 600-fold, about 700-fold, about 800-fold, about 900-fold, about 1000-fold, about 1500-fold, about 200-fold, or more (including amounts between those listed).

### Example 2—Assessments of Donor Cell Cytotoxicity Performance Separation

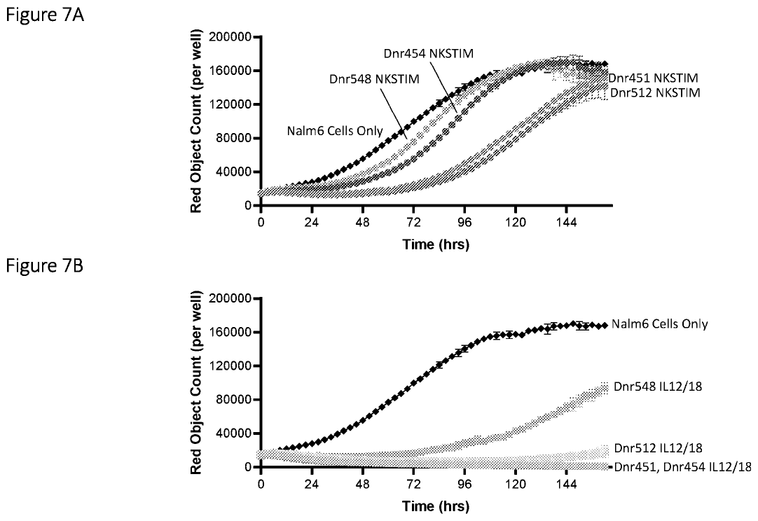

To assist in identification of donor cells exhibiting particularly desired levels of cytotoxicity studies were undertaken to determine how to separate the donors based on performance (e.g., cytotoxicity and/or expansion). These studies were also intended to help elucidate the effects of use of IL12/IL18 in the expansion process on the expanded cells. Cells were also cultured under conditions employing stimulatory molecules in the media, but utilizing a different overall expansion process (referred to in the Figures as NKSTIM, whereas the methods as provided for herein are labeled in the Figures as IL12/18). Cells were from selected donors were transduced with a non-limiting example of an anti-CD19 CAR and tested for their cytotoxicity against Nalm6 tumor cells at 14 days after completion of expansion of the cells. In FIGS. 7A-7B a 1:4 effector:target ratio was used. FIG. 7A shows the cytotoxicity profile of the cells when expanded using an alternative expansion approach and FIG. 7B shows the cytotoxicity profile of the cells when expanded using the methods disclosed herein. Overall, it is notable that the growth curves in FIG. 7B (representing tumor cell growth) are substantially muted as compared to those in 7A, indicating that the IL12/18 expansion conditions have resulted in cells that exhibit greater cytotoxicity against target cells. Nearly all donor cells using the IL12/18 expansion conditions disclosed herein nearly completely controlled tumor growth throughout the experiment and beyond the Nalm6 point of plateau. In particular, the cells from donor 454 (shown labeled with a light arrow in 7A and 7B) moved from completely controlling tumor growth in 7B to allowing almost as much tumor growth as control when NKSTIM expansion was used (7A). To further elucidate the most effective performing donor cells (in terms of cytotoxicity) another experiment was performed, but using an effector:target ratio of 1:8 (so twice as many tumor cells per engineered cell). These data are presented in FIGS. 8A (NKSTIM) and 8B (IL12/18 per embodiments disclosed herein). While the greater number of tumor cells caused there to be more tumor growth in the IL12/18 expanded group, those donors still outperformed those expanded using NKSTIM. Moreover, this lower E:T ratio allowed for separation of the most effective donor cells (donor 454 and 451; see double headed arrow).

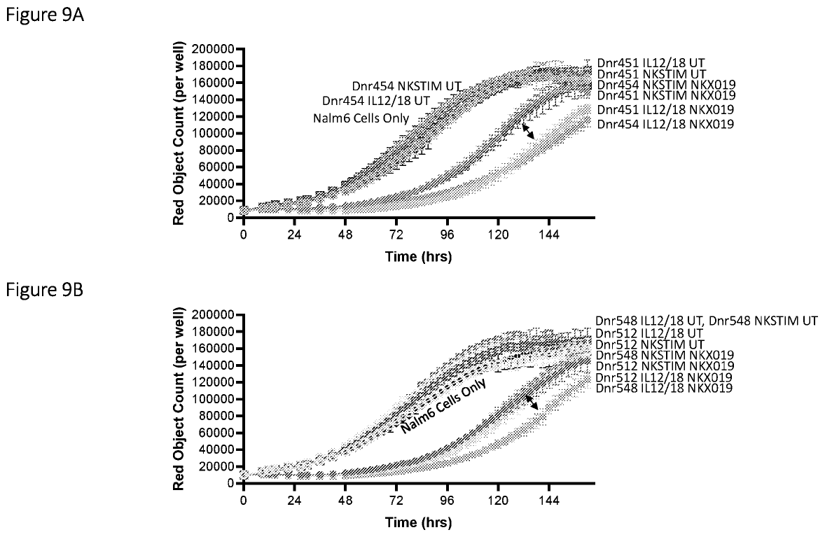

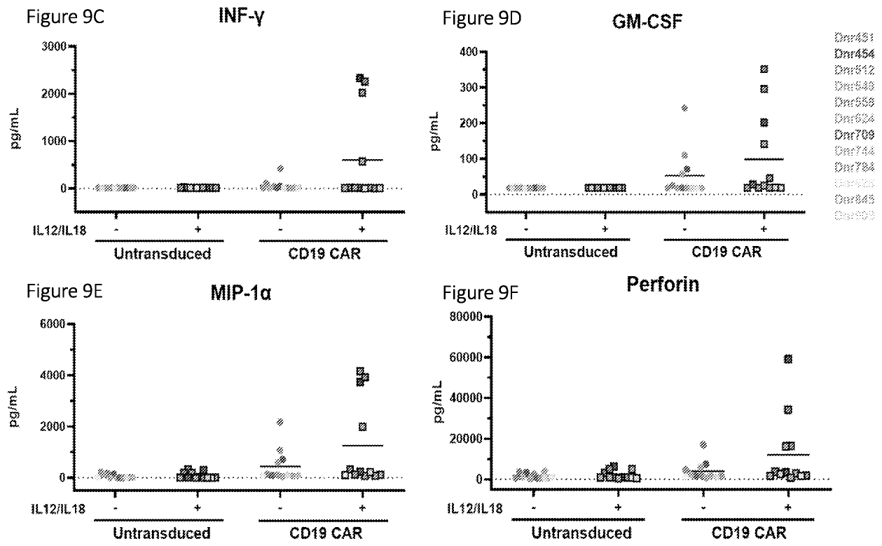

Expanded cells were evaluated for their cytotoxic persistence at longer time frames as well. FIGS. 9A and 9B show data for Nalm6 tumor challenge 21 days after completion of expansion. FIG. 9A shows data for donor 451 and 454 using either NKSTIM or IL12/18 expansion and with (NKX019) or without (“UT”-untransduced) CAR expression. Here the E:T ratio was again 1:4. While anti-tumor activity is reduced across all donors, even at 21 days post-expansion, the use of the IL12/18 expansion conditions still drove enhanced cytotoxicity in three of the four donors tested (see boxed legend and arrows).

To further evaluate the cytotoxic effects of the engineered and expanded NK cells, supernatants were collected from a prior experiment in which engineered NK cell were tested for their cytotoxicity against NALM-6 cells, as a non-limiting embodiment of a target cell (i.e., cells as tested in NALM-6 tumor cytotoxicity assays (FIGS. 1B-1C). The supernatants were evaluated on a multi-plex panel consisting of 11 analytes. As shown in FIGS. 9C-9F, increased levels of IFN-g (9C), GM-CSF (9D), MIP-1a (9E), and Perforin (9F) were detected in IL12/IL18 expanded CD19-CAR transduced NKs. These data appeared to be consistent with the cytotoxic potency ranking of individual NK donor, see, e.g., FIGS. 2A-2C. Taken together, these data support the process of identifying a candidate donor having a desired KIR expression profile as a starting point for collecting donor cells, to be modified/expanded according to embodiments disclosed herein, in order to achieve enhanced anti-tumor effects.

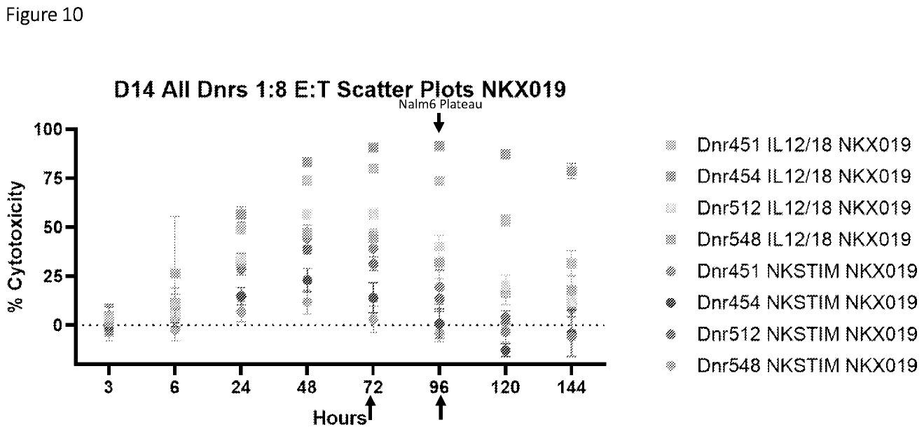

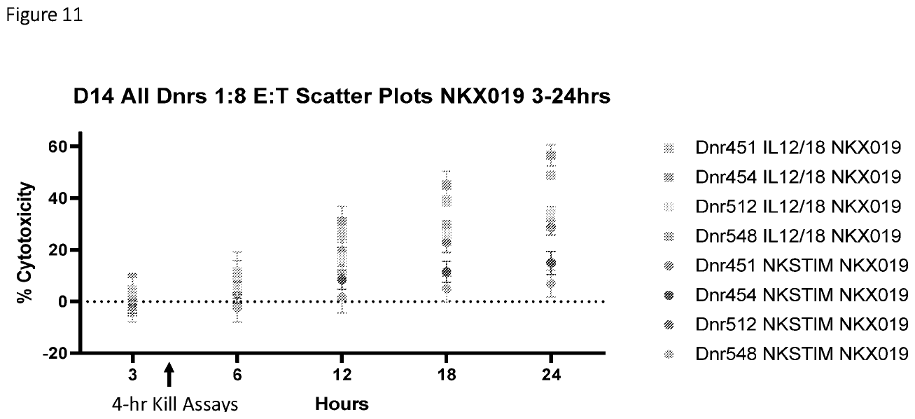

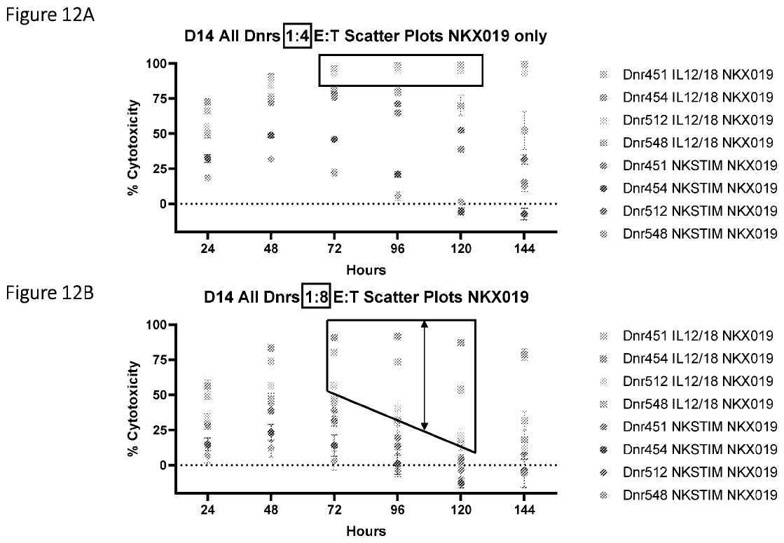

FIG. 10 shows scatter plot data of all donors tested after expansion using the IL12/18 or NKSTIM conditions and the 1:8 E:T ratio in order to help elucidate the more potent cells. FIG. 11 shows these data re-binned on earlier time points, which can help identify the more potent cells at earlier times. In several embodiments, the donor biology (e.g., KIR profile) correlates, as discussed above, with the potency as later assayed, reflecting the consideration of both donor profile and ability for expansion and responsiveness to stimulating molecules, such as IL12 and IL18, as a driver of obtaining unexpectedly effective and persistent cells for therapies. FIGS. 12A-12B show how various experimental conditions can facilitate separation of donors based on measured cytotoxicity. FIG. 12A shows cytotoxicity assessment when a 1:4 E:T ratio is used. The data curves, even when shown as scatterplots, make it challenging to separate the donors with the highest degree of cytotoxicity, due to data compression. Thus, FIG. 12B shows the same experimental setup with the exception of a 1:8 E:T ratio. As can be seen in the scatterplot, the lower E:T ratio allows for the top performing donor cells to be separated along the Y axis (denoting % cytotoxicity), while compressing the lower performing donor cells. These data indicate that, according to embodiments disclosed herein, expansion of donor cells using multiple pulses of feeder cells and supplementation with IL12/IL18 results in enhanced cytotoxicity of the resultant expanded cells.

### Example 3—Evaluation of Expansion, Cryopreservation and Cytotoxicity

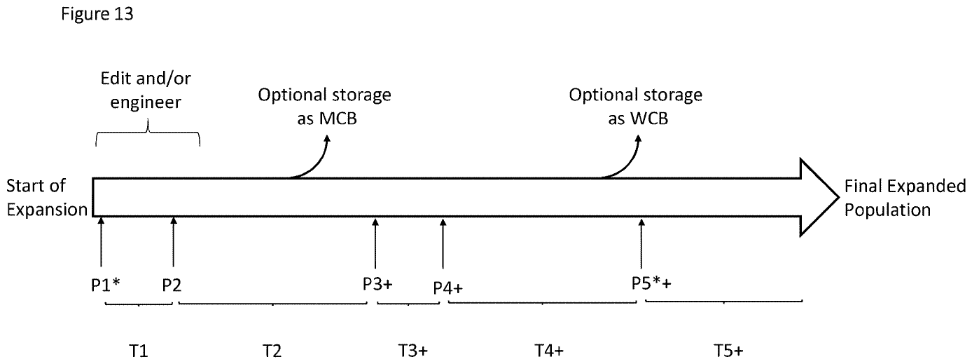

As disclosed herein, various methods for substantially expanding immune cells, such as NK cells, are provided. As discussed above, in several embodiments, these methods impart particularly effective characteristics to the resulting cells, such as cytotoxicity. Experiments were performed to examine embodiments of the expansion process and other impacts on the resulting cells. FIG. 13 shows a schematic depiction of a non-limiting embodiment of an expansion process provided for herein. As shown, the process moves from the start of expansion (using either freshly donated cells, or cells that were previously cryopreserved) to generation of a final expanded product (e.g., cells ready to be stored or administered to patients). As shown, aliquots of cells can be removed and stored as either a master cell bank (MCB) or working cell bank (WCB) for future use, for example after cryopreservation. Alternatively, the cells can be run through the process without generation of cell banks. As discussed above the cells to be expanded are co-cultured with feeder cells, as disclosed herein, with each fresh batch of feeder cells being a “pulse” or “P”. As shown in the non-limiting schematic of FIG. 13, five pulses are used in this experiment, though additional pulses could be used (as indicated by the “+” on each of the P3-P5). The time (“T”) is also indicated and can vary between the pulses, or can be consistent between one or more pulses (e.g., T1 and T3+ may optionally be the same duration). As indicated by the “*” in FIG. 13, one or more of the pulses include supplementation of the media with at least IL12 and IL18, as disclosed herein. In several embodiments, IL2 is also included. Media changes (using either IL2-free or IL2-supplemented media) are not shown and can be performed based on the visual health of the cells being expanded, the relative cell density, or other measures within ordinary skill. As shown in this non-limiting embodiment, the cells to be expanded are gene edited and/or genetically engineered early in the expansion process. In several embodiments, the gene edit occurs prior to the expansion process beginning (e.g., day-1 in the process). In several embodiments, as discussed above, donors are selected based on assessment of at least the aKIR profile of their cells. The donors used in this set of experiments were selected based on two of them (451 and 454) having aKIR/iKIR ratios that exceeded the threshold of 3. Donor 744 did not exceed that threshold.

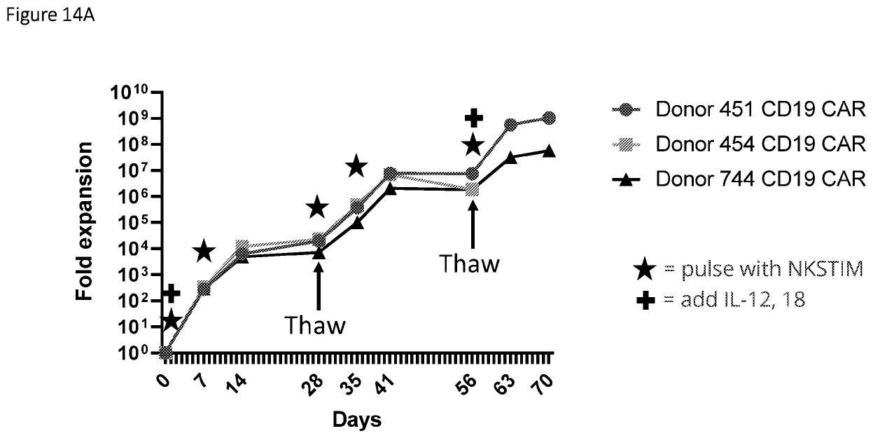

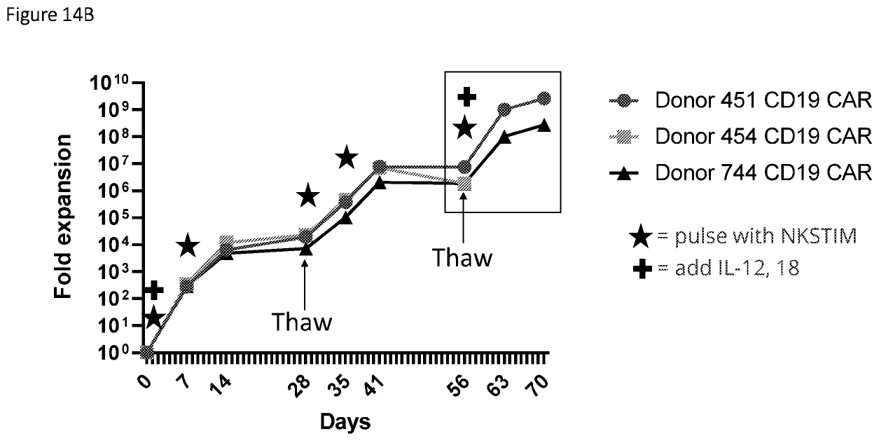

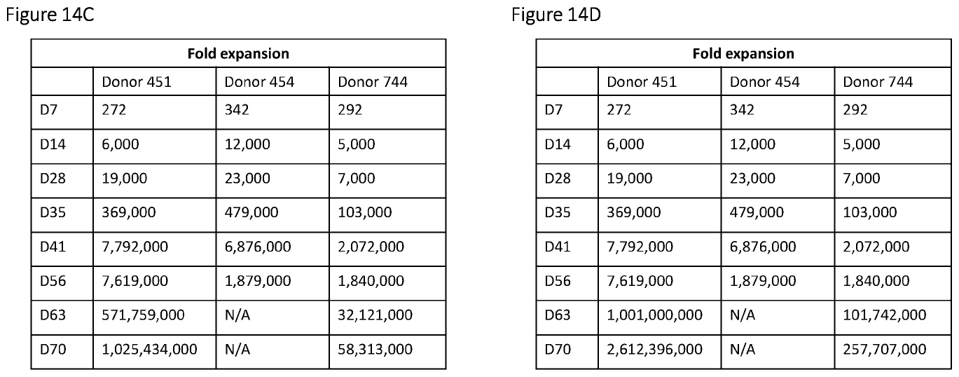

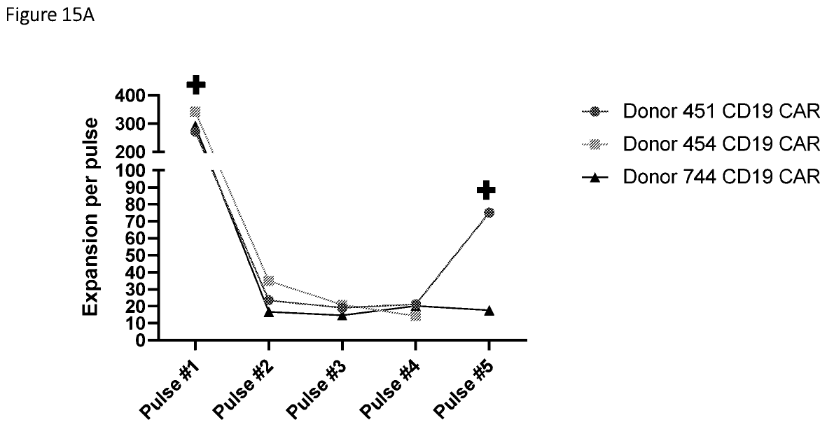

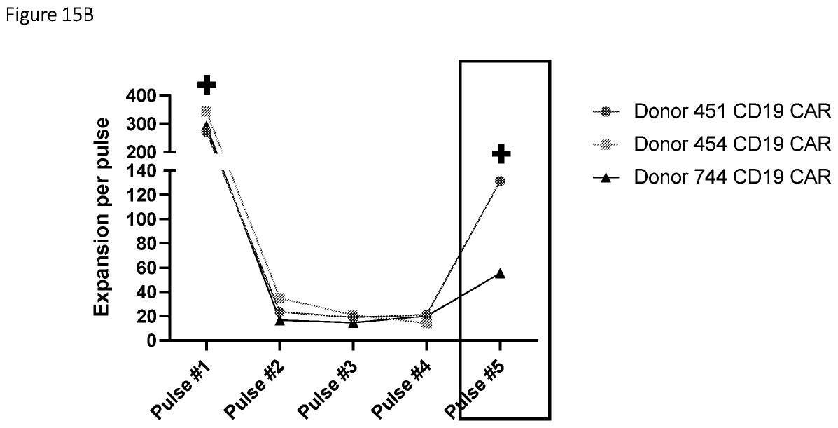

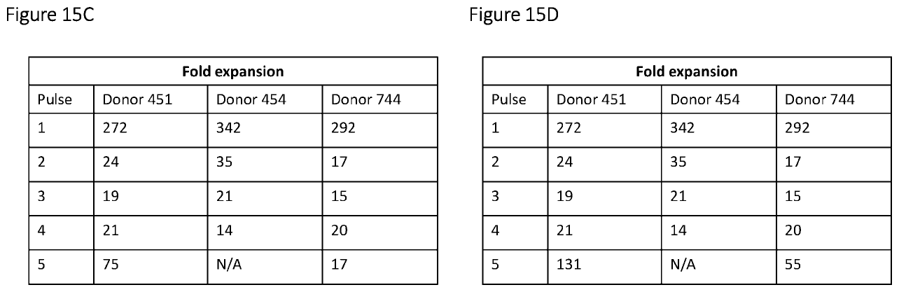

FIG. 14A shows a line graph depicting the fold expansion of cells from the three donors using a five-pulse process, as indicated. Cells were cryopreserved after pulse 2 and 4 and then thawed prior to pulse 3 and 5, respectively. IL12 and IL18 were used at pulse 1 and 5. FIG. 14B shows data from an additional experiment, where the first 4 pulses are the same data as FIG. 14A, but a new batch of cells was thawed and subjected to pulse 5 (new data is in the box). As shown, this pulsing or “multistim” process yielded unexpectedly robust expansion, with the data from FIGS. 14A and 14B being tabulated in FIGS. 14C and 14D, respectively. As shown in those tables, the least expanded cells at day 56 of the process were those from donor 454, yet even those cells had achieved nearly 2 million fold expansion (see 14C). The cells from the other two donors achieved over 1 billion-fold expansion (451) and nearly 60 million-fold expansion (744). With a repeat of the final pulse (new data is below the arrow in 14D) expansion was achieved at over 2.5 billion-fold expansion (451) and over 275 million-fold expansion (744). FIGS. 15A-15D show data as to the degree of expansion for each pulse of the process. As with FIG. 14, FIG. 15B shows the same data as for 15A for the first four pulses, with a new replicate of the experiment performed at pulse 5 (likewise for 15C and D). These data demonstrate that the process results in robust early expansion and the consistent expansion through pulses 2-4, with a possible secondary period of elevated expansion after pulse 5. These data are tabulated in FIGS. 15C and 15D, which show expansion exceeding 270-fold at pulse 1, ranging from about 15 to 30 fold at pulses 2-4, and returning to elevated levels at pulse 5. These data show that, as according to several embodiments, the use of IL12 and IL18 at pulse 5 causes the cells to enter an additional period of expansion, which results in a substantial number of cells that are available for clinical use (optionally preceded by cryopreservation).

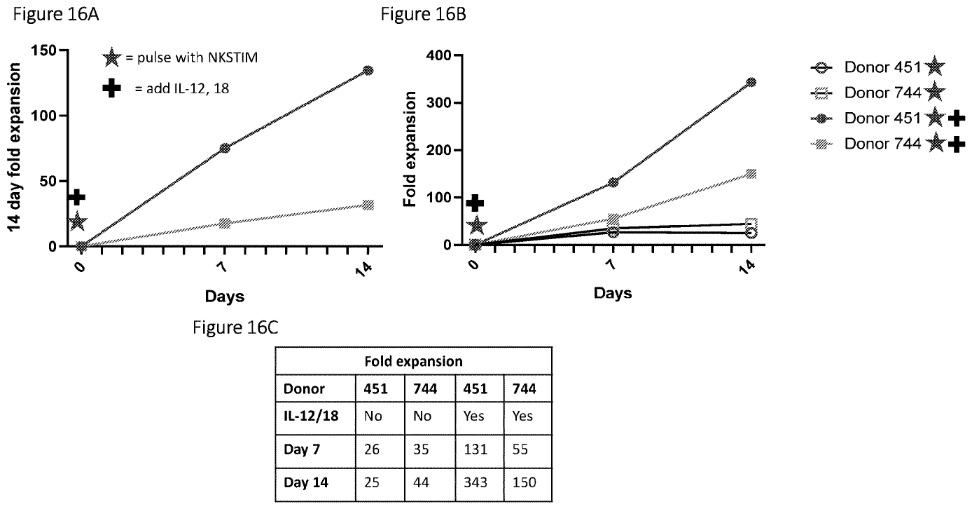

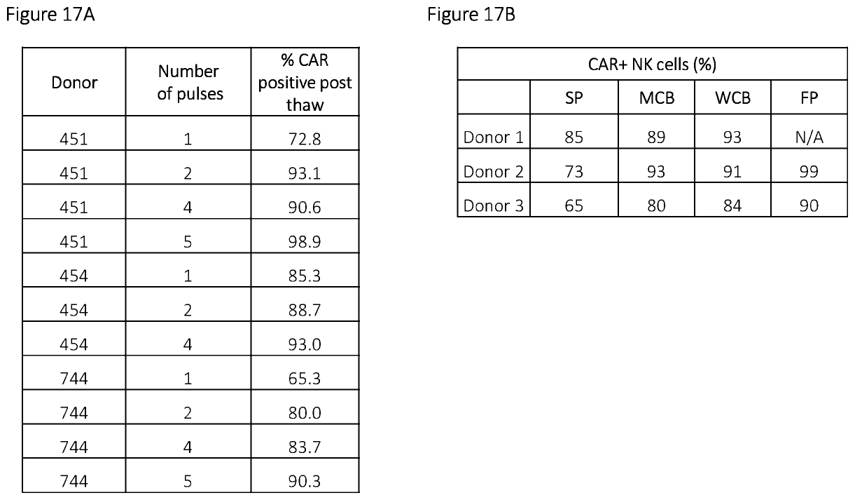

Further experiments were conducted to elucidate the impact of the IL12 and IL18 added at pulse 5. FIG. 16A shows expansion data for the final 14 days of culture (from pulse 5 to final product). FIG. 16B shows similar data from another replicate in which IL12 and IL18 were either present (solid) or absent (open). FIG. 16C tabulates this data which shows that the presence of IL12 and IL18 marked enhances the expansion of the cells at the final pulse. As the expanded cells are transduced to express a CAR early in the expansion process, FIG. 17A shows that, importantly, over 90% of the cells still express the CAR at the close of expansion. It should be noted that these cells were also genetically edited to reduce expression of CISH, according to embodiments disclosed herein. This advantageously results in a highly expanded cell population that is enriched with respect to CAR positivity. FIG. 17B breaks down similar expansion data based on the stage of production according to methods disclosed herein. SP refers to expansion of NK cells using K562 cells modified to express mbIL15 and 4-1BBL as feeder cells and including soluble IL12 and IL18 in the culture media (termed “NKSTIM”; see, for example International Patent Application No. PCT/US2020/044033, filed Jul. 29, 2020, the entire contents of which is incorporated by reference herein). MCB refers to Master Cell Bank, WCB refers to Working Cell Bank, and FP refers to Final Product (see e.g., FIG. 13). These data show that at each stage of the expansion process (other than Donor 1 at FP), significant percentages of the cells retain expression of the transduced CAR, meaning that even after significant expansion, the expression of the CAR is persistent, and a substantial portion of the post-expansion cells remain therapeutically relevant.

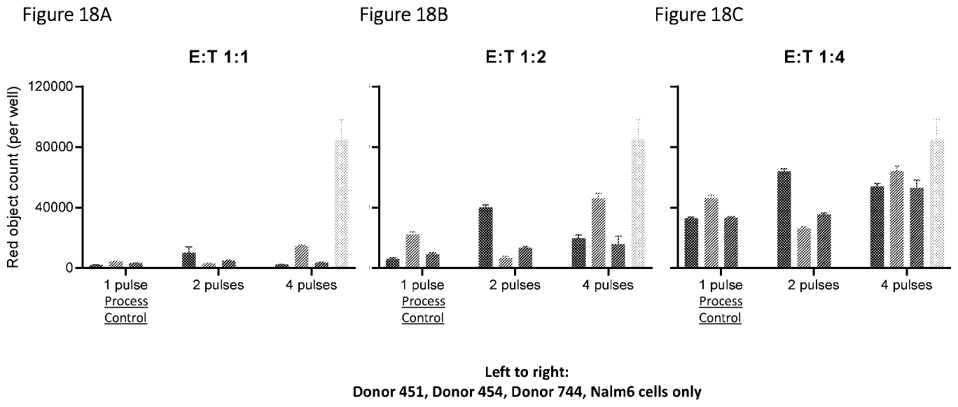

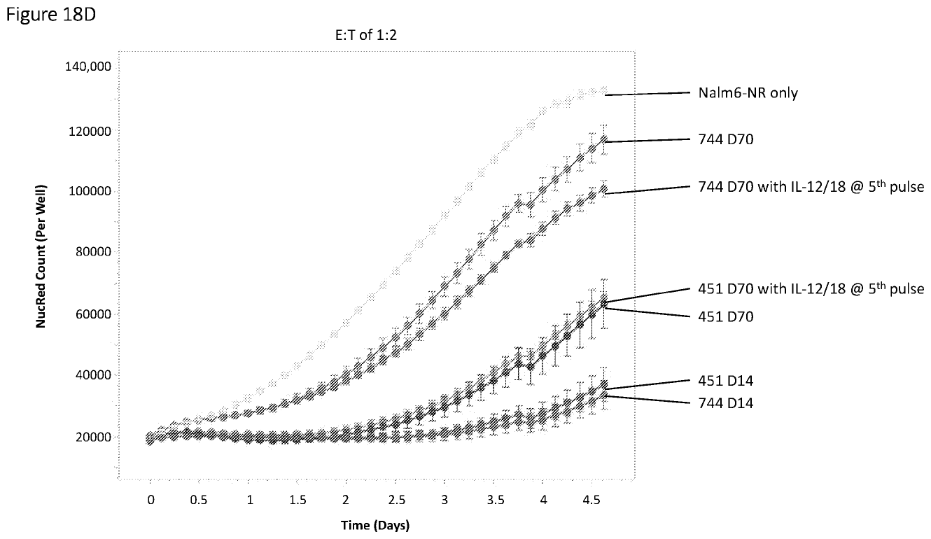

Moreover, the cell population post-expansion has been demonstrated to be functional (e.g., cytotoxic) during, and after, expansion. FIGS. 18A-18C show summary data related to the cytotoxicity of the cells at either 1:1 (18A), 1:2 (18B) or 1:4 (18C) E:T ratios with assays being performed at 1, 2, and 4 pulses (pulse 1 being a control expansion process using feeder cells and IL12/IL18. Red object count (tumor cell) is substantially lower than control (Nalm6 alone) at all pulses across all E:T, indicative of potent cells. FIG. 18D shows tumor growth curves when cells at the completion of the expansion process were co-cultured with the Nalm6 tumor cells when IL12/18 were included, or not, at the final expansion pulse. These data show that, while Day 14 process control cells effectively control tumor growth, the cells expanded for the full process also demonstrate a favorable profile, particularly for donor 451 (aKIR/iKIR ratio above threshold). Donor 744 (aKIR/iKIR below threshold) allowed Nalm6 growth towards the end of the experiment, but also shows the positive impact of IL12/IL18 on cytotoxicity (the lower curve for donor 744). Thus, according to embodiments disclosed herein, immune cells, such as NK cells, that are expanded according to embodiment disclosed herein retain a high degree of cytotoxicity. Coupled with the much larger number of cells, this allows for multiple patient doses to be generated and stored, thereby facilitating effective allogeneic cell therapy.

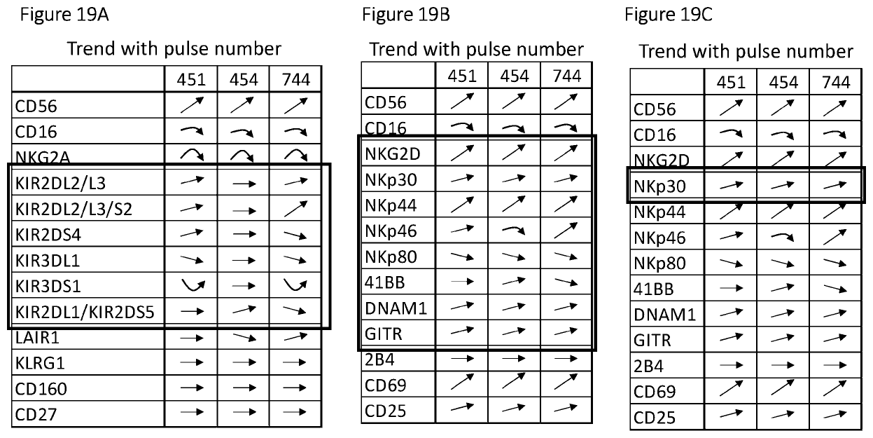

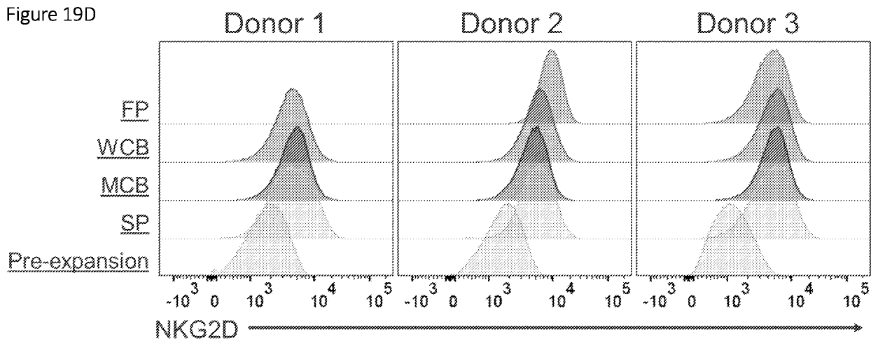

Looking further into the potential mechanism of action underlying the cytotoxicity imparted to the expanded cells, various phenotypic assays were performed on the cells during the expansion process. FIG. 19A shows the general trend of increased KIR expression with increasing pulse number. Both aKIR and iKIR expression seemed to trend upwards. FIG. 19B shows data that indicates that several activating receptors increase in expression with pulsing during expansion. Notable among these is the expression of NKp30 (FIG. 19C) which shows the trend for increased expression over the initial four pulses of the expansion. NKp30 is one of the natural cytotoxicity receptors, a family of immunoglobulin (Ig)-like NK cell activation receptors, that has been shown on human NK cells to be key receptors in tumor immunity. These data indicate that many activating receptors undergo upregulated expression during expansion. FIG. 19D summarizes, for three donors, the expression of the NKG2D surface expression on NK cells. As can be seen for each donor, as compared to a pre-expansion control (bottom row), expansion of NK cells with the Standard Procedure (“NKSTIM” as discussed above, see for example International Patent Application No. PCT/US2020/044033, filed Jul. 29, 2020, the entire contents of which is incorporated by reference herein) results in greater expression of NKG2D (right shift of the curve). Similar trends were observed for NKp30, NKp44, NKG2C, DNAM-1, KIR2DS4, and KIR3DS1 in most donor NK cells. During the progression through the multi-phase expansion processes embodied herein (Master Cell Bank to Working Cell Bank to Final product), the expression of NKG2D generally continues to increase (right shift of curve moving up from MCB to WCB to FP). As discussed in more detail below, the continued increase in activating receptors is believed to engender the cells with long lasting cytotoxic potency against target tumor cells, even after significant (and for some donors pre-terminal expansion limits) expansion. As a result, in several embodiments, the expansion methods disclosed herein result in a sizeable, cytotoxically potent, and persistence population of cells for cancer immunotherapy.

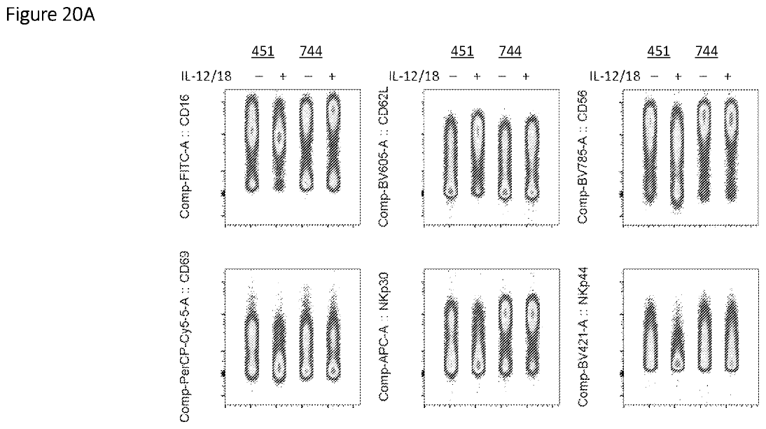

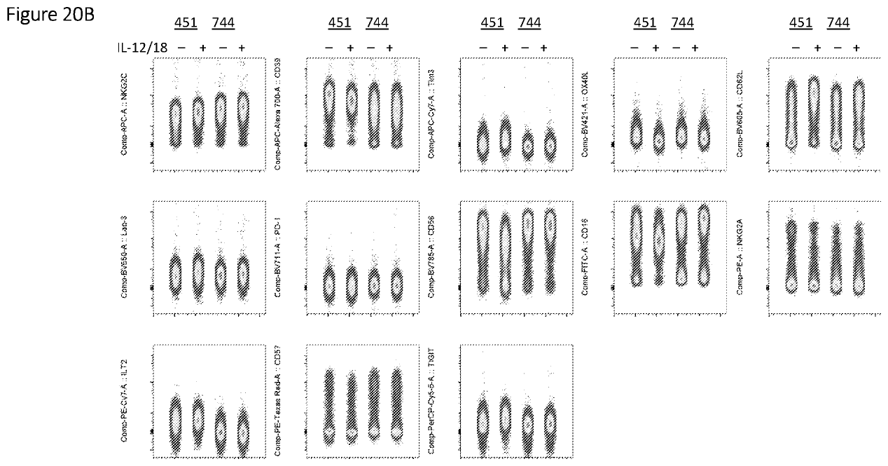

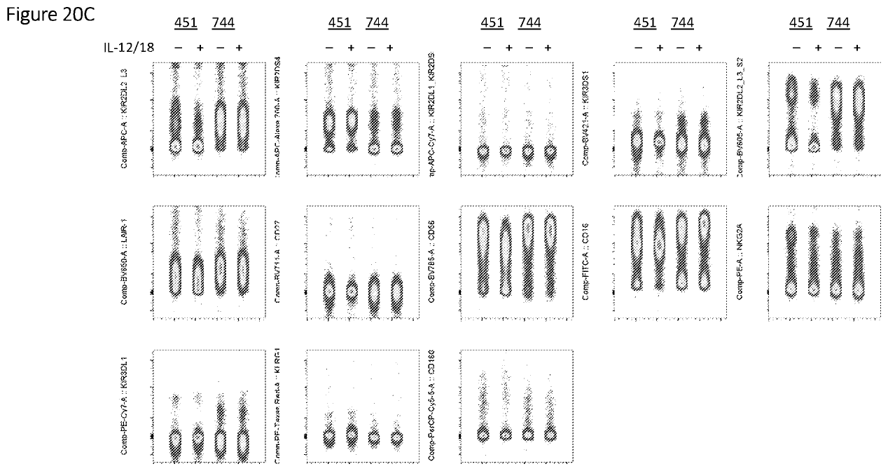

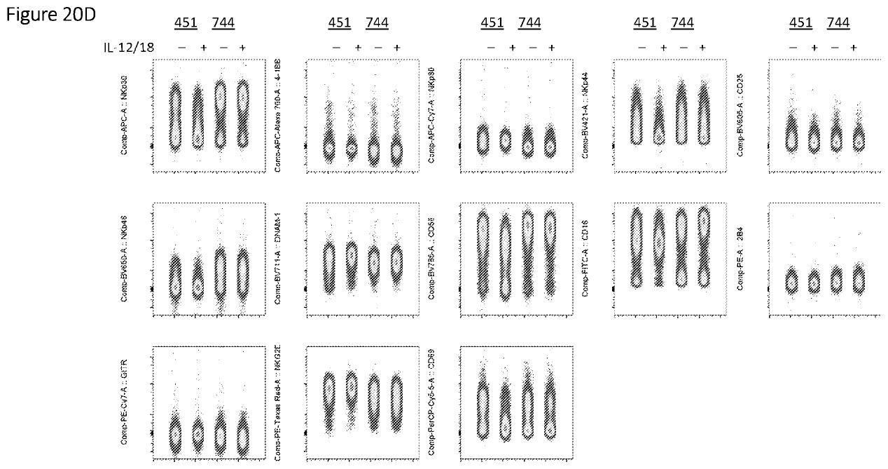

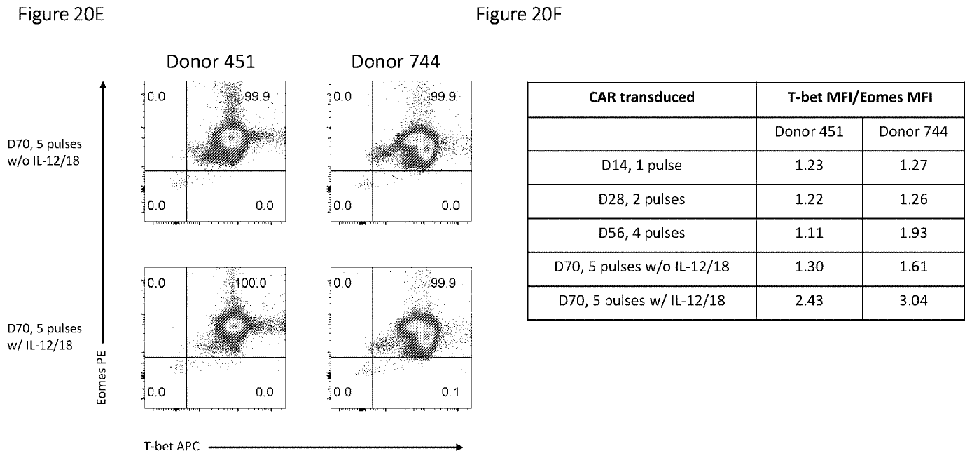

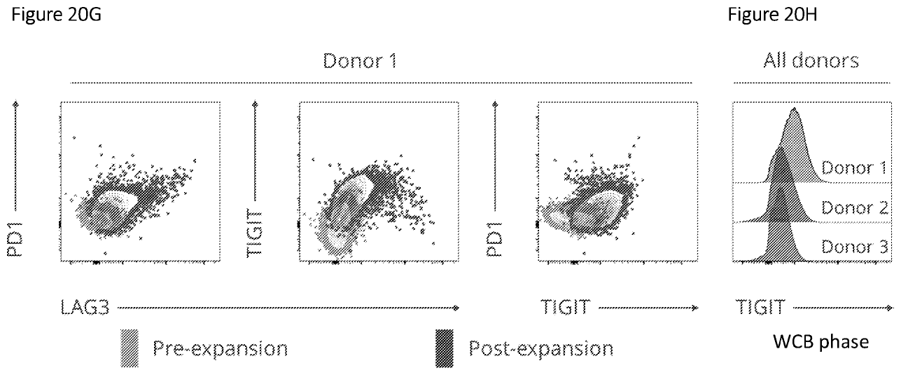

FIGS. 20A-20F show expression of various markers on the NK cells of two donors when expanded with or without IL12/IL18 at the final pulse. There are several notable differences (see FIG. 20A, which is a selection of data from FIGS. 20B-20C) in expression when IL12/IL18 is present, such as the increased expression of CD62 ligand (CD62L), which, at least on T cells, functions as an activation marker, as opposed to a memory marker. Memory T cells are known to be less responsive to tumor cells as compared to naive T cells, thus the IL12/IL18 induced increase in CD62L may be resulting in a population of more active NK cells. FIGS. 20E and 20F show the impact of Il12/IL18 on expression of T-bet and Eomes, two T-box transcription factors that regulate NK cell development and activity. The elevated expression of these two transcription factors may be involved in the enhanced cytotoxicity exhibited by cell expanded using Il12/IL18 at the final pulse. FIGS. 20G-20H show data related to the expression of markers of exhaustion on NK cells. FIG. 20G shows plots of various markers for Donor 1, who was the donor whose cells did not expand to the FP phase (as compared to Donors 2 and 3, which expanded over 2 billion and 200-million fold, respectively), though they did expand ~7 million-fold before undergoing contraction. PD-1, LAG3 and TIGIT are established markers of exhaustion in T cells, and the experiment discussed here was to determine how the expression of these markers changed on NK cells during expansion, in particular for Donor 1, whose expansion lagged behind that of Donor 2 and Donor 3. In the left panel of FIG. 20G, there is an increase in both PD-1 and LAG3 (comparing pre-expansion to post-expansion), with a slightly greater LAG3 increase (evidenced by the shift of the plot up but also slightly more to the right). The central panel of FIG. 20G shows a fairly dramatic increase in TIGIT expression as compared to LAG3 (vertical shift of plot) and likewise, the right panel of 20G show a significant right shift (TIGIT) as compared to vertical upshift of PD-1. These data suggest that TIGIT expression increasing based on NK cell expansion may drive an exhaustion-like phenotype that could be responsible for the limited expansion of Donor 1. FIG. 20G shows TIGIT expression at the WCB phase for all three donors, and Donor 1 notably exhibits a greater expression of TIGIT at that stage of expansion. Thus, in several embodiments, cells may be optionally evaluated during expansion (e.g., at the WCB phase, or some other time prior to FP generation) for TIGIT expression levels. In several embodiments, an elevated TIGIT expression level can result in termination of the expansion of those cells, on the premise that the overall expansion of those cells will not reach the full potential of the methods disclosed herein (e.g., for a donor expressing lower TIGIT levels).

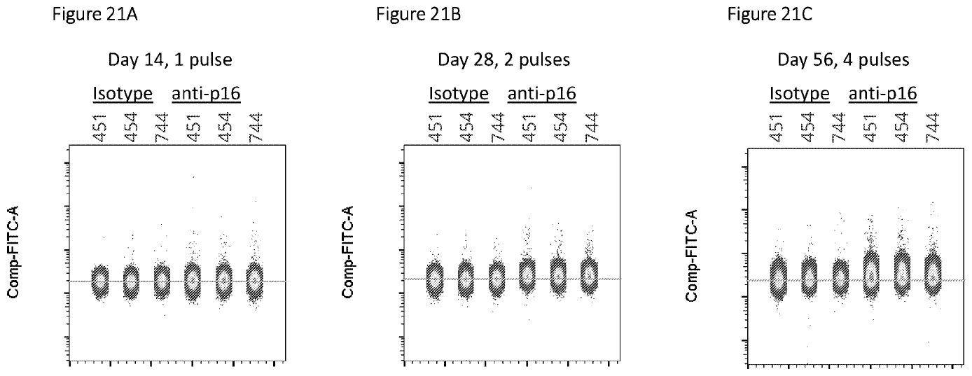

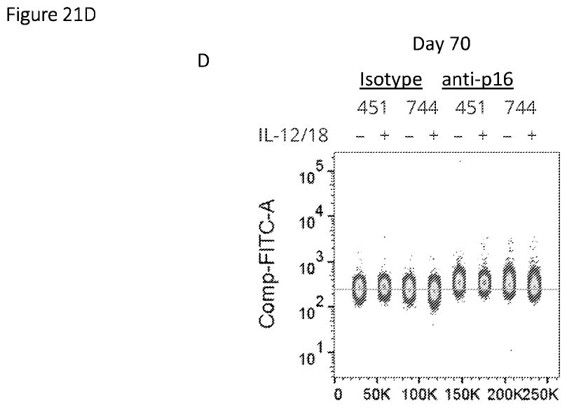

FIGS. 21A-21D trace expression p16INK4a (“p16”), which is known as a marker of aging in certain immune cells, particularly T cells and NK cells. Expression of p16 was relatively constant in the expanded NK cells at day 14, 28 and 56 of expansion, whether or not IL12/IL18 were present in the media. A subtle decrease in p16 expression was detected at day 70 of expansion with the inclusion of IL12/IL18 in the media at the pulse prior to this timepoint. This indicates that, according to several embodiments, the cells pulsed with IL12/IL18 at the final pulse of expansion do not appear to have reached a terminal expansion limit (such a limit would be associated with high p16 expression) as is seen when expanding cells that have reached senescence.

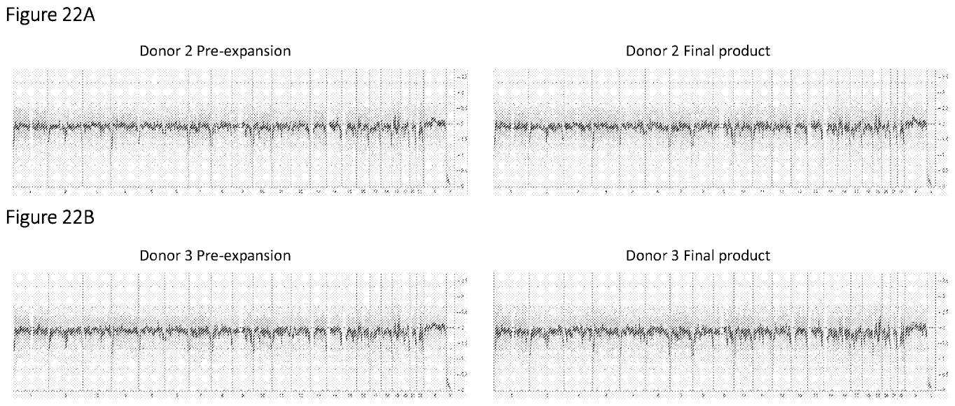

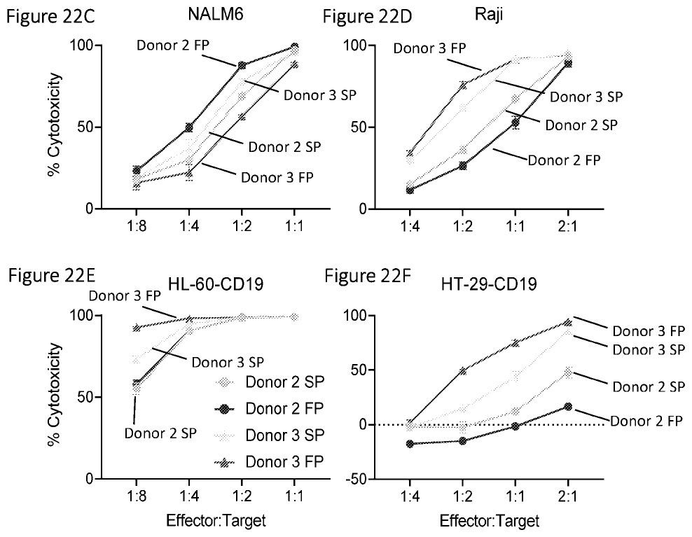

FIGS. 22A-22F show further data around the cells expanded from Donor 2 and 3. With such significant levels of expansion, there is concern that genetic abnormalities could be generated (e.g., aging cells have reduced telomeric length and therefore are subject to potential mutation and/or less effective DNA repair mechanisms (not because they are less effective on a division to division basis, but because there are so many divisions with this degree of expansion)). FIGS. 22A and 22B show chromosomal analysis across 150 single nucleotide polymorphisms from Donor 2 pre- and post-expansion (22A) and Donor 3 pre- and post-expansion (22B). The X-axis is the chromosome number and Y-axis indicates copy number. As can be seen, there is relatively consistent copy number for both donors at each of the SNPs evaluated, indicating that the cells from these Donors are genetically stable over time, despite the significant expansion. Thus, in several embodiments, donor cells exhibiting genetic stability reduced the risk of, for example, the expanding/expanded cells becoming cancerous themselves.

FIGS. 22C-22F show data related to the maintained cytotoxicity of extensively expanded cells from Donors 2 and 3 against multiple tumor cell types. Standard Process (SP) and Final Product (FP, according to methods disclosed herein) NK cells from Donor 2 and Donor 3 (and engineered to express an anti-CD19 CAR and mbIL15) were used in a cytotoxicity assay against B cell tumor cell lines that naturally express CD19 (NALM6 and Raji) and non-B cell tumor cell lines that ectopically express CD19 (HL-60-CD19 and HT-29-CD19). Percent cytotoxicity was calculated based on Incucyte images collected at 72-hours after co-culture. % cytotoxicity= [(control-experiment)/control]×100. As can be seen in each panel, depending on E:T ratio, extensively expanded NK cells exhibit cytotoxic effects against both tumor cells naturally expressing CD19 and those expressing CD19 ectopically. A notable trend in the data is a fairly close correlation between the cytotoxicity of the SP and FP cells within a given donor, indicating that while the FP cells have been far more extensively expanded, this greater cell number does not come at the expense of cytotoxicity. Thus, according to some embodiments, the methods disclosed herein generate NK cells that are about, if not more, potent than SP-expanded NK cells, and those cells are generated in a significantly greater quantity, shifting the manufacture of NK cells in an off-the-shelf allogeneic format from a future desirable goal, to an accomplished manufacturing process.

### Example 4—Evaluation of Expansion and Cytotoxicity of NK Cells from Cord Blood and Peripheral Blood

In order to further investigate the capacity for expansion of immune cells, such as NK cells, further experiments were performed to compare immune cells derived from cord blood (CB) samples or peripheral blood (PB) samples. Cells were expanded according to the methods disclosed herein, which are briefly summarized below (see also, FIG. 13). A first period of expansion is performed, which comprises between 25-35 days of expansion (e.g., ~28 days) and comprises two co-culturings (e.g., “pulses”) of cells being expanded with feeder cells. As discussed herein, the feeder cells, according to several embodiments, comprise cells that express mbIL15, 41BBL and are optionally low-expressing or devoid of MHCI (such as K562 cells). Soluble IL12 and/or soluble IL18 are used to supplement the culture media for at least one of the co-culturings in this first period. Cells are optionally frozen after this first period. Additionally, as performed here, cells can be characterized (e.g., phenotype or cytotoxicity evaluated, among other features), which can optionally serve as a gating event for the remaining expansions (e.g., if cells do not demonstrate desired characteristics after this phase, they need not be further expanded). A second expansion phase comprises between 25-35 days of expansion (e.g., ~28 days) and comprises two co-culturings (e.g., “pulses”) of cells being expanded with feeder cells. As after the first expansion, cells can optionally be evaluated at this stage as well. A third expansion phase is performed, which comprises 12-15 days (e.g., ~14 days) and comprises a single “pulse” with the feeder cells as well as media supplementation with soluble IL12 and/or IL18. In total, the expansion process comprises, in several embodiments, three phases that span ~70 days. IL2 is optionally used at one or more of the co-culturings, with concentrations ranging from about 40 to about 500 U/mL. As disclosed herein, cells may also be genetically edited (e.g., to reduce or knockout expression of a target gene/protein) and/or engineered to express, for example a CAR targeting a tumor marker of interest.

NK cells were obtained from peripheral blood of a donor and from three individual cord blood samples. Each set of cells was expanded according to embodiments disclosed herein. The PB NK and CB NK cells were co-cultured (at Day 0) with K562-mbIL15-41BBL feeder cells (at a 1:3 NK:feeder cell ratio) in growth media supplemented with IL12, IL18, and IL2. In several embodiments, the IL18 is present in the supplemented media between about 10 ng/mL and about 30 ng/ml and the IL12 is present in the supplemented media between about 0.01 ng/ml and about 10 ng/ml. In several embodiments, the media is supplemented with between about 25 and about 50 U/ml of IL2. After ~4 days, the media was supplemented again with IL2 at an elevated concentration. In several embodiments, the elevated concentration ranges from about 300 to about 500 U/mL. At Day 5, both PB NK and CB NK cells were transduced with a non-limiting embodiment of a CD19-directed CAR and mbIL15 (or mock transduced). Transduction was at a multiplicity of infection of 1.5, as a non-limiting embodiment. The media was again supplemented with an elevated concentration of IL2. The expanding PB NK or CB NK cells were pulsed again fresh feeder cells (at Day 7) and using media supplemented with IL2 at the lower concentration. The PB NK and CB NK cells were co-cultured for another 14 days before being assayed and/or cryopreserved. However, in several embodiments, the cells need not be cryopreserved, but can proceed directly to the next phase of expansion.

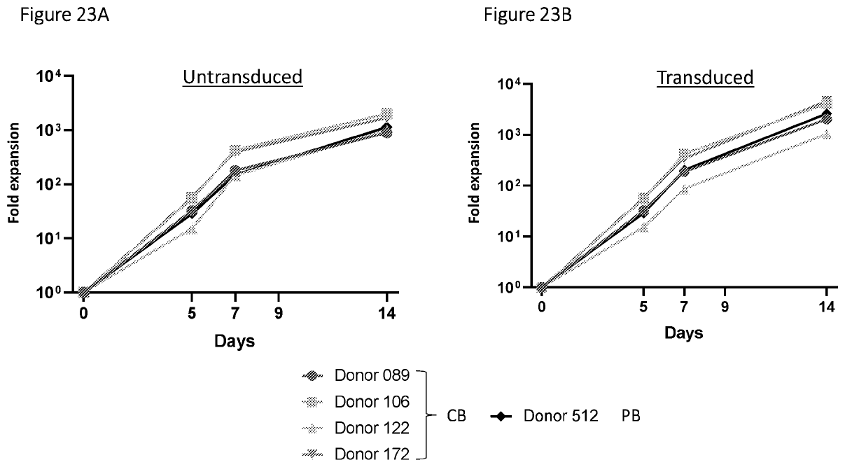

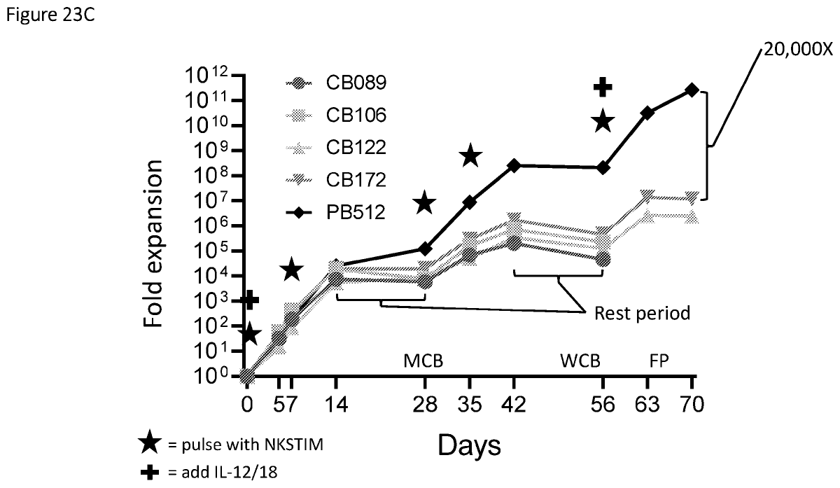

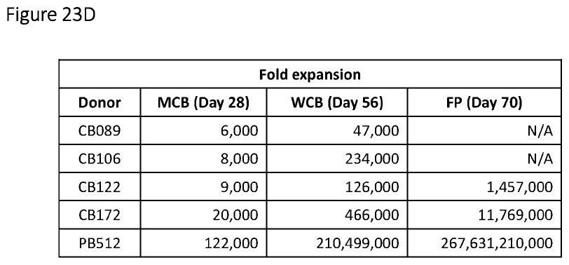

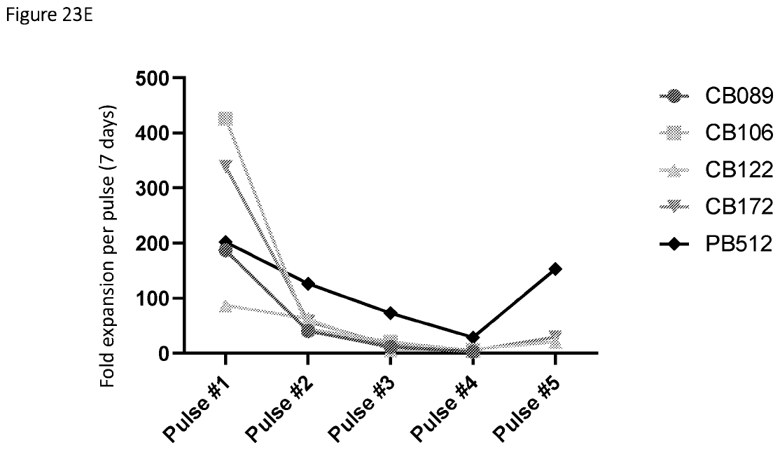

FIGS. 23A and 23B show data collected after the first expansion. FIG. 23A shows the fold expansion of mock transduced PB NK or CB NK cells. These data show that NK cells from either source are responsive to the expansion process over the first 14 days, with neither cell type showing a clearly enhanced expansion potential. FIG. 23B shows corresponding data from those cells transduced with the CD19-directed CAR. These data show, in accordance with several embodiments, that the expression of a tumor directed CAR does not appear to significantly dampen the expansion potential of NK cells either from cord blood or from peripheral blood.

Moving to expansion phase 2, frozen cells were thawed and subjected to an additional co-culture (third pulse, now at Day 28 of the process or Day 0 of phase 2). At this stage the expanded PB NK or CB NK cells were cultured with fresh feeder cells in media supplemented with low concentration of IL2. Seven days later (Day 35 overall, Day 7 of phase 2) the PB NK and CB NK cells were pulsed again with fresh feeder cells (pulse number 4). The PB NK and CB NK cells were cultured for approximately 21 days (through Day 56 overall, Day 28 of phase 2). The cells were cryopreserved at the end of that co-culturing (with a set of cells separated for phenotyping). However, in several embodiments, the cells need not be cryopreserved, but can proceed directly to the next phase of expansion.

The final expansion phase (phase 3) involves thawing the cells from the prior phase (or directly proceeding with cells that were not cryopreserved). The PB NK or CB NK cells were co-cultured with the feeder cells (pulse #5) using IL12/IL18 supplemented culture media, which was also supplemented with the lower concentration of IL2. The NK cells were co-cultured for approximately 14 days (totaling Day 70 in the overall process, Day 14 of phase 3). A portion of the cells was separated for phenotyping, while the remainder were cryopreserved. PB NK cells from the PB donor were assessed following expansion for chromosomal aberrations by KaryoStat™ analysis. 150,000 single nucleotide polymorphisms (SNPs) spread across the genome were analyzed and compared between donor matched pre- and post-expansion cells. No chromosomal aberrations were observed following expansion.

According to some embodiments, the cryopreserved cells are frozen in suitable for thawing and administration to a patient. In some embodiments, a subset of cells may be administered to a patient without being cryopreserved.

FIG. 23C shows data tracing the expansion of PB NK and CB NK cells across the three phases of expansion (5 pulses of feeder cells). Each pulse is indicated with a “star” symbol and each supplementation of the media with IL12/IL18 is indicated with a “plus” symbol. The phases are indicated above the X axis (MCB=Master Cell Bank, or phase 1; WCB=Working Cell Bank, or phase 2; FP=Final Product, or phase 3). The expansion curves for each of the sets of cells (four CB samples, and one PB sample) show that robust expansion of all cell samples occurred during phase 1. While all CB NK cells experienced modest expansion across phase 2, reaching between about 105 and about 10⁶-fold expansion, two of the CB NK cell samples continued to expand through phase 3 reaching a final expansion in this experiment of about 10⁶-10⁷-fold. PB NK cells showed a similar expansion as CB NK cells through the initial portion of phase 1, but by the end of phase 1, PB NK cells appeared to expand to a greater degree, reaching about 10⁵-fold expansion. Thereafter, the PB NK cells showed greater expansion throughout the remainder of the expansion process, with the final expanded cell population being approximately 20,000 times that of the CB NK cells. FIG. 23D tabulates the expansion data of FIG. 23C. FIG. 23E shows a breakdown of the expansion on a “per-pulse” basis. These data reflect that the initial pulse provides significant expansion for all cells (regardless of cell type), but the later phases appear to induce lesser degrees of expansion in CB NK cells, as compared to PB NK cells, which had an additional significant expansion at the fifth pulse, reaching nearly the same degree of expansion as pulse 1. Taken together, these data show that cell expansion methods as provided for herein result in substantial expansion of cell populations. In several embodiments, this allows for production of immune cell populations in quantities that are clinically relevant for a plurality of patients. These data also show that cells engineered to express a CAR, and optionally that are gene edited to reduce expression of one or more target genes/proteins, are amenable to expansion. These data also suggest that, in several embodiments, use of a peripheral blood sample is preferred as starting materials for NK cell expansion, though, as shown, cord blood samples still yielded significant expansion.

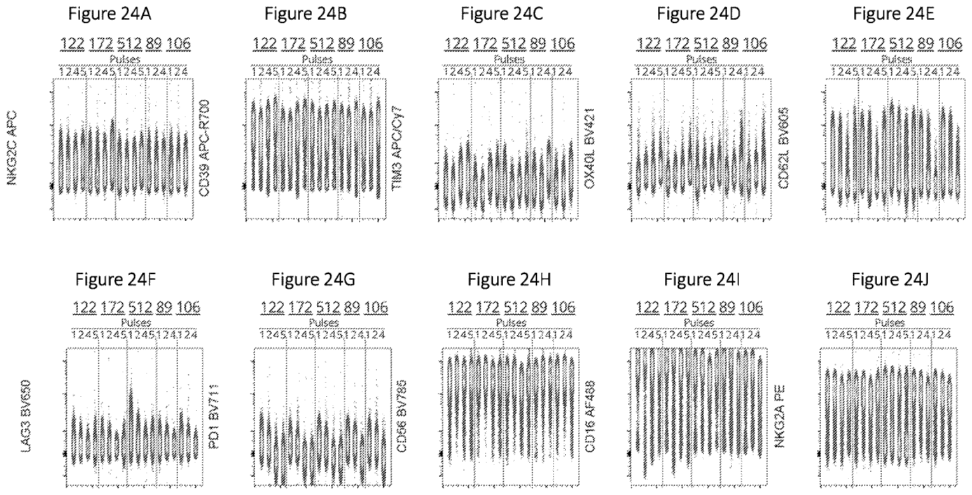

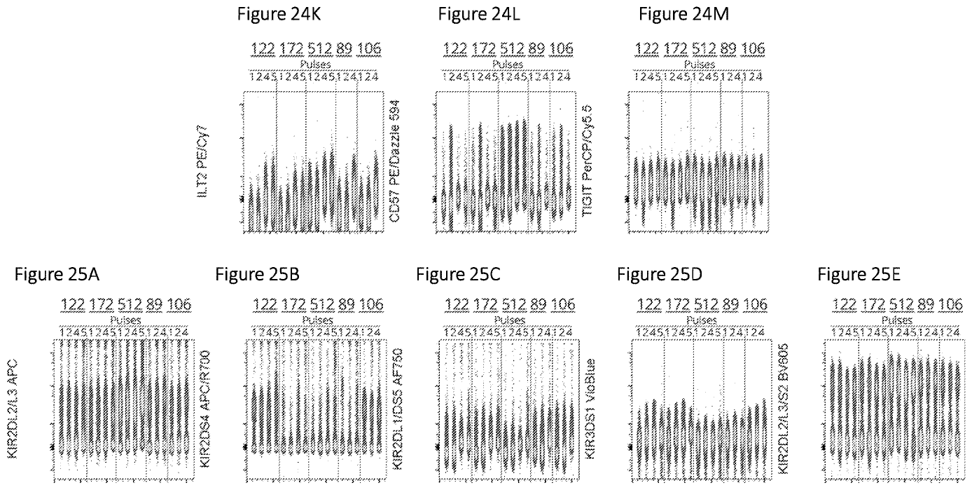

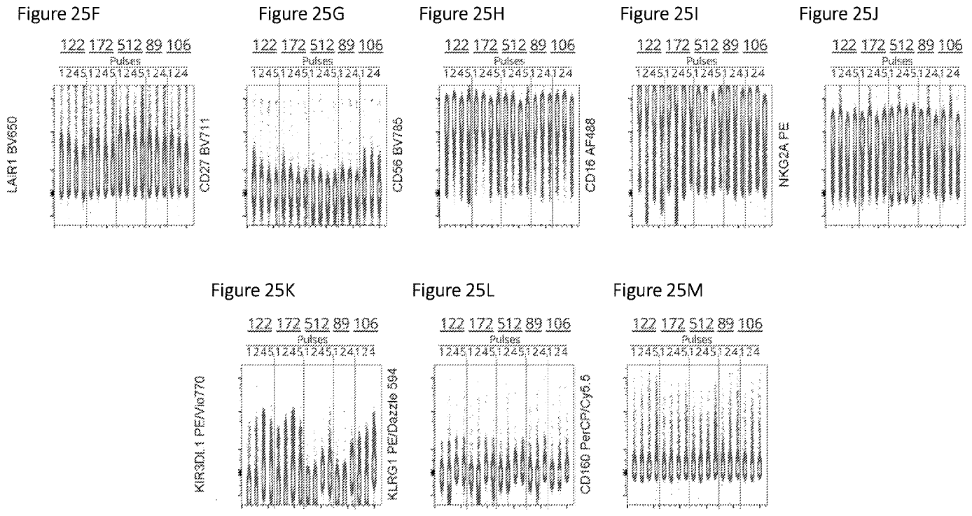

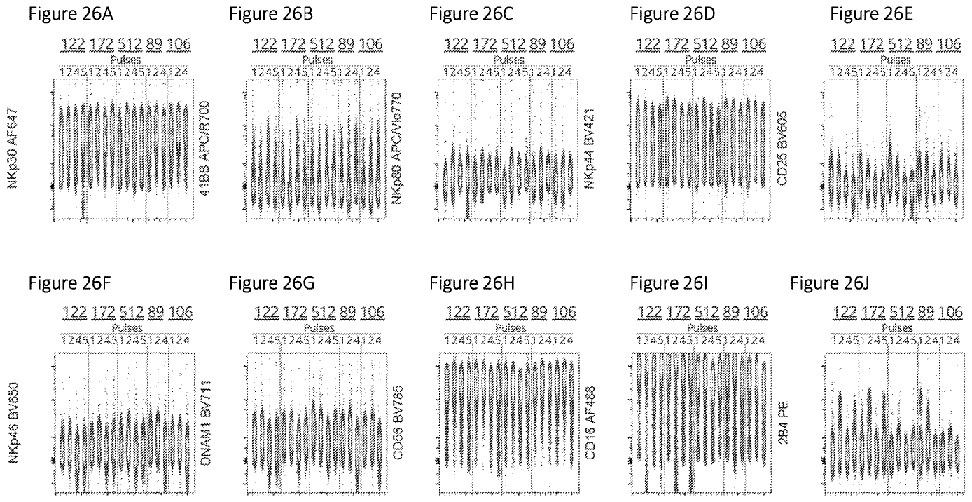

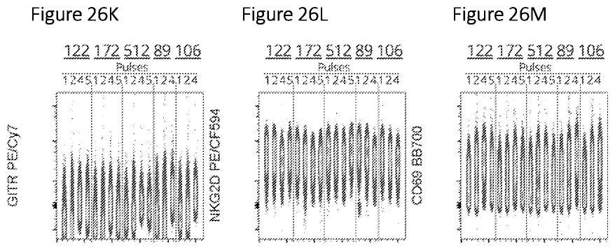

In order to attempt to correlate the degree of expansion with characteristics of the cells from the CB or PB donors, expression of various markers were assessed during the expansion process. These data are shown in FIGS. 24A-26M. Expression was measured at Days 14, 28, 56 and 70. The first panel of markers evaluated (shown in FIGS. 24A-24M) included NKG2C, CD39, TIM-3, OX40L, CD62L, LAG3, PD1, CD56, CD16, NKG2A, ILT2, CD57, and TIGIT. The second panel of markers evaluated (shown in FIGS. 25A-25M) included KIR2DL2/L3, KIR2DS4, KIR2DL1/DS5, KIR3DS1, KIR2DL2/L3/S2, LAIR1, CD27, CD56, CD16, NKG2A, KIR3DL1, KLRG1, and CD160. The third panel of markers evaluated (shown in FIGS. 26A-26M) included NKp30, 41BB, NKp80, NKp44, CD25, NKp46, DNAM1, CD56, CD16, 2B4, GITR, NKG2D, and CD69.

Additional longitudinal characterization of cell surface marker expression by flow cytometry and FlowSOM analysis was carried out at Days 14, 28, 56, and 70. The major population of expanded cells from the PB donor were characterized as NKG2C+, NKG2A−, and NKp30−, phenotypically consistent with adaptive NK cells (data not shown).

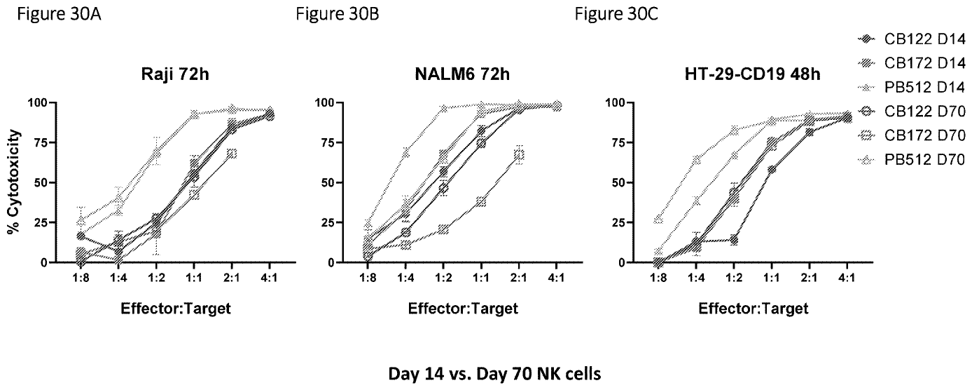

NKG2A is the first HLA class I specific inhibitory receptor to be expressed during NK cell differentiation. During an intermediate differentiation stage, NKG2A may be co-expressed with KIRs. At late stages, NKG2A expression is lost, whereas KIRs expression is maintained. In the first panel of markers assayed, most appeared to be relatively constant across the pulses and between CB and PB NK cells. However, as shown in FIG. 24J, in the PB and not the CB NK cells, NKG2A expression was lost by Day 70, correlating with a later NK differentiation stage. NKG2A is an inhibitory surface receptor on NK cells and reducing NKG2A expression may reduce an inhibitory signaling cascade and allow for the maintained anti-tumor potency as shown in FIGS. 30A-30C.

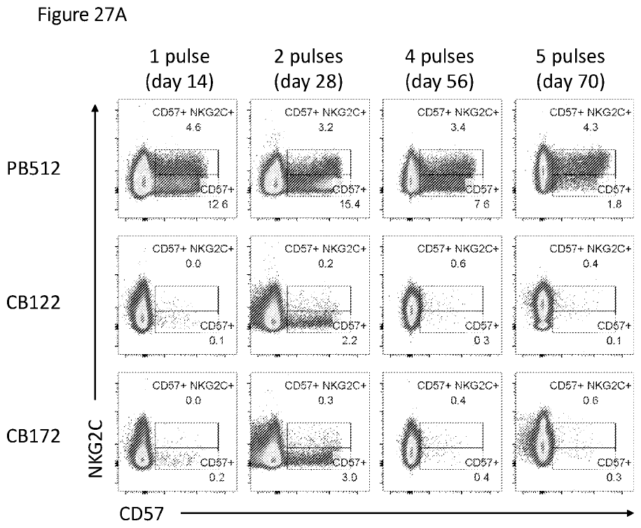

The second panel of markers, in accordance with disclosure herein related to various KIRs and their ratios being assessed to identify promising donors, evaluated several sets of KIR inhibitory receptors. As shown in FIG. 25A, the expression of the inhibitory KIR2DL2/L3 inhibitory receptor appeared to be elevated in PB NK cells as compared to CB NK cells, but relatively constant across the pulses during expansion, with a slight increase from 54% at Day 14 after 1 pulse, to 90% at Day 70 with 5 pulses (Percentages shown in FIG. 27B).

In marker panel 3, FIG. 26C, most of the markers remain consistent except for an increase in CD69 expression. As shown in FIG. 26M, CD69 expression appears to be elevated with pulse number. CD69 expression is increased after NK cells are stimulation with IL2, so this increase is not unexpected. CD69 expression increase may also reflect its function as a costimulatory molecule during expansion or sustaining NK cell activation (as has been seen in T cells).

Taken together, these marker screens indicate that the final expanded CB NK cells were CD57⁻KIR^{lo/-} and NKG2A⁺, consistent with an immature phenotype. The final expanded PB NK cells were educated, at more than 80% NKG2A⁻ KIR⁺. Moreover, the data from these expression panels suggest that the expansion process may not only increase the cell number, but also alter the expanded cells activation and/or persistence.

To further investigate the expansion and activation capacity of CB NK and PB NK cells, more specific screening was performed. As shown in FIG. 27A, expression of the activating NKG2C receptor and a marker of NK cell maturation, CD57, was evaluated. In general, increased expression of NKG2C and CD57 would be indicative of differentiation of NK cells (more expression means more differentiated), eventually taking on an adaptive-like phenotype. Interestingly, the two CB NK cell samples studied expressed very little CD57 at any time point during expansion. Additionally, the PB NK cell populations expressing little to no NKG2C appears to also show a reduction in percentage over time (dropping from ~13% to about 2% over the 70-day expansion process). CD57 is typically used to identify terminally differentiated cells with reduced proliferative capacity, so the reduced expression of CD57 may represent a reversal of that status to a state with additional proliferation potential.

As discussed herein, the KIR profile of a given donor cell may impact the overall expansion and/or activity capacity of NK cells expanded from that donor. NK cells that are “educated” (prior interaction of inhibitory KIRs with MHC ligands) have been shown to be hyperresponsive to stimulation. KIR educated cells must also have expression of the KIRs on their cell surface. Given that CB cells are relatively young, their KIR expression is lower than that of PB cells. The expression of NKG2A (inhibitory) was evaluated along with KIR2DL2/3 expression. FIG. 27B shows the resultant data. This Figure shows demonstrates that there is a preferential expansion of KIR educated PB NK cells which is also associated with a reduced level of NKG2A expression. As seen in FIG. 27B, at day 14 the percentage of cells expressing low KIR2DL2/3 and high NKG2A was about 40% (see Q1) while those expressing low NKG2A and higher KIR2DL2/3 was about 24% (see Q4). The expansion process resulted in a “shift” of cells from Q1 to Q4 over the 70 days, as shown in the decrease to ~7% cells in Q1 at Day 70 (high NKG2A/low KIR) and the increase to ~85% of cells in Q4 at Day 70 (low NKG2A/high KIR). Thus, enrichment of NKG2A−KIR+ educated NK cells was observed for NK cells expanded from peripheral blood, but not from NK cells expanded from cord blood. These data suggest that, in several embodiments, use of a KIR educated (e.g., peripheral blood) starting NK cell population may enhance the expansion potential.

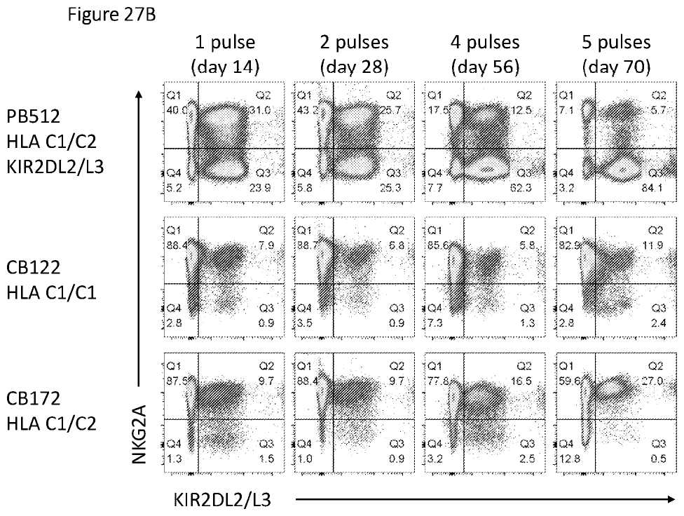

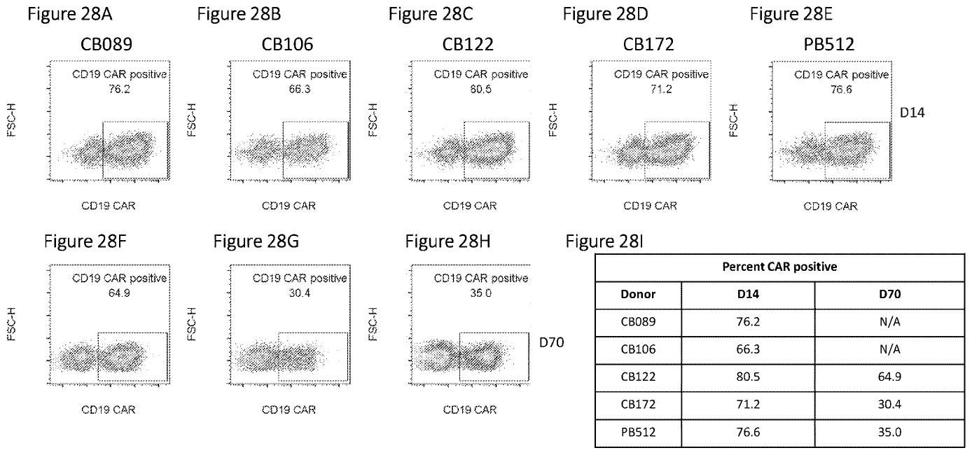

While robust expansion of NK cells using the methods disclosed herein is an important aim, maintaining the expression of CAR constructs that those cells have been engineered to express, such that the resulting expanded population maintains the desired engineered-in cytotoxicity, is also a central objective. As mentioned above, PB NK and CB NK cells were transduced with, as a non-limiting example, a CD19-directed CAR. In this example, opposed to Example 3 above, in which the NK cells were engineered to express the CAR and also edited to reduce expression of CISH, the PB NK and CB NK cells were not gene edited. FIGS. 28A-28H show CD19 expression data at Day 14 of expansion (top row) and at Day 70 (bottom row). It is notable in this evaluation that both CB NK cells (compare 28C to 28F and 28D to 28G) and PB NK cells (compare 28E to 28H) exhibited decreases in the expression of the CD19 CAR over the course of expansion. FIG. 28I tabulates the expression data. As compared to, for example, FIG. 17A, in which PB NK cells edited for CISH maintained CD19 CAR expression, here, the absence of CISH led to a reduction in expression of the CAR. As will be discussed below, the reduced expression still allowed for cytotoxicity, however, in several embodiments, dis-inhibiting the positive impact of mbIL15 expression (which is encoded by, though separately expressed, the polynucleotide encoding the CD19 CAR construct) by genetically reducing levels of CISH, results in maintained (or less reduction) in CD19 CAR expression. In several embodiments, such edits result in further enhanced cytotoxicity.

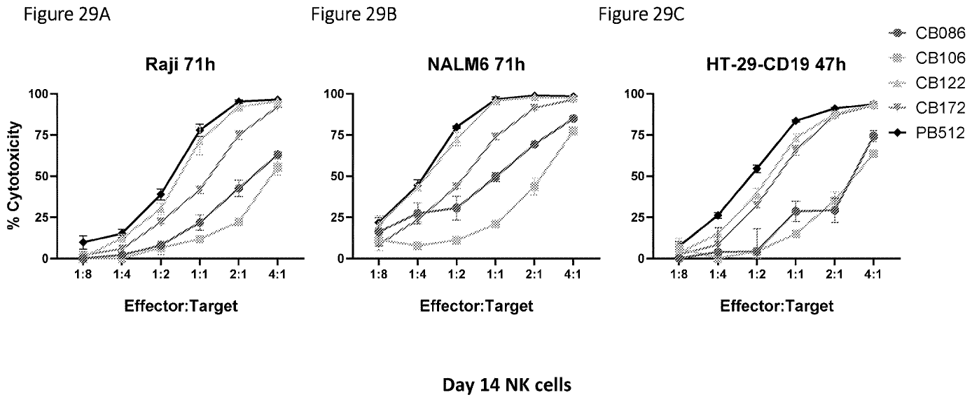

FIGS. 29A-29C show cytotoxicity data of PB NK and CB NK cells (while still expressing higher levels of the CD19 CAR (as shown in FIG. 28, top row) at Day 14 of expansion. Cytotoxicity was evaluated against Raji cells (Burkitt lymphoma), NALM6 cells (B cell precursor leukemia), and HT-29-CD19 (a colorectal adenocarcinoma engineered to ectopically express CD19). Percent cytotoxicity was calculated based on Incucyte images (representing fluorescence intensity of target tumor cells) collected at the indicated timepoint after co-culture and the indicated E:T ratio. As shown in the figures, at least with these three non-limiting embodiments of target tumor cell lines, the PB NK cells expressing the CD19 CAR appeared to have a greater cytotoxicity against the target cells in comparison to the CB NK cells, though the CB NK cells from donor 122 (upright triangle) performed quite similarly against both the RAJI and NALM6 cells lines. CB donor 172 (inverted triangles) showed reduced cytotoxicity and CB donors 086 and 106 showed the least cytotoxicity, though still able to achieve ~60-70% cytotoxicity at a 4:1 E:T ratio. It is noted that these two donors also did not complete the full 70 day expansion at therefore may represent NK cell samples that are otherwise less robust (in terms of overall cell health, impacting both expansion capacity and cytotoxicity) since they did express similar levels of the CAR (see FIG. 28I).

Despite the reduced expression of the CD19 CAR observed at 70 days discussed above, the expanded NK cells still retained substantial cytotoxicity against target tumor cells, as shown in FIGS. 30A-30C. Cytotoxicity was evaluated against the same three cell lines as in FIGS. 29A-29C but comparing Day 14 cells versus Day 70. Day 14 data is shown with filled shapes and Day 70 is shown in open shapes. With Raji cells as the target, PB NK cells (triangles) showed little to no difference in terms of cytotoxicity generated by Day 14 versus Day 70 cells. Similar results are shown for CB NK cells from donor 122 (circles), though some decreased cytotoxicity was observed for CB NK cells from donor 172 (squares). Cytotoxicity was somewhat more distinct for Day 14 versus Day 70 cells when targeting NALM6 cells. With each sample, the Day 14 cells appeared to show modestly higher cytotoxicity, although again, it should be noted that at a 1:1 E:T ratio 5 of 6 of the experimental groups achieved or exceeded ~75% cytotoxicity. In contrast, against the ectopic CD19 expressing HT-29-CD19 cells, the PB NK cells and CB NK cells from donor 122 appeared more potent after 70 days, despite the reduced CD19 CAR expression. CB NK cells from donor 172 performed substantially similar to one another. Notable again is, at 2:1 E:T, all cell samples achieved or exceeded 75% cytotoxicity (5 of 6 met this performance level at 1:1 E:T). In several embodiments, the positive activating and persistence effects of the expansion processes disclosed herein are sufficient to offset a reduced expression of the cancer targeting CAR that may occur, though it can be obviated, in several embodiments, by gene editing of, for example CISH. Taken together, these data demonstrate that even profoundly expanded cell populations retain significant cytotoxicity against target cells. With the PB NK cells, potency is maintained even after a 250 billion-fold expansion. Thus, according to several embodiments disclosed herein, expansion of immune cells, such as NK cells, that are engineered to express a tumor targeting CAR (and/or edited at one or more gene targets) yields substantial increases in cell populations, and maintenance of significant cytotoxic potential against target tumor cells.

### Example 5—Additional Evaluation of Expansion and Cytotoxicity of NK Cells from Peripheral Blood

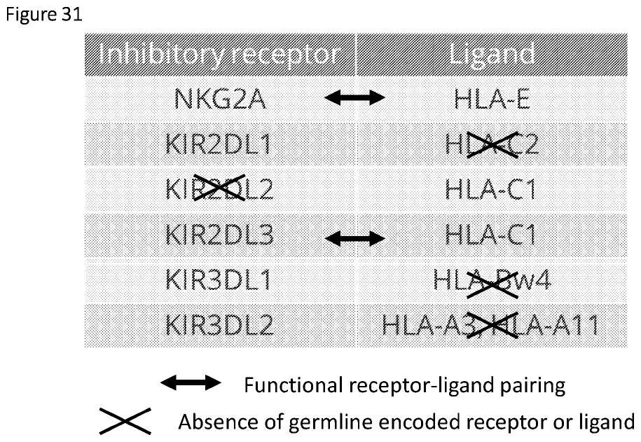

Natural killer cells derived from a peripheral blood donor sample (PB NK cells) were assessed for their capacity for expansion and cytotoxicity. The donor was observed to express the inhibitory KIRs NKG2A, KIR2DL1, KIR2DL3, KIR3DL1, and KIR3DL2, as well as ligands HLA-E and HLA-C1 (FIG. 31). Accordingly, for this donor, NK cells could be KIR-educated by virtue of the interaction of KIR2DL3 with the HLA-C1 ligand.

PB NK cells were expanded for 14 days in the presence of IL-2, IL-12, IL-18, and K562 cells modified to express mbIL15 and 4-1BBL (“NKSTIM”). In some cases, the PB NK cells were also genetically modified to express an anti-CD19 CAR-mbIL15 construct as described in Example 1.

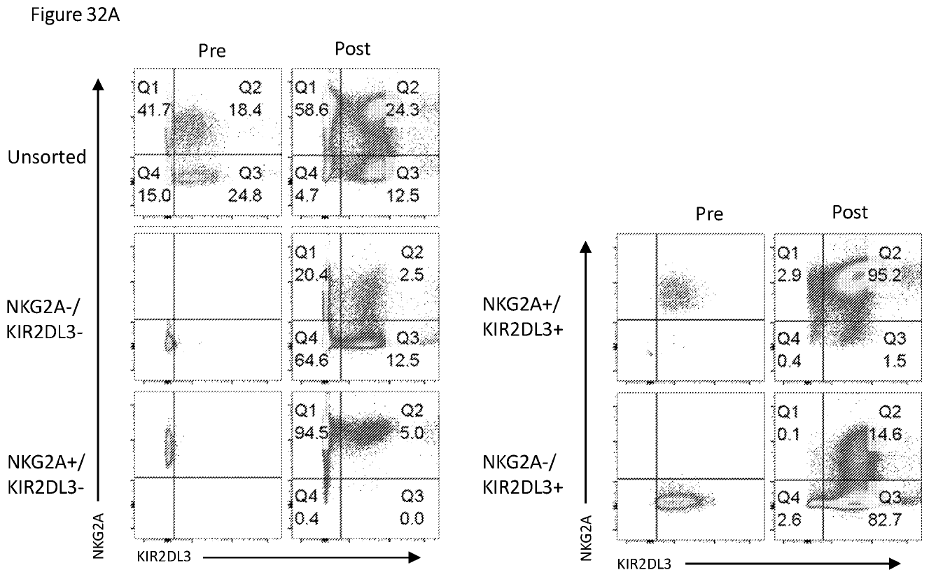

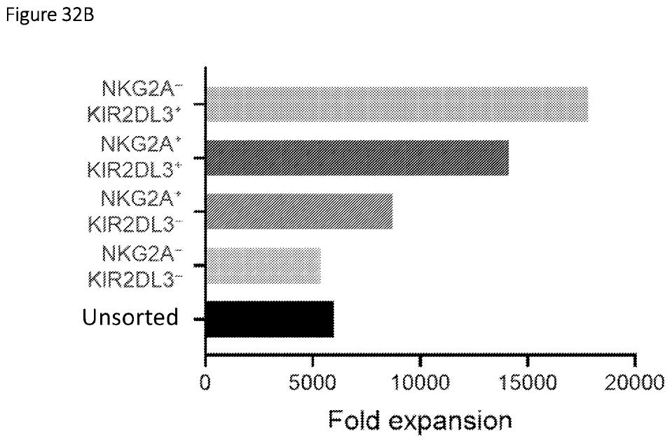

PB NK cells were sorted for NKG2A, KIR2DL3, and CD57 expression prior to expansion (“Pre”), and expression was re-analyzed following the 14-day expansion (“Post”). As shown in FIG. 32A and quantified in FIG. 32B, NKG2A−/KIR2DL3+ PB NK cells exhibited the greatest expansion, followed by NKG2A+/KIR2DL3+ cells. Data for CD57+ cells are shown, though similar results were obtained for CD57− cells. The increase in expansion among educated NK cells (KIR2DL3+ NK cells for this donor) may explain the enrichment of KIR-educated PB NK cells over 70 days of culture, as described in Example 4.

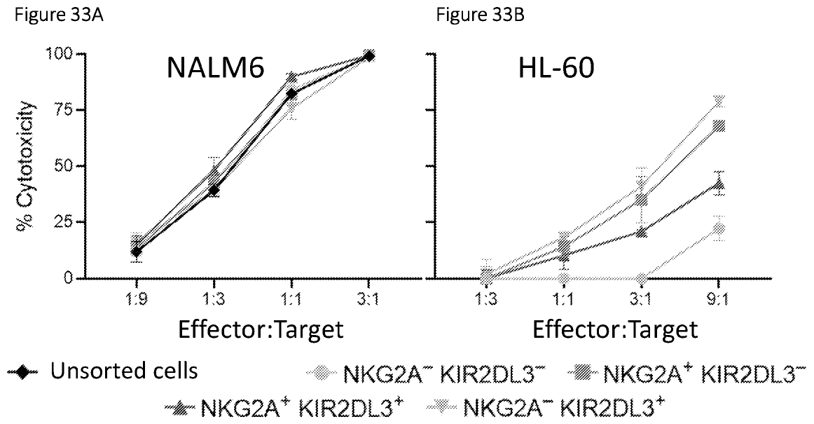

Transduced PB NK population subsets were incubated with Nalm6 cells expressing CD19 (FIG. 33A), and untransduced PB NK population subsets were incubated with HL-60 cells not expressing CD19 (FIG. 33B), to assess CAR-driven cytotoxicity and natural cytotoxicity, respectively. The PB NK cells were incubated with the target cells at various E:T ratios for 4 hours. The transduced PB NK population subsets all demonstrated similar cytotoxicity against CD19-expressing NALM6 cells, while differences in cytotoxicity were observed among the untransduced PB NK population subsets against HL-60 cells not expressing CD19. These data are consistent with a finding that CAR-driven cytotoxicity is comparable among educated and uneducated NK cells, whereas natural cytotoxicity may be greater for educated NK cells.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “administering a population of expanded NK cells” includes “instructing the administration of a population of expanded NK cells.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “90%” includes “90%.” In some embodiments, at sequence having at least 95% sequence identity with a reference sequence includes sequences having 96%, 97%, 98%, 99%, or 100% identical to the reference sequence. In addition, when a sequence is disclosed as “comprising” a nucleotide or amino acid sequence, such a reference shall also include, unless otherwise indicated, that the sequence “comprises”, “consists of” or “consists essentially of” the recited sequence.

Articles such as “a”, “an”, “the” and the like, may mean one or more than one unless indicated to the contrary or otherwise evident from the context. The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, amount, dose, administration route, cell type, target, cellular marker, antigen, targeting moiety, or combination thereof.

In several embodiments, there are provided amino acid sequences that correspond to any of the nucleic acids disclosed herein, while accounting for degeneracy of the nucleic acid code. Furthermore, those sequences (whether nucleic acid or amino acid) that vary from those expressly disclosed herein, but have functional similarity or equivalency are also contemplated within the scope of the present disclosure. The foregoing includes mutants, truncations, substitutions, or other types of modifications.

Any titles or subheadings used herein are for organization purposes and should not be used to limit the scope of embodiments disclosed herein.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### Sequences

In several embodiments, there are provided amino acid sequences that correspond to any of the nucleic acids disclosed herein (and/or included in the accompanying sequence listing), while accounting for degeneracy of the nucleic acid code. Furthermore, those sequences (whether nucleic acid or amino acid) that vary from those expressly disclosed herein (and/or included in the accompanying sequence listing), but have functional similarity or equivalency are also contemplated within the scope of the present disclosure. The foregoing includes mutants, truncations, substitutions, codon optimization, or other types of modifications.

In accordance with some embodiments described herein, any of the sequences may be used, or a truncated or mutated form of any of the sequences disclosed herein (and/or included in the accompanying sequence listing) may be used and in any combination.

A Sequence Listing in electronic format may be submitted herewith. Some of the sequences provided in the Sequence Listing may be designated as Artificial Sequences by virtue of being non-naturally occurring fragments or portions of other sequences, including naturally occurring sequences. Some of the sequences provided in the Sequence Listing may be designated as Artificial Sequences by virtue of being combinations of sequences from different origins, such as humanized antibody sequences. Appendix A is attached and incorporated by reference herein and provides sequence information for nucleic acid and amino acid sequences provided for herein.

What is claimed is:

1. A method of enriching for educated natural killer (NK) cells from a biological sample, comprising, from a biological sample comprising NK cells, enriching for cells that express an inhibitory Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+),
the enriching thereby generating an enriched population comprising educated NK cells.

2. The method of claim 1, wherein the enriching comprises enriching for cells that both (i) express an iKIR selected from among the group consisting of: KIR2DL1, KIR2DL2, KIR2DL3, KIR3DL1, KIR3DL2, KIR3DL3, KIR2DL5, and any combination thereof (iKIR+); and (ii) do not express NKG2A (NKG2A−).

3. The method of claim 1, wherein at least about 25%, at least about 30%, at least about 35%, at least about 40%, at least about 45%, or at least about 50% of cells in the enriched population comprise NK cells that express an iKIR (iKIR+ cells).

4. The method of claim 1, wherein at least about 10%, at least about 15%, at least about 20%, at least about 25%, or at least about 30% of cells in the enriched population comprise NK cells that both express an iKIR and do not express NKG2A (iKIR+NKG2A− cells).

5. The method of claim 1, wherein enriching for cells that express an iKIR comprises enriching for cells that express one iKIR, two iKIRs, or three iKIRs.

6. The method of claim 1, wherein the enriching comprises enriching for cells that express an iKIR comprises positive selection.

7. The method of claim 1, wherein the enriching comprises enriching for cells that do not express NKG2A comprises negative selection.

8. The method of claim 1, wherein the enriching comprises enriching for cells that express an iKIR comprises contacting the NK cells with an antibody that binds to iKIR.

9. The method of claim 1, wherein the enriching comprises enriching for cells that do not express NKG2A comprises contacting the NK cells with an antibody that binds to NKG2A.

10. The method of claim 1, further comprising expanding the enriched population comprising educated NK cells, thereby generating an expanded and enriched NK cell population comprising educated NK cells.

11. The method of claim 10, wherein expanding the enriched population comprising educated NK cells comprises co-culturing NK cells of the enriched population with a feeder cell population, optionally in a culture media supplemented with soluble interleukin 12 (IL12) and/or soluble interleukin 18 (IL18).

12. The method of claim 11, wherein the feeder cell population comprises cells engineered to express 4-1BBL and membrane-bound interleukin-15 (mbIL 15).

13. The method of claim 11, wherein the ratio of NK cells to feeder cells ranges from about 1:2 to about 1:10, optionally from about 1:3 to about 1:5.

14. The method of claim 11, wherein: (a) the IL12 is present in the supplemented culture media at a concentration ranging from about 0.01 ng/ml to about 10 ng/ml; and/or (b) the IL18 is present in the supplemented culture media at a concentration ranging from about 10 ng/ml to about 30 ng/mL.

15. The method of claim 11, wherein the culture media is supplemented with soluble interleukin 2 (IL2).

16. The method of claim 15, wherein the IL2 is present in the supplemented media at a concentration ranging from about 25 units/mL to about 50 units/mL.

17. The method of claim 11, wherein expanding the enriched population comprising educated NK cells does not comprise contacting NK cells with a stimulator of NKG2A, NKG2C, or both.

18. The method of claim 11, wherein expanding the enriched population comprising educated NK cells does not comprise contacting NK cells with HLA-E.

19. The method of claim 11, wherein the feeder cells of the feeder cell population do not express HLA-E.

20. The method of any one of claims 1-19, further comprising obtaining the biological sample from a donor.

21. The method of any one of claims 1-20, wherein the biological sample is a blood sample.

22. The method of claim 21, wherein the blood sample is a peripheral blood sample.

23. The method of claim 21, wherein the blood sample is a cord blood sample.

24. The method of any one of claims 10-23, wherein expanding the enriched population comprising educated NK cells comprises co-culturing NK cells of the enriched population with a feeder cell population for a first time, optionally wherein expanding the enriched population comprising educated NK cells comprises co-culturing NK cells of the enriched population with a feeder cell population for a second time.

25. The method of any one of claims 10-24, wherein: (a) the method further comprises genetically editing NK cells of the expanded NK cell population or the biological sample to reduce or eliminate expression of at least one endogenous gene or protein expressed as compared to a non-modified NK cell, thereby generating a genetically edited NK cell population; and/or (b) NK cells of the expanded NK cell population or the biological sample are genetically edited to reduce or eliminate expression of at least one endogenous gene or protein expressed as compared to a non-modified NK cell, thereby generating a genetically edited expanded NK cell population comprising educated NK cells.

26. The method of any one of claims 10-25, wherein: (a) the method further comprises engineering NK cells of the expanded NK cell population or the genetically edited NK cell population to express a chimeric antigen receptor (CAR) that binds an antigen, thereby generating an engineered NK cell population; and/or (b) NK cells of the expanded NK cell population or the genetically edited NK cell population are engineered to express a chimeric antigen receptor (CAR) that binds to an antigen, thereby generating an engineered NK cell population.

27. The method of claim 26, wherein the antigen is selected from a ligand for the NKG2D receptor, CD19, CD70, BCMA, or CD38.

28. The method of claim 26 or claim 27, wherein the CAR comprises a transmembrane domain and an intracellular signaling region.

29. The method of claim 28, wherein the transmembrane domain comprises CD8, CD28, or a portion thereof, optionally wherein the transmembrane domain comprises CD8a or a portion thereof.

30. The method of claim 28 or claim 29, wherein the intracellular signaling region comprises a CD3zeta domain.

31. The method of any one of claims 28-30, wherein the intracellular signaling region comprises an intracellular signaling domain of an OX40, 4-1BB, CD28, or a signaling portion thereof, optionally wherein the intracellular signaling region comprises an intracellular signaling domain of OX40 or a signaling portion thereof.

32. The method of any one of claims 10-31, wherein: (a) the method comprises engineering NK cells of the expanded NK cell population, the genetically edited NK cell population, or the engineered NK cell population to express membrane-bound interleukin 15 (mbIL15); and/or (b) NK cells of the expanded NK cell population, the genetically edited NK cell population, or the engineered NK cell population are engineered to express membrane-bound interleukin 15 (mbIL15).

33. A method of producing a population of engineered natural killer (NK) cells, the method comprising:
(a) from a biological sample comprising NK cells, enriching for cells that express an inhibitor Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), the enriching thereby generating an enriched population comprising educated NK cells;
(b) expanding the enriched population comprising educated NK cells, thereby generating an expanded NK cell population; and
(c) engineering NK cells of the expanded NK cell population to express a chimeric antigen receptor (CAR) that binds an antigen, thereby generating an engineered NK cell population.

34. A population of NK cells produced by the method of any one of claims 1-33.

35. The population of NK cells of claim 34 for use in the treatment of a disease or condition in a subject.

36. The population of NK cells of claim 35, wherein the disease or condition is a cancer.

37. The population of NK cells of claim 35, wherein the disease or condition is an autoimmune disease.

38. Use of the population of NK cells of claim 34 for the treatment of a disease or condition in a subject.

39. Use of the population of NK cells of claim 34 in the preparation of a medicament for the treatment of a disease or condition in a subject.

40. A method of treating a disease or condition in a subject, comprising administering to the subject the population of NK cells of claim 34.

41. The population of NK cells of any one of claims 34-37, the use of claim 38 or claim 39, or the method of claim 40, wherein the population of NK cells is allogeneic to the subject.

42. The population of NK cells of claim 35 or claim 41, the use of any one of claims 38 or 39, or the method of claim 40, wherein the disease or condition is a cancer.

43. The population of NK cells of any one of claims 34-36 or claim 41 or 42, the use of claim 38 or 39, or the method of claim 40 or 41, wherein the cancer is a hematologic malignancy, optionally wherein the hematologic malignancy is a leukemia or a lymphoma.

44. The population of NK cells of any one of claims 34-36, 41-43, the use of claim 38 or 39, or the method of claim 40, wherein the CAR binds to an antigen expressed by the cancer.

45. The population of NK cells of claim 34 35, or 41, the use of any one of claims 38, 39, or the method of claim 40, wherein the disease or condition is an autoimmune disease.

46. The method, population, or use of any one of claims 1-45, wherein the biological sample is from a subject who is CMV positive (CMV+).

47. The method, population, or use of any one of claims 1-46, wherein the iKIR+ cells do not express CD57 (CD57−).

48. The method, population, or use of any one of claims 2-47, wherein the iKIR+NKG2A− cells do not express CD57 (CD57−).

49. The method, population, or use of any one of claims 1-48, wherein the iKIR+ cells do not express NKG2C (NKG2C−).

50. The method, population, or use of any one of claims 2-49, wherein the iKIR+NKG2A− cells do not express NKG2C (NKG2C−).

51. A method for selecting a donor of natural killer (NK) cells for immunotherapy, comprising
(a) assessing in a biological sample from a donor, the percentage of cells expressing an inhibitory Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), wherein the biological sample comprises NK cells;
(b) determining whether the percentage of iKIR+ cells in the biological sample is at or above a threshold value, wherein the threshold value is at least about 25%; and
(c) if the percentage of iKIR+ cells is at or above the threshold value, selecting the biological sample for use in immunotherapy; or
if the percentage of iKIR+ cells is less than the threshold value, not selecting the biological sample for use in immunotherapy.

52. The method of claim 51, wherein the threshold value is about 25%, about 30%, about 35%, about 40%, about 45%, or about 50%.

53. A method for selecting a donor of natural killer (NK) cells for immunotherapy, comprising
(a) assessing in a biological sample from a donor, the percentage of cells that both (i) express an inhibitory Killer Cell Ig-like Receptor (iKIR) selected from among the group consisting of: KIR2DL1, KIR2DL2, KIR2DL3, KIR3DL1, KIR3DL2, KIR3DL3, KIR2DL5, or any combination thereof (iKIR+); and (ii) do not express NKG2A (NKG2A−), wherein the biological sample comprises NK cells;
(b) determining whether the percentage of iKIR+NKG2A− cells in the biological sample is at or above a threshold value, wherein the threshold value is at least about 10%; and
(c) if the percentage of iKIR+NKG2A− cells is at or above the threshold value, selecting the biological sample for use in immunotherapy; or
if the percentage of iKIR+NKG2A− cells is less than the threshold value, not selecting the biological sample for use in immunotherapy.

54. The method of claim 53, wherein the threshold value is about 10%, about 15%, about 20%, about 25%, or about 30%.

55. The method of any one of claims 51-54, further comprising obtaining the biological sample from the donor.

56. The method of any one of claims 51-55, wherein the biological sample is a blood sample.

57. The method of claim 56, wherein the blood sample is a peripheral blood sample.

58. The method of claim 56, wherein the blood sample is a cord blood sample.

59. The method of any one of claims 51-58, wherein cells that express an iKIR express one iKIR, two iKIRs, or three iKIRs.

60. The method of any one of claims 51-59, wherein if the biological sample is selected for use in immunotherapy, the method further comprises expanding the NK cells of the biological sample, thereby generating an expanded NK cell population.

61. The method of claim 60, wherein expanding the NK cells of the biological sample comprises co-culturing, in a culture media supplemented with soluble interleukin 12 (IL12) and soluble interleukin 18 (IL18), NK cells of biological sample with a feeder cell population.

62. The method of claim 61, wherein the feeder cell population comprises cells engineered to express 4-1BBL and membrane-bound interleukin-15 (mbIL 15).

63. The method of any one of claims 60-62, wherein expanding the NK cells of the biological sample does not comprise contacting NK cells with a stimulator of NKG2A, NKG2C, or both.

64. The method of any one of claims 60-63, wherein expanding the NK cells of the biological sample does not comprise contacting NK cells with HLA-E.

65. The method of any one of claims 61-64, wherein the feeder cells of the feeder cell population do not express HLA-E.

66. The method of any one of claims 51-65, further comprising genetically editing NK cells of the expanded NK cell population or the biological sample to reduce or eliminate expression of at least one endogenous gene or protein expressed as compared to a non-modified NK cell, thereby generating a genetically edited NK cell population.

67. The method of any one of claims 60-66, further comprising engineering NK cells of the expanded NK cell population or the genetically edited NK cell population to express a chimeric antigen receptor (CAR) that binds a tumor antigen, thereby generating an engineered NK cell population.

68. The method of any one of claims 51-67, wherein the immunotherapy is for treating a disease or condition in a subject.

69. The method of claim 68, wherein the disease or condition is a cancer.

70. The method of claim 69, wherein the cancer is a hematologic malignancy, optionally wherein the cancer is a leukemia or a lymphoma.

71. The method of claim 69 or claim 70, wherein the CAR binds to an antigen expressed by the cancer.

72. The method of claim 68, wherein the disease or condition is an autoimmune disease.

73. The method of any one of claims 68-72, wherein the NK cells of the biological sample are allogeneic to the subject.

74. The method of any one of claims 51-73, wherein the iKIR+ cells do not express CD57 (CD57−).

75. The method of any one of claims 53-74, wherein the iKIR+NKG2A− cells do not express CD57 (CD57−).

76. The method of any one of claims 51-75, wherein the iKIR+ cells do not express NKG2C (NKG2C−).

77. The method of any one of claims 53-77, wherein the iKIR+NKG2A− cells do not express NKG2C (NKG2C−).

78. The method of any one of claims 51-78, wherein the donor is CMV positive (CMV+).

79. A method of increasing the expansion of natural killer cells, the method comprising:
from a biological sample comprising NK cells, enriching for cells that express an inhibitor Killer Cell Ig-like Receptor (iKIR) selected from among the group consisting of: KIR2DL1, KIR2DL2, KIR2DL3, KIR3DL1, KIR3DL2, KIR3DL3, KIR2DL5, and any combination thereof (iKIR+), the enriching thereby generating an enriched population comprising educated NK cells; and
expanding the enriched population comprising educated NK cells, thereby generating an expanded NK cell population.


SEQUENCE LISTING (107 sequences)

SEQ ID NO: 1 - DNA, 1797 bp, Synthetic construct
  source 1..1797: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 2 - protein, 599 aa, Synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 3 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 4 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 5 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 6 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 7 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggattggcgtgatttggggcagcgaaaccacctattataacagcgcgctgaaaagccgcctgaccattagcaaagataacagcaaaaaccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 8 - protein, 706 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 9 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcctgacctgcaccgtgagcggcgtgagcctgccggattatggcgtgagctggattcgccagccgccgggcaaaggcctggaatggctgggcgtgatttggggcagcgaaaccacctattataacagcgcgctgaaaagccgcctgaccattagcaaagataacagcaaaagccaggtgagcctgaaactgagcagcgtgaccgcggcggataccgcggtgtattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 10 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 11 - DNA, 2153 bp, Synthetic construct
  source 1..2153: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcagatctagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 12 - protein, 707 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 13 - DNA, 2150 bp, Synthetic construct
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccctgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 14 - protein, 706 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 15 - DNA, 2150 bp, Synthetic construct
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttactactgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 16 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 17 - DNA, 2150 bp, Synthetic construct
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccaggtggaactgttaaactcctgatctaccatacatcaagattacactcaggagtcccatcaaggttcagtggcagtgggtctggaacagatttcaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 18 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 19 - DNA, 2150 bp, Synthetic construct
  source 1..2150: mol_type = other DNA
gaattcgccgccaccatggccttaccagtgaccgccttgctcctgccgctggccttgctgctccacgccgccaggccggacatccagatgacacagagcccgtcctccctgtctgcctctgtgggagacagagtcaccatcacctgcagggcaagtcaggacattagtaaatatttaaattggtatcagcagaaaccagacggaactgttaaactcctgatctaccatacatcaagattacactcaggagtcccatcaaggttcagtggcagtgggtctggaacagattacaccctcaccattagcagcctgcaaccggaagatattgccacttacttctgccaacagggtaatacgcttccgtacacgttcggaggggggaccaagctggagatcacaggtggcggtggctcgggcggtggtgggtcgggtggcggcggatctcaggtgcagctgcaggagtcaggacctggcctggtgaaaccctcacagactctgtccgtgacatgcactgtctcaggggtctcattacccgactatggtgtaagctggattcgccagcctccaggtaagggtctggagtggctgggagtaatatggggtagtgaaaccacatactataattcagctctcaaatccagactgaccatctccaaggacaactccaagagccaagtttccttaaaattaagtagtgttactgctgctgacacagccgtctactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 20 - protein, 706 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 21 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctattattgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 22 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 23 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccgggcggcaccgtgaaactgctgatttatcataccagccgcctgcatagcggcgtgccgagccgctttagcggcagcggcagcggcaccgattttaccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 24 - protein, 706 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 25 - DNA, 2150 bp, Synthetic construct
  source 1..2150: mol_type = other DNA
gaattcgccgccaccatggccttaccagtgaccgccttgctcctgccgctggccttgctgctccacgccgccaggccggatattcagatgacccagagcccgagcagcctgagcgcgagcgtgggcgatcgcgtgaccattacctgccgcgcgagccaggatattagcaaatatctgaactggtatcagcagaaaccggatggcaccgtgaaactgctgatttatcataccagccgcctgcatagcggcgtgccgagccgctttagcggcagcggcagcggcaccgattataccctgaccattagcagcctgcagccggaagatattgcgacctatttttgccagcagggcaacaccctgccgtatacctttggcggcggcaccaaactggaaattaccggtggcggtggctcgggcggtggtgggtcgggtggcggcggatctcaggtgcagctgcaggaaagcggcccgggcctggtgaaaccgagccagaccctgagcgtgacctgcaccgtgagcggcgtgagcctgccggattatggcgtgagctggattcgccagccgccgcgcaaaggcctggaatggctgggcgtgatttggggcagcgaaaccacctattataacagcgcgctgaaaagccgcctgaccattagcaaagataacagcaaaagccaggtgagcctgaaaatgagcagcgtgaccgcggcggataccgcgatttattattgcgcgaaacattattattatggcggcagctatgcgatggattattggggccagggcaccagcgtgaccgtgagcagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 26 - protein, 706 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 27 - DNA, 2210 bp, Synthetic construct
  source 1..2210: mol_type = other DNA
ggatccgaattcgccgccaccatggccttaccagtgaccgccttgctcctgccgctggccttgctgctccacgccgccaggccggactacaaagacgatgacgataaaggcggtggtggctctggtggtggcggcagcgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaagcggccgcgtcgac

SEQ ID NO: 28 - protein, 724 aa, Synthetic construct
MALPVTALLLPLALLLHAARPDYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 29 - RNA, 20 nt, Synthetic construct
  source 1..20: mol_type = other RNA
ctcaccagattcccgaaggt

SEQ ID NO: 30 - RNA, 20 nt, Synthetic construct
  source 1..20: mol_type = other RNA
ccgccttgtcatcaaccgtc

SEQ ID NO: 31 - RNA, 20 nt, Synthetic construct
  source 1..20: mol_type = other RNA
tctgcgttcaggggtaagcg

SEQ ID NO: 32 - RNA, 20 nt, Synthetic construct
  source 1..20: mol_type = other RNA
gcgcttacccctgaacgcag

SEQ ID NO: 33 - RNA, 20 nt, Synthetic construct
  source 1..20: mol_type = other RNA
cgcagaggaccatgtccccg

SEQ ID NO: 34 - RNA, 20 nt, Synthetic construct
  source 1..20: mol_type = other RNA
cccctaccatgactttattc

SEQ ID NO: 35 - RNA, 20 nt, Synthetic construct
  source 1..20: mol_type = other RNA
attgcactcatcagagctac

SEQ ID NO: 36 - RNA, 20 nt, Synthetic construct
  source 1..20: mol_type = other RNA
agtcatggtaggggagcttg

SEQ ID NO: 37 - RNA, 20 nt, Synthetic construct
  source 1..20: mol_type = other RNA
tgctggcgatacgcgtccac

SEQ ID NO: 38 - RNA, 20 nt, Synthetic construct
  source 1..20: mol_type = other RNA
gtgagcaatcccccgggcga

SEQ ID NO: 39 - RNA, 20 nt, Synthetic construct
  source 1..20: mol_type = other RNA
aacgtgcggtgggatcgtgc

SEQ ID NO: 40 - DNA, 1122 bp, Synthetic construct
  source 1..1122: mol_type = other DNA
atggccttaccagtgaccgccttgctcctgccgctggccttgctgctccacgccgccaggccgttattcaaccaagaagttcaaattcccttgaccgaaagttactgtggcccatgtcctaaaaactggatatgttacaaaaataactgctaccaattttttgatgagagtaaaaactggtatgagagccaggcttcttgtatgtctcaaaatgccagccttctgaaagtatacagcaaagaggaccaggatttacttaaactggtgaagtcatatcattggatgggactagtacacattccaacaaatggatcttggcagtgggaagatggctccattctctcacccaacctactaacaataattgaaatgcagaagggagactgtgcactctatgcctcgagctttaaaggctatatagaaaactgttcaactccaaatacgtacatctgcatgcaaaggactgtgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 41 - protein, 374 aa, Synthetic construct
MALPVTALLLPLALLLHAARPLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 2181 bp, Synthetic construct
  source 1..2181: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 43 - protein, 501 aa, Synthetic construct
MALPVTALLLPLALLLHAARPDYKDDDDKGGGGSGGGGSQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 2181 bp, Synthetic construct
  source 1..2181: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttcaggtggaggcggttcaggcggaggtggctctggcggtggcggatcggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 45 - protein, 501 aa, Synthetic construct
MALPVTALLLPLALLLHAARPDYKDDDDKGGGGSGGGGSQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 46 - DNA, 2172 bp, Synthetic construct
  source 1..2172: mol_type = other DNA
atggccttaccagtgaccgccttgctcctgccgctggccttgctgctccacgccgccaggccggactacaaagacgatgacgataaaggcggtggtggctctggtggtggcggatcccaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 47 - protein, 498 aa, Synthetic construct
MALPVTALLLPLALLLHAARPDYKDDDDKGGGGSGGGGSQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 48 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 50 - protein, 481 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 52 - protein, 482 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - protein, 481 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - protein, 481 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - protein, 481 aa, Synthetic construct
MALPVTALLLPLALLLHAARPDIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - protein, 481 aa, Synthetic construct
MALPVTALLLPLALLLHAARPDIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - DNA, 612 bp, Synthetic construct
  source 1..612: mol_type = other DNA
atggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgc

SEQ ID NO: 61 - protein, 204 aa, Synthetic construct
MALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 62 - DNA, 2127 bp, Synthetic construct
  source 1..2127: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccgattactatatgcactgggtgcgacaggcccctggacaagggcttgagtggatgggaataatcaaccctaatagtggtggaacaaattacgcacagaagttccagggcagagtcaccatgaccagggacacgtccacgagcacagtctacatggagctgagcagcctgagatctgaggacacggccgtgtattactgtgctagagattttagtgtgtatgctgctgcagatggcatggatgtctggggccaagggacattggtcaccgtctcttcaggtggaggcggttcaggcggaggtggctctggcggtggcggatcggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgacgcatccaatttggccaccggggtcccatcaaggttcagtggcagtggatctgggacagatttcactctcaccatcagcagtctgcaacctgaagattttgcaacttactactgtcaacagagtgatagttcacctctcactttcggccaagggaccaaggtggagatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 63 - DNA, 2118 bp, Synthetic construct
  source 1..2118: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccagctacgacattaattgggtgcgacaggcccctggacaagggcttgagtggatgggagtgatcaaccctagtggtggaggtacaagctacgcacagaagttccagggcagagtcaccatgaccagggacacgtccacgagcacagtctacatggagctgagcagcctgagatctgaggacacggccgtgtattactgtgctagatattctggctatgattgggtgtttgactactggggccaagggaccctggtcaccgtctcctcaggtggaggcggttcaggcggaggtggctctggcggtggcggatcggacatccagatgacccagtctccatcctccctgtctgcatctgtaggagacagagtcaccatcacttgccgggcaagtcagggcattggcagctggttagcttggtatcagcagaaaccagggaaagcccctaagctcctgatctatgctgcatccacattgcaaagtggggtcccatcaaggttcagtggcagtggatctgggacagatttcactctcaccatcagcagtctgcaacctgaagattttgcaacttactactgtcaacagagttacagtaccccttggactttcggccctgggaccaaagtggatatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 64 - DNA, 2127 bp, Synthetic construct
  source 1..2127: mol_type = other DNA
atggccttaccagtgaccgccttgctcctgccgctggccttgctgctccacgccgccaggccgcaggtgcagctggtgcagtctggggctgaggtgaagaagcctggggcctcagtgaaggtttcctgcaaggcatctggatacaccttcaccagctacgacatcaattgggtgcgacaggcccctggacaagggcttgagtggatgggagtcatcagccctagtggtggtggaacaagctacgcacagaagttccagggcagagtcaccatgaccagggacacgtccacgagcacagtctacatggagctgagcagcctgagatctgaggacacggccgtgtattactgtgctagatactacggcggcaaccccgaagggtggtacttcgatctctggggccgtggcaccctggtcactgtctcctcaggtggaggcggttcaggcggaggtggctctggcggtggcggatcggacatccagatgacccagtctccatcctccctgtctgcatctgtaggagacagagtcaccatcacttgccgggcaagtcagagcattggaaattggttagcctggtatcagcagaaaccagggaaagcccctaagctcctgatctatgacgcatccgatttggaaaccggggtcccatcaaggttcagtggcagtggatctgggacagatttcactctcaccatcagcagtctgcaacctgaagattttgcaacttactactgtcaacagagttacactaccccttggacgttcggccaagggaccaaggtggaaatcaaaaccacgacgccagcgccgcggccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccggagggaccagaggctgccccccgatgcccacaagccccctgggggaggcagtttccggacccccatccaagaggagcaggccgacgcccactccaccctggccaagatcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctctggcgagggaaggggttccctgcttacttgcggcgacgtcgaagagaatcccggtccgatggccctcccagtaactgccctccttttgcccctcgcactccttcttcatgccgctcgccccaactgggtcaacgtgattagcgatttgaagaaaatcgaggaccttatacagtctatgcatattgacgctacactgtatactgagagtgatgtacacccgtcctgtaaggtaacggccatgaaatgctttcttctggagctccaggtcatcagcttggagtctggggacgcaagcatccacgatacggttgaaaacctcatcatccttgcgaacaactctctctcatctaatggaaacgttacagagagtgggtgtaaggagtgcgaagagttggaagaaaaaaacatcaaagaatttcttcaatccttcgttcacatagtgcaaatgttcattaacacgtccactaccacacccgccccgaggccacctacgccggcaccgactatcgccagtcaacccctctctctgcgccccgaggcttgccggcctgcggctggtggggcggtccacacccggggcctggattttgcgtgcgatatatacatctgggcacctcttgccggcacctgcggagtgctgcttctctcactcgttattacgctgtactgctaa

SEQ ID NO: 65 - protein, 708 aa, Synthetic construct
MALPVTALLLPLALLLHAARPQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 66 - protein, 705 aa, Synthetic construct
MALPVTALLLPLALLLHAARPQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 67 - protein, 708 aa, Synthetic construct
MALPVTALLLPLALLLHAARPQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 68 - protein, 483 aa, Synthetic construct
MALPVTALLLPLALLLHAARPQVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - protein, 480 aa, Synthetic construct
MALPVTALLLPLALLLHAARPQVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 483 aa, Synthetic construct
MALPVTALLLPLALLLHAARPQVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 578 aa, Synthetic construct
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 72 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 73 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 74 - protein, 685 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 75 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 76 - protein, 686 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 77 - protein, 685 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 78 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 79 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 80 - protein, 685 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 81 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 82 - protein, 685 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 83 - protein, 685 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 84 - protein, 703 aa, Synthetic construct
DYKDDDDKGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 85 - protein, 353 aa, Synthetic construct
LFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTVTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 460 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWIGVIWGSETTYYNSALKSRLTISKDNSKNQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 461 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 460 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSLTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 94 - protein, 460 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPGKGLEWLGVIWGSETTYYNSALKSRLTISKDNSKSQVSLKLSSVTAADTAVYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYYCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 460 aa, Synthetic construct
DIQMTQSPSSLSASVGDRVTITCRASQDISKYLNWYQQKPGGTVKLLIYHTSRLHSGVPSRFSGSGSGTDFTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 97 - protein, 460 aa, Synthetic construct
DIQMTQSPSSLSASVGDRVTITCRASQDISKYLNWYQQKPDGTVKLLIYHTSRLHSGVPSRFSGSGSGTDYTLTISSLQPEDIATYFCQQGNTLPYTFGGGTKLEITGGGGSGGGGSGGGGSQVQLQESGPGLVKPSQTLSVTCTVSGVSLPDYGVSWIRQPPRKGLEWLGVIWGSETTYYNSALKSRLTISKDNSKSQVSLKMSSVTAADTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 183 aa, Synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 99 - protein, 687 aa, Synthetic construct
QVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 100 - protein, 684 aa, Synthetic construct
QVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 101 - protein, 687 aa, Synthetic construct
QVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSTTTPAPRPPTPAPTIASQPLSLRPEACRPAAGGAVHTRGLDFACDIYIWAPLAGTCGVLLLSLVITLYC

SEQ ID NO: 102 - protein, 462 aa, Synthetic construct
QVQLVQSGAEVKKPGASVKVSCKASGYTFTDYYMHWVRQAPGQGLEWMGIINPNSGGTNYAQKFQGRVTMTRDTSTSTVYMELSSLRSEDTAVYYCARDFSVYAAADGMDVWGQGTLVTVSSGGGGSGGGGSGGGGSDIQMTQSPSSLSASVGDRVTITCRASQSISSYLNWYQQKPGKAPKLLIYDASNLATGVPSRFSGSGSGTDFTLTISSLQPEDFATYYCQQSDSSPLTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 103 - protein, 459 aa, Synthetic construct
QVQLVQSGAEVKKPGASVKVSCKASGYTFTSYDINWVRQAPGQGLEWMGVINPSGGGTSYAQKFQGRVTMTRDTSTSTVYMELSSLRSEDTAVYYCARYSGYDWVFDYWGQGTLVTVSSGGGGSGGGGSGGGGSDIQMTQSPSSLSASVGDRVTITCRASQGIGSWLAWYQQKPGKAPKLLIYAASTLQSGVPSRFSGSGSGTDFTLTISSLQPEDFATYYCQQSYSTPWTFGPGTKVDIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 104 - protein, 462 aa, Synthetic construct
QVQLVQSGAEVKKPGASVKVSCKASGYTFTSYDINWVRQAPGQGLEWMGVISPSGGGTSYAQKFQGRVTMTRDTSTSTVYMELSSLRSEDTAVYYCARYYGGNPEGWYFDLWGRGTLVTVSSGGGGSGGGGSGGGGSDIQMTQSPSSLSASVGDRVTITCRASQSIGNWLAWYQQKPGKAPKLLIYDASDLETGVPSRFSGSGSGTDFTLTISSLQPEDFATYYCQQSYTTPWTFGQGTKVEIKTTTPAPRPPTPAPTIASQPLSLRPEACRPAAGGAVHTRGLDFACDIYIWAPLAGTCGVLLLSLVITLYCRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - DNA, 14 bp, Synthetic construct
  source 1..14: mol_type = other DNA
atgctcaatgcgtc

SEQ ID NO: 106 - RNA, 14 nt, Synthetic construct
  source 1..14: mol_type = other RNA
atgctcaatgcgtc

SEQ ID NO: 107 - DNA, 14 bp, Synthetic construct
  source 1..14: mol_type = other DNA
tacgagttacgcag